(19) United States
(12) Patent Application Publication
(10) Pub. No.: US 2023/0416261 A1
(43) Pub. Date: Dec. 28, 2023

(54) ETHANEDIAMINE-HETEROCYCLE DERIVATIVES AS INHIBITORS OF PROTEIN ARGININE METHYLTRANSFERASES

(71) Applicant: Board of Regents, The University of Texas System, Austin, TX (US)

(72) Inventors: Maria Emilia DI FRANCESCO, Houston, TX (US); Philip JONES, Houston, TX (US); Timothy MCAFOOS, Pearland, TX (US); Christopher L. CARROLL, Houston, TX (US); Zhijun KANG, Richmond, TX (US)

(21) Appl. No.: 18/461,735

(22) Filed: Sep. 6, 2023

Related U.S. Application Data
(63) Continuation of application No. PCT/US2022/019729, filed on Mar. 10, 2022.
(60) Provisional application No. 63/280,234, filed on Nov. 17, 2021.
(60) Provisional application No. 63/160,189, filed on Mar. 12, 2021.

(51) Int. Cl. C07D 487/04; A61P 35/00; A61K 45/06

(52) U.S. Cl. CPC C07D 487/04; A61K 45/06; A61P 35/00

(57) ABSTRACT

Provided are ethanediamine-heterocycle compounds, and salts thereof, that are able to act as inhibitors of PRMTs (protein arginine methyltransferases) for treating cancer and other diseases mediated by PRMTs.

This application is a bypass continuation of International Application No. PCT/US2022/019729, filed Mar. 10, 2022, which claims the benefit of priority of U.S. Provisional Application No. 63/160,189 filed Mar. 12, 2021, and U.S. Provisional Application No. 63/280,234 filed Nov. 17, 2021, the disclosures of each are hereby incorporated by reference as if written herein in their entireties.

Disclosed herein are new heterocyclic compounds, and salts and compositions thereof, and their application as pharmaceuticals for the treatment of disease. Methods of inhibition of protein arginine methyltransferases (PRMTs) activity, in particular type I PRMTs, in a human or animal subject are also provided for the treatment diseases such as cancer.

Eukaryotic cells contain enzymes that are capable of posttranslational modification of amino acid residues in proteins. This modification has been shown to affect the function, localization, and stability of proteins. Introduction of methyl (—CH₃) groups in amino acid moieties, a process known as methylation, is a common means employed by organisms for altering the behavior of proteins, particularly those containing arginine residues. Methylation of arginine residues is perhaps the most widespread methylation pathways in mammalian cells. Arginine (“Arg” or “R”) contains a side-chain guanidine moiety, having the chemical formula —N═C(NH₂)₂, or a tautomer thereof. Methylation of arginine residues can be expected to modulate significantly the function of a protein, due to disruption of both ion pairs and hydrogen bonds, and introduction of unfavorable steric interactions.

An expanding class of enzymes termed “protein arginine N-methyltransferases” (“PRMTs”) performs both mono- and dimethylation of the guanidine functionality of arginine residues in proteins. These enzymes incorporate methyltransferase functionality that utilizes S-adenosyl methionine as a methyl group donor. PRMTs comprises two major types, termed I and II. Both type I and type II enzymes catalyze monomethylating of the guanidine of arginine, forming —N═C(NH₂)(NHCH₃) moiety (and tautomers). Type I enzymes also promote asymmetrical dimethylation of arginine residues, forming the —N═C(NH₂)(N(CH₃)₂) moieties (and tautomers). In contrast, type II enzymes promote symmetrical dimethylation of arginine residues, forming the —N═C(NHCH₃)₂ moiety (and tautomers). Also included in the PRMT class of enzymes are type III enzymes, which produces only the monomethylated product, and type IV enzymes, which methylate the non-terminal guanidine nitrogen of arginine residues, forming the —N(CH₃)—C(═NH)(NH₂) moiety (and tautomers).

Nine PRMTs have been identified in humans; each has a methyltransferase domain. Of these, PRMT1, a type I PRMT, is the primary enzyme responsible for the asymmetric dimethylation of arginine residues in yeast, trypanosomes, and humans. The protein is found in both the cytoplasm and the nucleus of yeast and human cells, and performs asymmetric dimethylation on both histones as well as a large number of non-histone protein substrates. Arginine methylation has been involved in several key cellular processes, including signal transduction, regulation of gene transcription, mRNA splicing and DNA repair, and overexpression of PRMTs has often been associated with various cancers. For example, overexpression of PRMT1 has been observed in numerous cancers, including breast, prostate, lung, colon, bladder cancers and leukemia. Therefore, inhibition of PRMTs, including inhibition of type I PRMTs, can be effective in the treatment of cancer. Similarly, PRMTs have been associated with additional diseases including diabetes, cardiovascular, renal and muscular diseases. Inhibition of PRMTs, including inhibition of type I PRMTs, can be effective in the treatment of such diseases.

Novel compounds, salts thereof, and pharmaceutical compositions, certain of which have been found to inhibit type I PRMTs, in particular PRMT1 have been discovered, together with methods of synthesizing and using the compounds and salts thereof including methods for the treatment of type I PRMT-mediated diseases in a patient by administering the compounds, and salts thereof.

### DETAILED DESCRIPTION

Provided herein is a compound of structural Formula I:

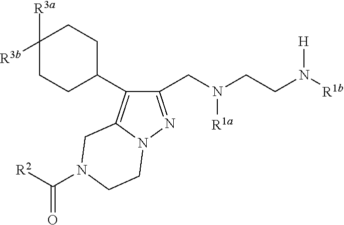

or a salt thereof, wherein:
R^{1a} and R^{1b} are independently chosen from H and CH₃;
R² is chosen from C₁₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.
R^{3a} and R^{3b} are independently chosen from (C₁₋₆alkoxy)C₁₋₆alkyl and (haloC₁₋₆alkoxy)C₁₋₆alkyl;
each R⁴ is independently chosen from halo, cyano, hydroxy, C₁₋₆haloalkyl, C₁₋₆alkoxy, and C₁₋₆haloalkoxy, and if R² is C₃₋₇cycloalkyl, 4- to 7-membered cycloalkoxy, or (C₃₋₇cycloalkyl)C₁₋₆alkyl, then R⁴ also may be C₁₋₆alkyl; and
provided the compound is not

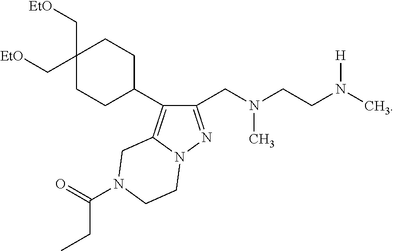 [SMILES: CCOCC1(COCC)CCC(c2c(CN(C)CCNC)nn3c2CN(C(=O)CC)CC3)CC1]

Certain compounds disclosed herein, or salts thereof, may possess useful type I PRMTs inhibiting activity, and may be used in the treatment or prophylaxis of a disease or condition in which type I PRMTs play an active role. Thus, in broad aspect, certain embodiments also provide pharmaceutical compositions comprising one or more compounds disclosed herein, or a salt or salts thereof, together with a pharmaceutically acceptable carrier, as well as methods of making and using the compounds, or salts thereof, and compositions. Certain embodiments provide methods for inhibiting type I PRMTs. Other embodiments provide methods for treating a PRMT-mediated disorder in a patient in need of such treatment, comprising administering to said patient a therapeutically effective amount of a compound disclosed herein, or salt or composition thereof. Also provided is the use of certain compounds disclosed herein, or salts thereof, for use in the manufacture of a medicament for the treatment of a disease or condition ameliorated by the inhibition of type I PRMTs.

In certain embodiments, R^{1b} is H. In certain embodiments, R^{1b} is CH₃.

In certain embodiments:
R² is chosen from methyl, C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴, or
R² is ethyl and is substituted with one or more R⁴.

In certain embodiments, R² is chosen from methyl, C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.

In certain embodiments:
R² is chosen from C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴, or
R² is C₁₋₂alkyl and is substituted with one or more R⁴.

In certain embodiments, R² is chosen from C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.

In certain embodiments:
R² is chosen from C₄₋₆alkyl, C₃₋₇cycloalkyl, 4- to 7-membered cycloalkoxy, and (C₃₋₇cycloalkyl)C₁₋₆alkyl, any of which is optionally substituted with one or more R⁴, or
R² is C₁₋₃alkyl and is substituted with one or more R⁴.

In certain embodiments, R² is chosen from C₄₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.

In certain embodiments, R² is chosen from C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.

In certain embodiments, R² is chosen from C₃₋₅alkyl and C₄₋₆cycloalkyl, either of which is optionally substituted with one or more R⁴.

In certain embodiments, R² is chosen from C₃₋₅alkyl, C₃₋₅cycloalkyl, and 4- to 6-membered cycloalkoxy, any of which is optionally substituted with one or more R⁴. In certain embodiments, R² is chosen from C₄₋₅alkyl, C₄₋₅cycloalkyl, and 4- to 6-membered cycloalkoxy, any of which is optionally substituted with one or more R⁴. In certain embodiments, R² is chosen from C₄alkyl, cyclobutyl, and oxetan-3-yl, any of which is optionally substituted with one or more R⁴.

In certain embodiments, R² is chosen from C₃₋₅alkyl and C₃₋₅cycloalkyl, either of which is optionally substituted with one or more R⁴. In certain embodiments, R² is chosen from C₄₋₅alkyl and C₄₋₅cycloalkyl, either of which is optionally substituted with one or more R⁴. In certain embodiments, R² is chosen from C₄alkyl and C₄cycloalkyl, either of which is optionally substituted with one or more R⁴.

In certain embodiments, R² is not ethyl. In certain embodiments, R² is neither methyl nor ethyl.

In certain embodiments, R² is optionally substituted with one or two R⁴. In certain embodiments, R² is optionally substituted with one R⁴. In certain embodiments, R² is substituted with one or more R⁴. In certain embodiments, R² is not substituted with an R⁴.

In certain embodiments, R² is chosen from cylopropyl, cyclobutyl, cyclopentyl, 2-propyl, 2-butyl, and 2-methyl-2-propyl. In certain embodiments, R² is chosen from cyclobutyl and 2-methyl-2-propyl.

In certain embodiments, R² is chosen from:

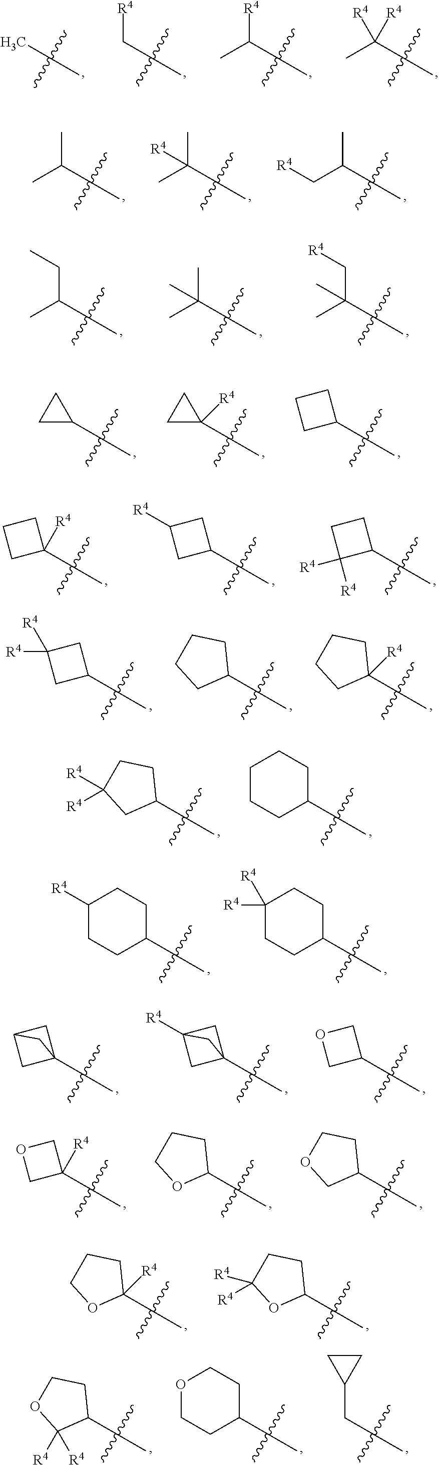

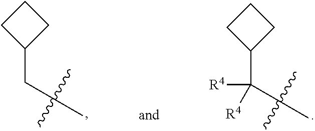

In certain embodiments:
R² is chosen from

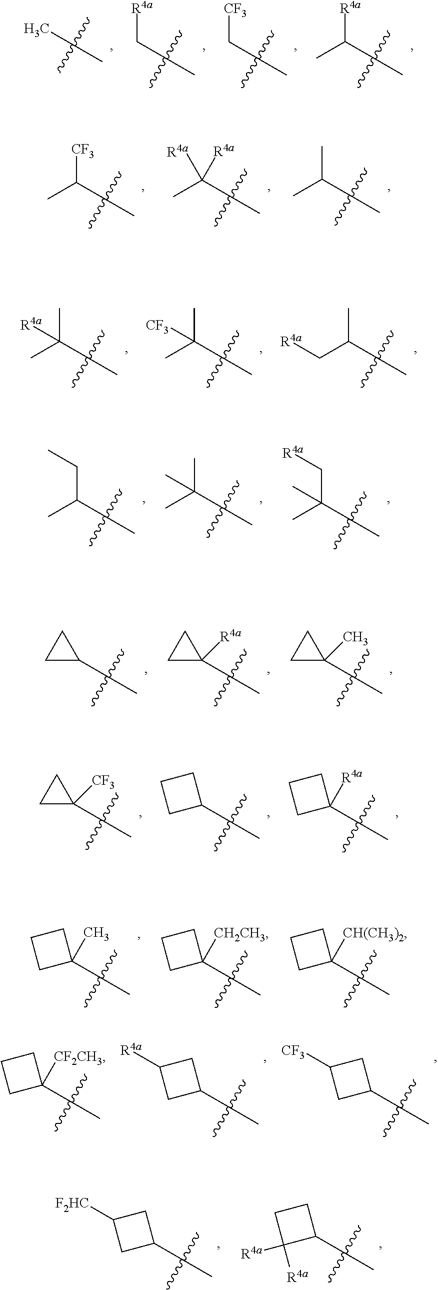

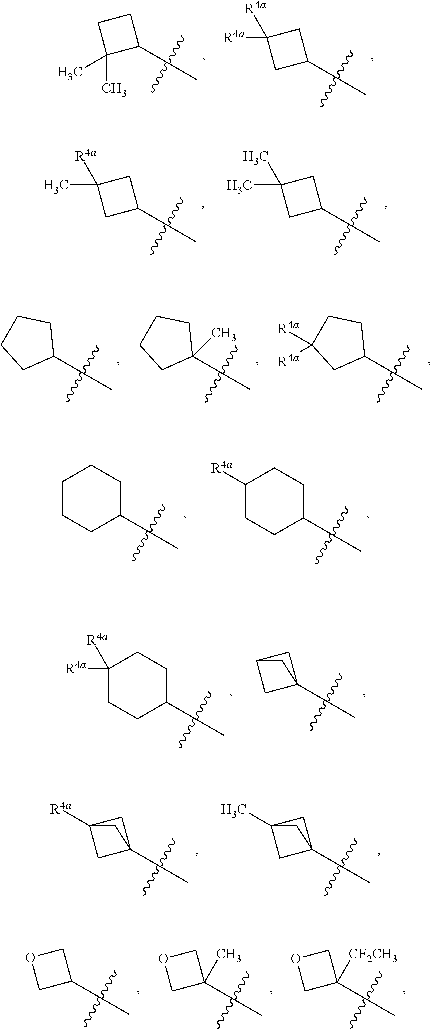

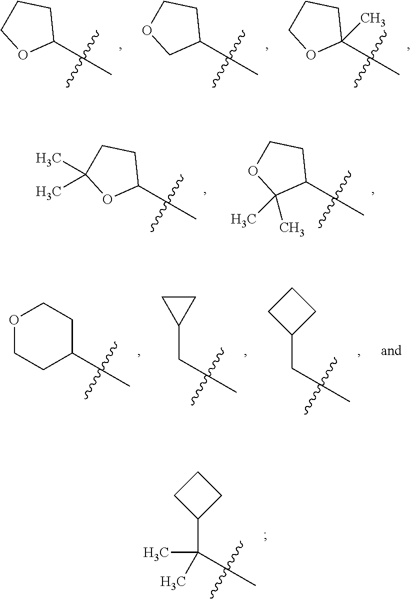

and
each R^{4a} is independently chosen from halo, cyano, hydroxy, C₁₋₄fluoroalkyl, C₁₋₄alkoxy, and C₁₋₄fluoroalkoxy.

In certain embodiments, each R^{4a} is independently chosen from halo, cyano, hydroxy, C₁₋₄alkoxy, and C₁₋₄fluoroalkoxy.

In certain embodiments:
R² b is chosen from

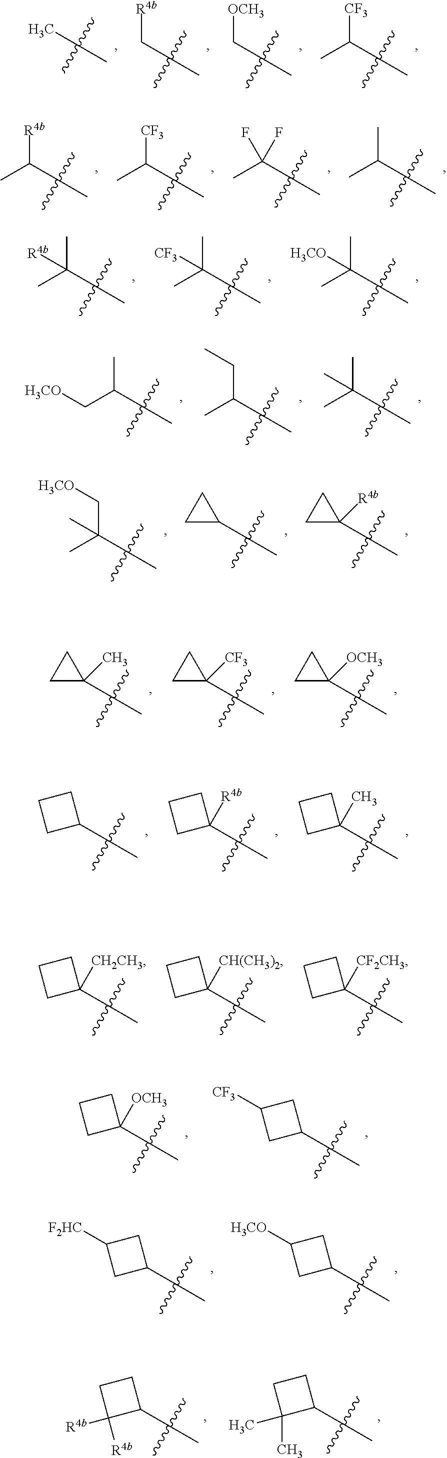

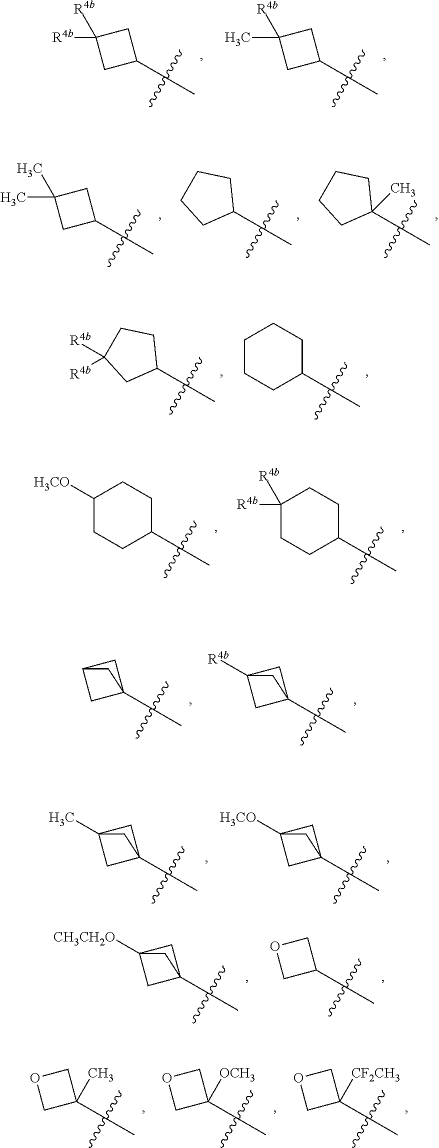

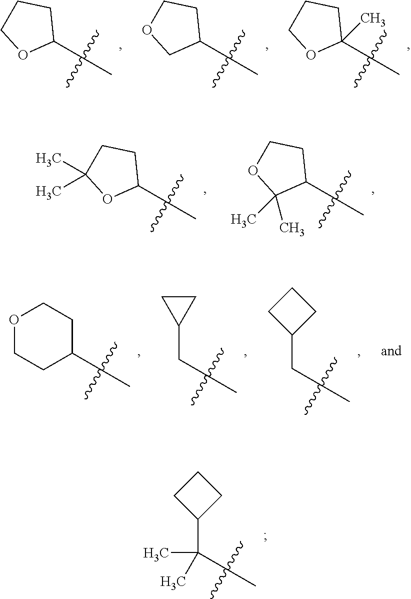

and
each R^{4b} is independently chosen from halo, cyano, hydroxy, C₁₋₄fluoroalkyl, C₁₋₄alkoxy, and C₁₋₄fluoroalkoxy.

In certain embodiments, each R^{4b} is independently chosen from halo, cyano, hydroxy, C₁₋₄alkoxy, and C₁₋₄fluoroalkoxy. In certain embodiments, each R^{4b} is independently chosen from halo, cyano, and hydroxy. In certain embodiments, each R^{4b} is independently chosen from fluoro, chloro, cyano, and hydroxy.

In certain embodiments:
R² is chosen from

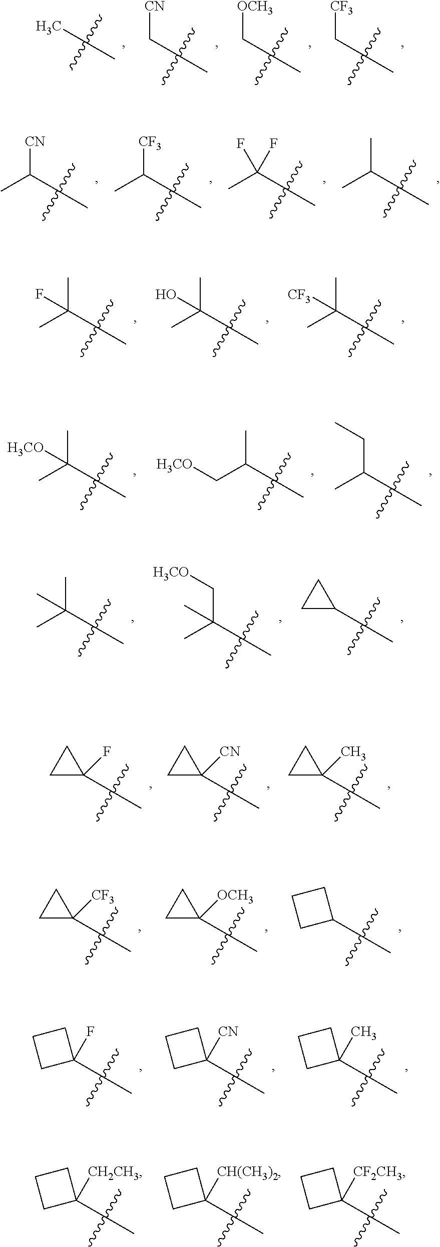

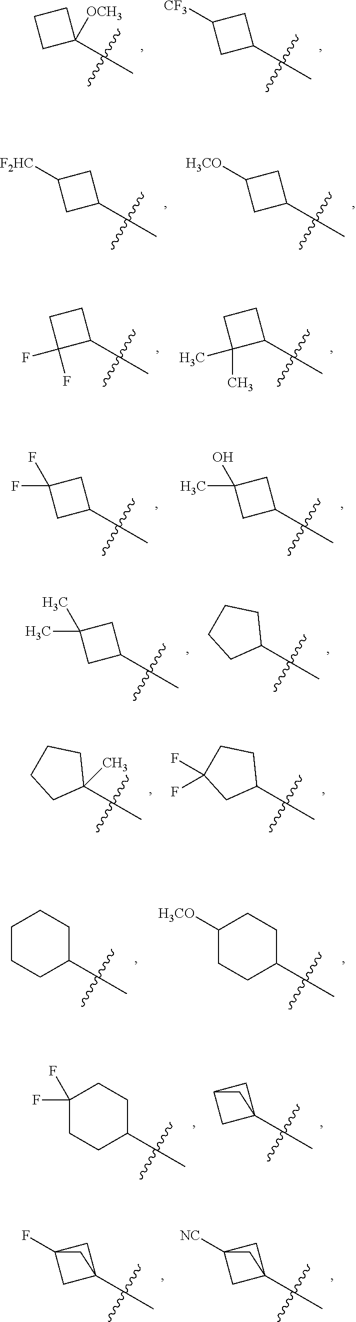

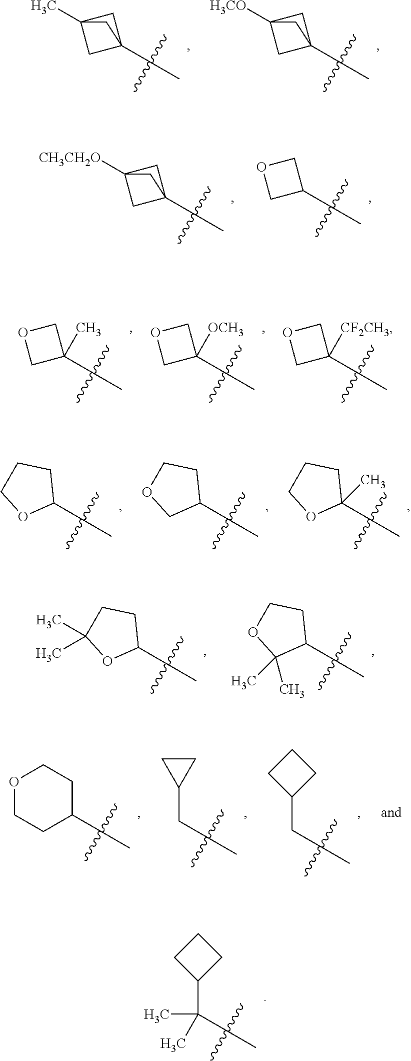

In certain embodiments, R² is chosen from CH₃, CH(CH₃)₂, CH(CH₃)CH₂CH₃, C(CH₃)₃, CH₂CN, CH₂OCH₃, CH₂CF₃, CH(CN)CH₃, CH(CF₃)CH₃, CF₂CH₃, CF(CH₃)₂, C(CN)(CH₃)₂, C(OH)(CH₃)₂, C(OCH₃)(CH₃)₂, CH(CH₃)(CH₂OCH₃), C(CH₃)₂(CH₂OCH₃), C(CH₃)₂(CF₃), cyclopropyl, cyclobutyl, cyclopentyl, cyclohexyl, bicyclo[1.1.1]pentan-1-yl, oxetan-3-yl, tetrahydrofuran-2-yl, tetrahydrofuran-3-yl, tetrahydro-2H-pyran-4-yl, 1-fluorocyclopropyl, 1-cyanocyclopropyl, 1-methylcyclopropyl, 1-(trifluoromethyl)cyclopropyl, 1-methoxycyclopropyl, 1-cyanocyclobutyl, 1-methylcyclobutyl, 1-ethylcyclobutyl, 1-(2-propyl)cyclobutyl, 1-methoxycyclobutyl, 3-methoxycyclobutyl, 3-(trifluoromethyl)cyclobutyl, 2,2-difluorocyclobutyl, 3,3-difluorocyclobutyl, 3,3-dimethylcyclobutyl, 3-hydroxy-3-methylcyclobutyl, 1-methylcyclopentyl, 3,3-difluorocyclopentyl, 4-methoxycyclohexyl, 4,4-difluorocyclohexyl, 3-fluorobicyclo[1.1.1]pentan-1-yl, 3-cyanobicyclo[1.1.1]pentan-1-yl, 3-methylbicyclo[1.1.1]pentan-1-yl, 3-methoxybicyclo[1.1.1]pentan-1-yl, 3-ethoxybicyclo[1.1.1]pentan-1-yl, 3-methyloxetan-3-yl, 3-(1,1-difluoroethyl)oxetan-3-yl, 2-methyltetrahydrofuran-2-yl, 5,5-dimethyltetrahydrofuran-2-yl, 3-methyltetrahydrofuran-3-yl, 2,2-dimethyltetrahydrofuran-3-yl, (cyclopropyl)methyl, and (cyclobutyl)methyl.

In certain embodiments, R^{3a} and R^{3b} are independently chosen from (C₁₋₆alkoxy)-methyl and (haloC₁₋₆alkoxy)methyl. In certain embodiments, R^{3a} and R^{3b} are independently chosen from (C₁₋₄alkoxy)methyl and (haloC₁₋₄alkoxy)methyl. In certain embodiments, R^{3a} and R^{3b} are independently chosen from (methoxy)methyl and (ethoxy)methyl. In certain embodiments, R^{3a} and R^{3b} are (methoxy)methyl. In certain embodiments, R^{3a} and R^{3b} are identical.

In certain embodiments, each R⁴ is independently chosen from halo, cyano, hydroxy, C₁₋₄fluoroalkyl, C₁₋₄alkoxy, and C₁₋₄fluoroalkoxy, and if R² is C₃₋₇cycloalkyl, 4- to 7-membered cycloalkoxy, or (C₃₋₇cycloalkyl)C₁₋₆alkyl, then R⁴ also may be C₁₋₄alkyl.

In certain embodiments, each R⁴ is independently chosen from halo, cyano, hydroxy, C₁₋₄fluoroalkyl, C₁₋₄alkoxy, and C₁₋₄fluoroalkoxy.

In certain embodiments, each R⁴ is independently chosen from halo, cyano, hydroxy, C₁₋₄alkoxy, and C₁₋₄fluoroalkoxy.

In certain embodiments, each R⁴ is independently chosen from halo, cyano, and hydroxy.

In certain embodiments, each R⁴ is independently chosen from fluoro, chloro, cyano, and hydroxy.

In certain embodiments, R⁴ is fluoro.

In certain embodiments, each R⁴ is the same.

Also provided is a compound chosen from:

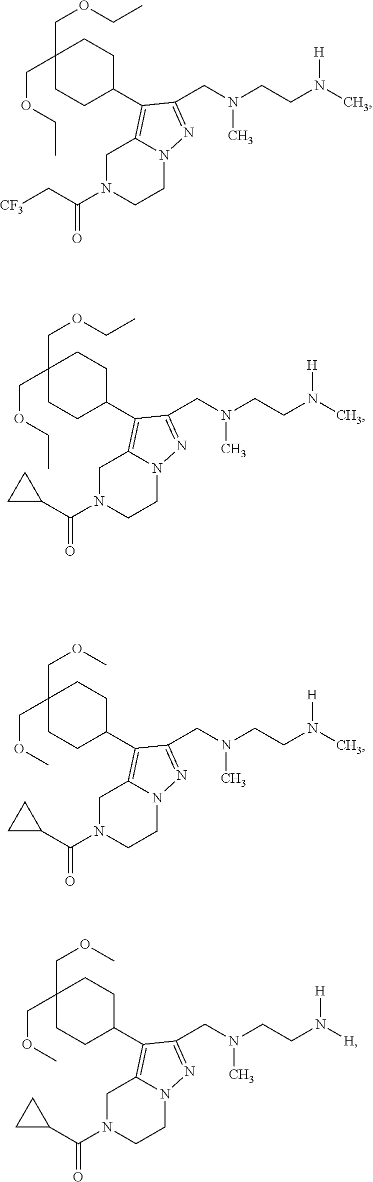

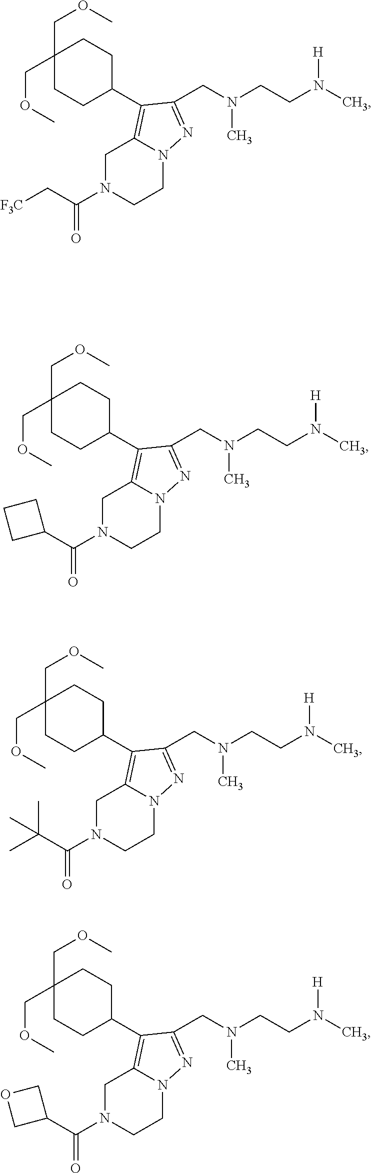 [SMILES: CNCCN(C)Cc1nn2c(c1C1CCC(COC)(COC)CC1)CN(C(=O)CC(F)(F)F)CC2 ; CNCCN(C)Cc1nn2c(c1C1CCC(COC)(COC)CC1)CN(C(=O)C1CCC1)CC2 ; CNCCN(C)Cc1nn2c(c1C1CCC(COC)(COC)CC1)CN(C(=O)C(C)(C)C)CC2 ; CNCCN(C)Cc1nn2c(c1C1CCC(COC)(COC)CC1)CN(C(=O)C1COC1)CC2]

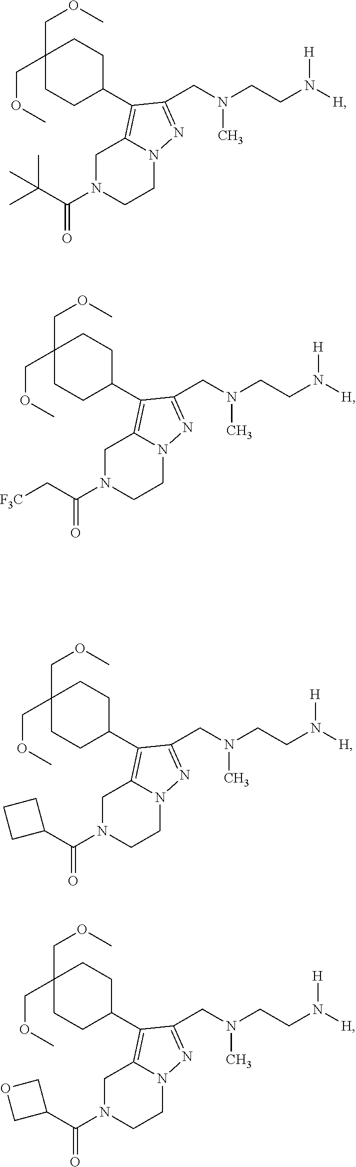

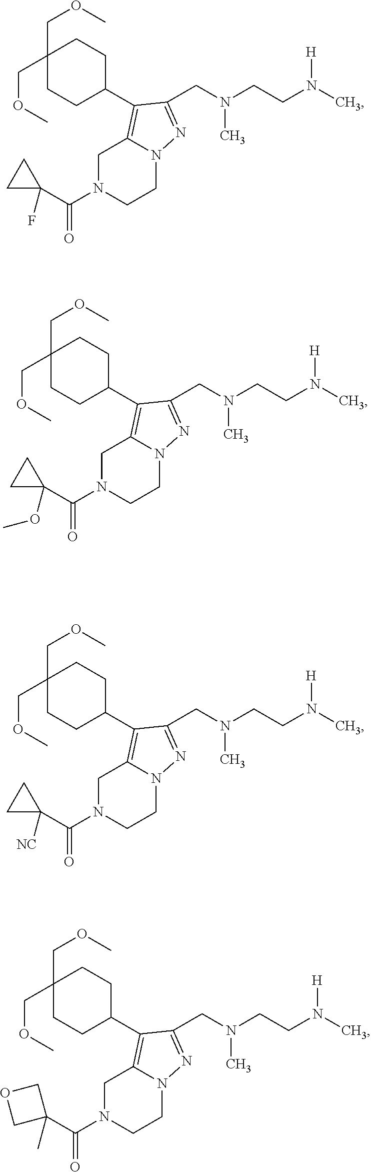 [SMILES: CNCCN(C)Cc1nn2c(c1C1CCC(COC)(COC)CC1)CN(C(=O)C1(F)CC1)CC2 ; CNCCN(C)Cc1nn2c(c1C1CCC(COC)(COC)CC1)CN(C(=O)C1(OC)CC1)CC2 ; CNCCN(C)Cc1nn2c(c1C1CCC(COC)(COC)CC1)CN(C(=O)C1(C#N)CC1)CC2 ; CNCCN(C)Cc1nn2c(c1C1CCC(COC)(COC)CC1)CN(C(=O)C1(C)COC1)CC2]

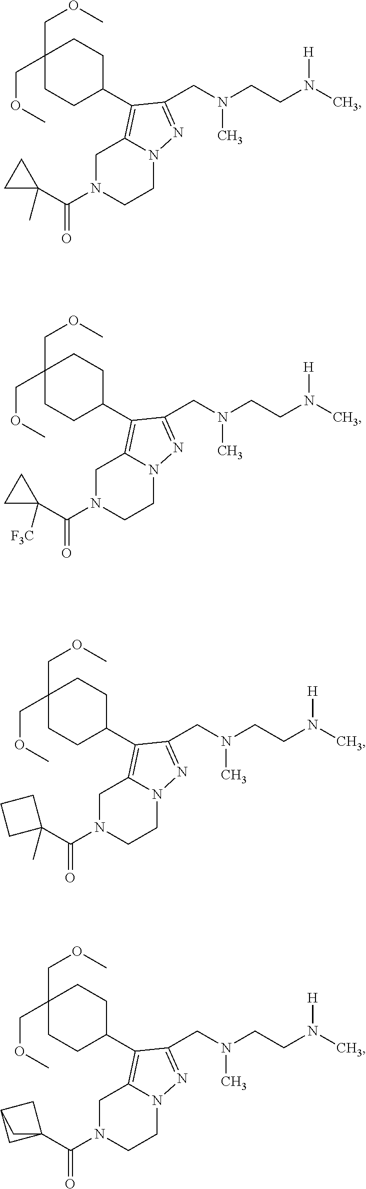 [SMILES: CNCCN(C)Cc1nn2c(c1C1CCC(COC)(COC)CC1)CN(C(=O)C1(C)CC1)CC2 ; CNCCN(C)Cc1nn2c(c1C1CCC(COC)(COC)CC1)CN(C(=O)C1(C(F)(F)F)CC1)CC2 ; CNCCN(C)Cc1nn2c(c1C1CCC(COC)(COC)CC1)CN(C(=O)C1(C)CCC1)CC2 ; CNCCN(C)Cc1nn2c(c1C1CCC(COC)(COC)CC1)CN(C(=O)C13CC(C1)C3)CC2]

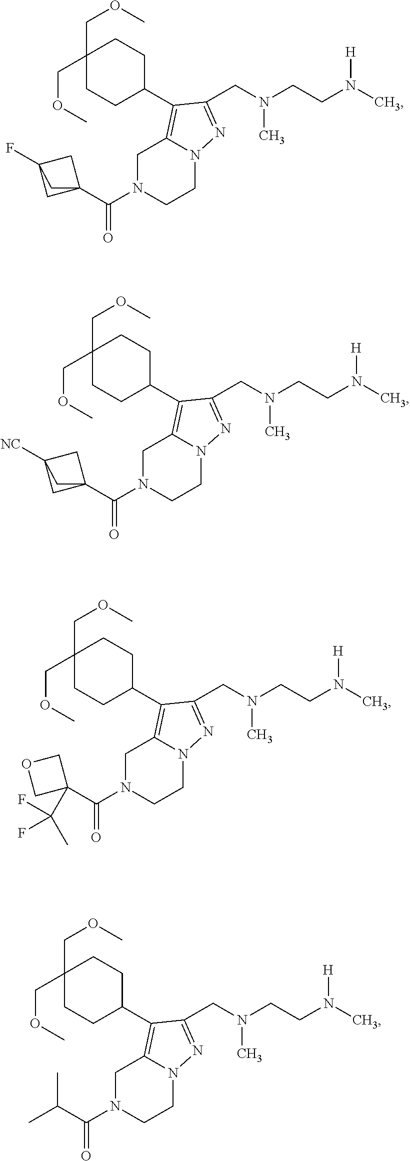 [SMILES: CNCCN(C)Cc1nn2c(c1C1CCC(COC)(COC)CC1)CN(C(=O)C13CC(F)(C1)C3)CC2 ; CNCCN(C)Cc1nn2c(c1C1CCC(COC)(COC)CC1)CN(C(=O)C13CC(C#N)(C1)C3)CC2 ; CNCCN(C)Cc1nn2c(c1C1CCC(COC)(COC)CC1)CN(C(=O)C1(C(C)(F)F)COC1)CC2 ; CNCCN(C)Cc1nn2c(c1C1CCC(COC)(COC)CC1)CN(C(=O)C(C)C)CC2]

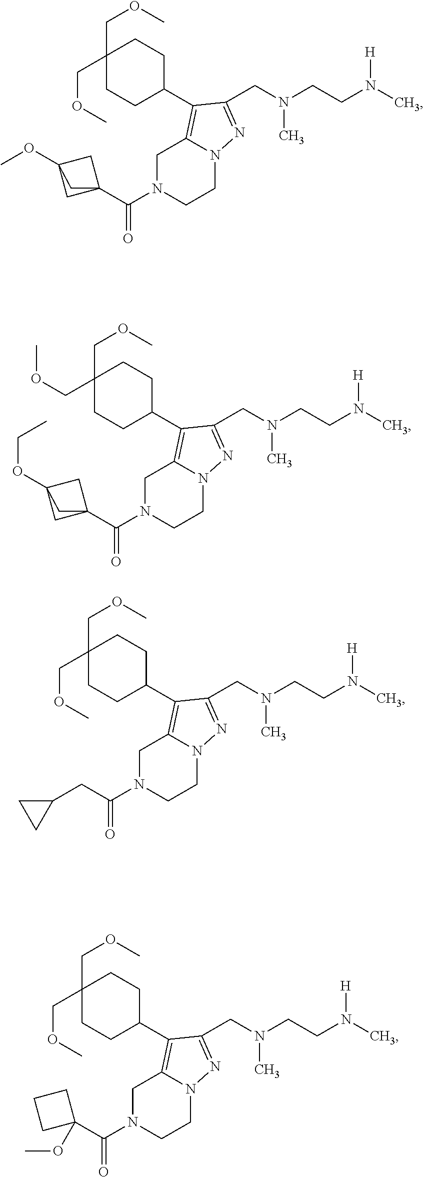

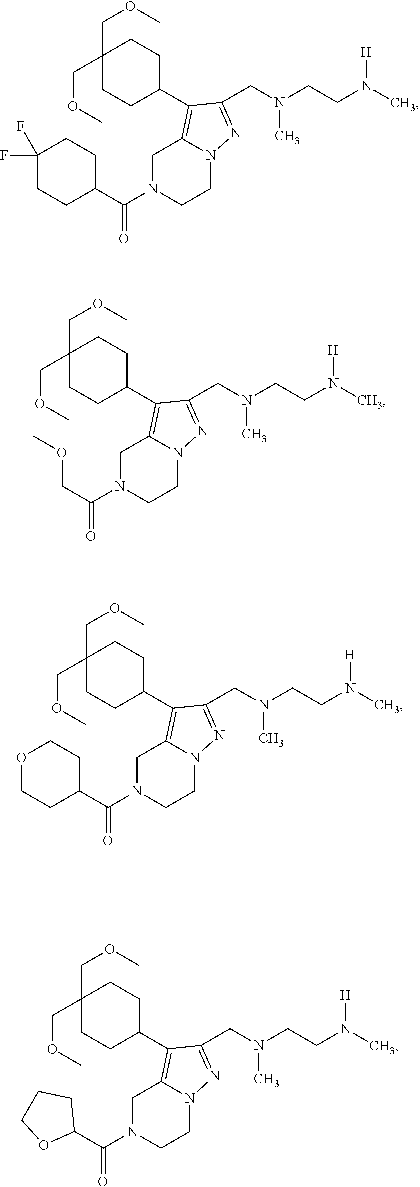 [SMILES: CNCCN(C)Cc1nn2c(c1C1CCC(COC)(COC)CC1)CN(C(=O)C1CCC(F)(F)CC1)CC2 ; CNCCN(C)Cc1nn2c(c1C1CCC(COC)(COC)CC1)CN(C(=O)COC)CC2 ; CNCCN(C)Cc1nn2c(c1C1CCC(COC)(COC)CC1)CN(C(=O)C1CCOCC1)CC2 ; CNCCN(C)Cc1nn2c(c1C1CCC(COC)(COC)CC1)CN(C(=O)C1CCCO1)CC2]

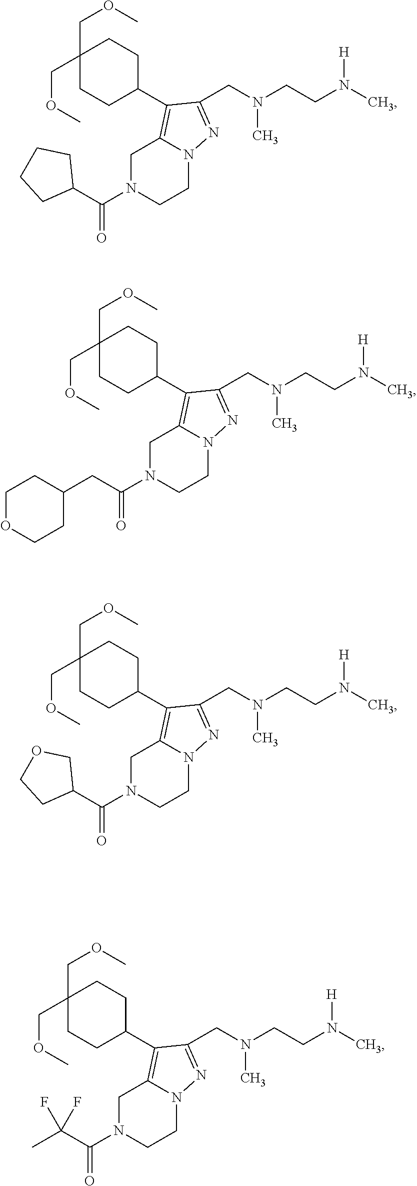 [SMILES: CNCCN(C)Cc1nn2c(c1C1CCC(COC)(COC)CC1)CN(C(=O)C1CCCC1)CC2 ; CNCCN(C)Cc1nn2c(c1C1CCC(COC)(COC)CC1)CN(C(=O)CC1CCOCC1)CC2 ; CNCCN(C)Cc1nn2c(c1C1CCC(COC)(COC)CC1)CN(C(=O)C1CCOC1)CC2 ; CNCCN(C)Cc1nn2c(c1C1CCC(COC)(COC)CC1)CN(C(=O)C(C)(F)F)CC2]

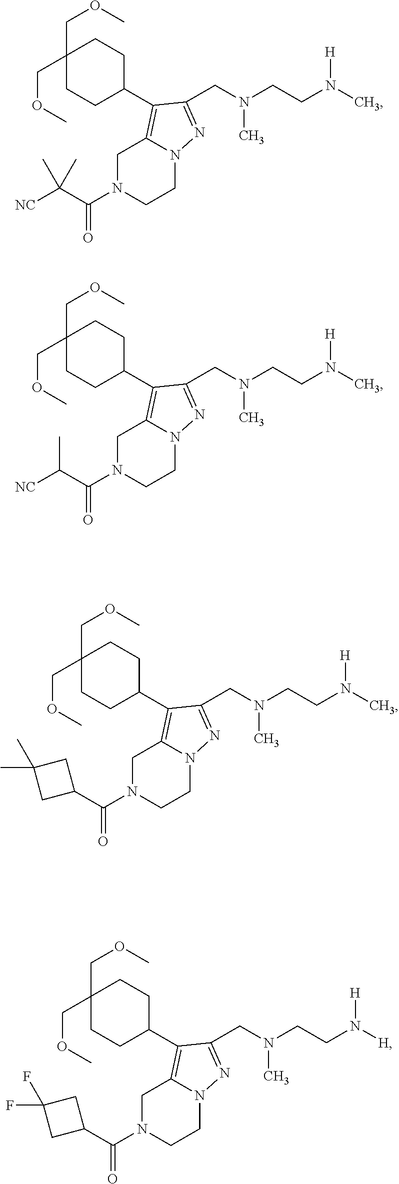

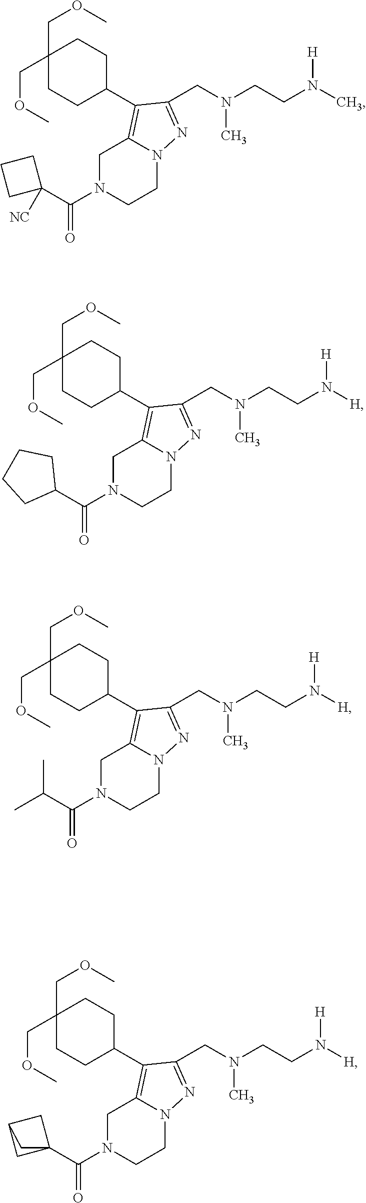

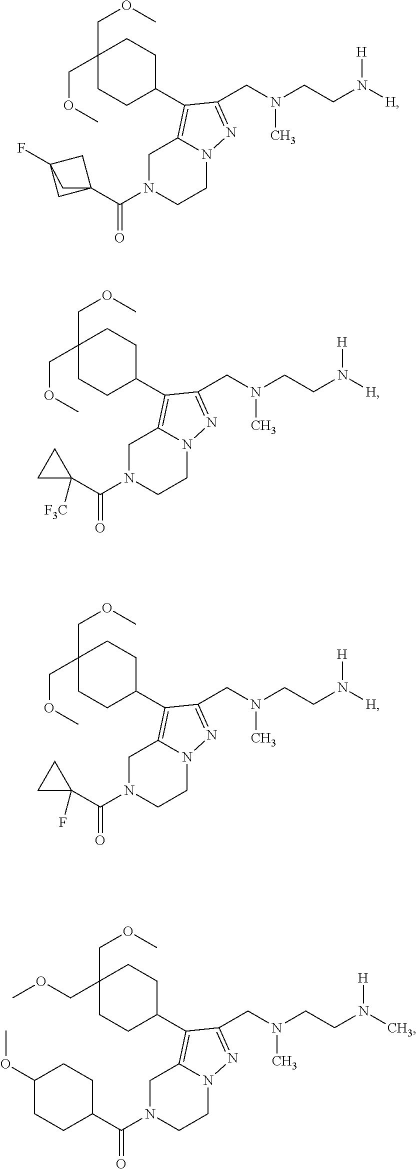

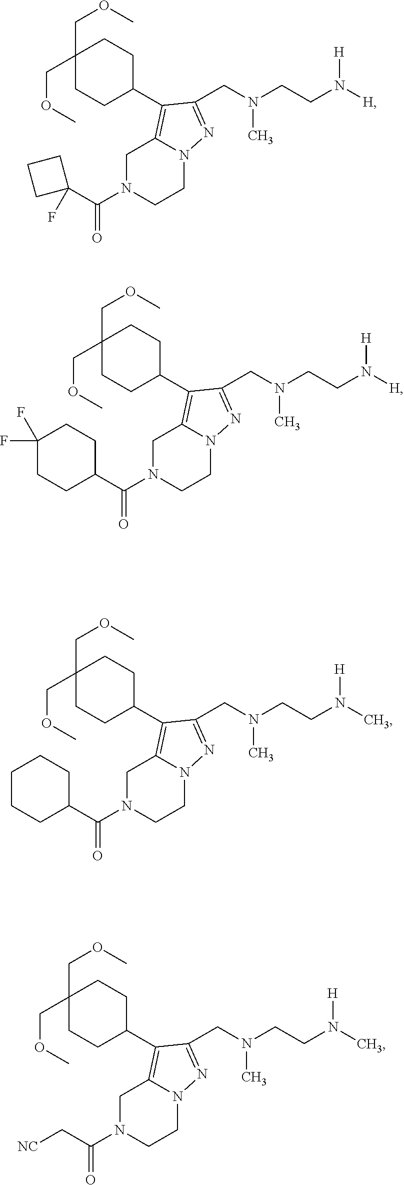

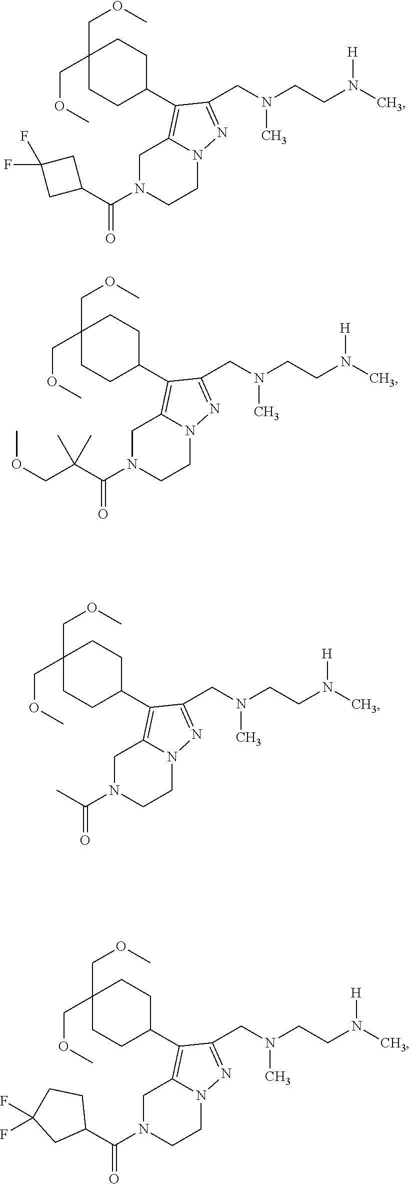

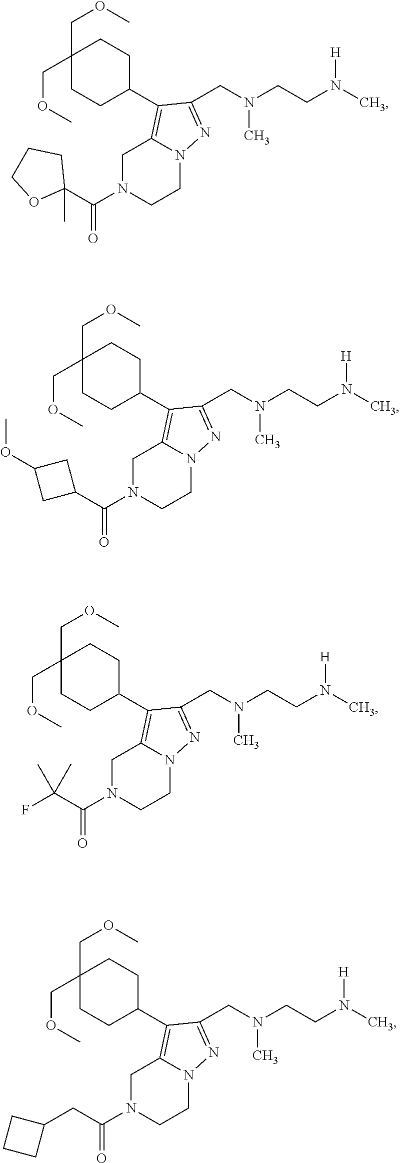 [SMILES: CNCCN(C)Cc1nn2c(c1C1CCC(COC)(COC)CC1)CN(C(=O)C1(C)CCCO1)CC2 ; CNCCN(C)Cc1nn2c(c1C1CCC(COC)(COC)CC1)CN(C(=O)C1CC(OC)C1)CC2 ; CNCCN(C)Cc1nn2c(c1C1CCC(COC)(COC)CC1)CN(C(=O)C(C)(C)F)CC2 ; CNCCN(C)Cc1nn2c(c1C1CCC(COC)(COC)CC1)CN(C(=O)CC1CCC1)CC2]

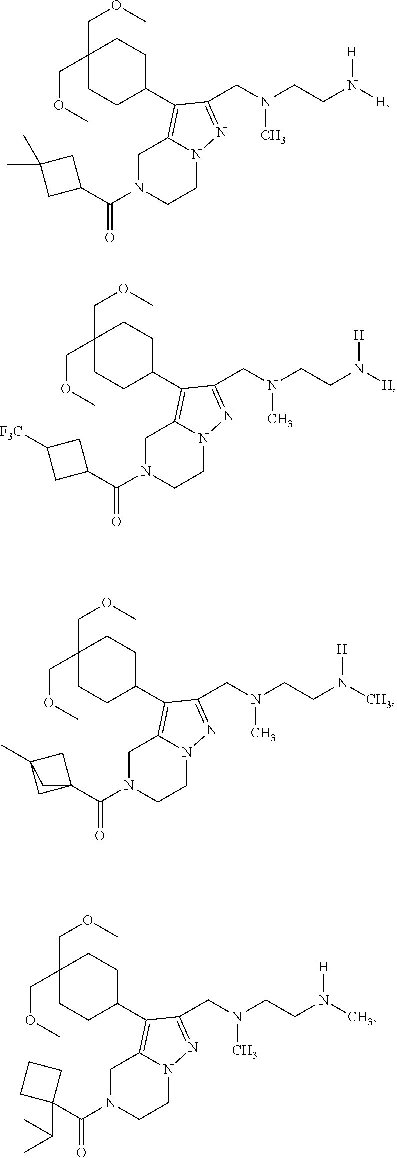

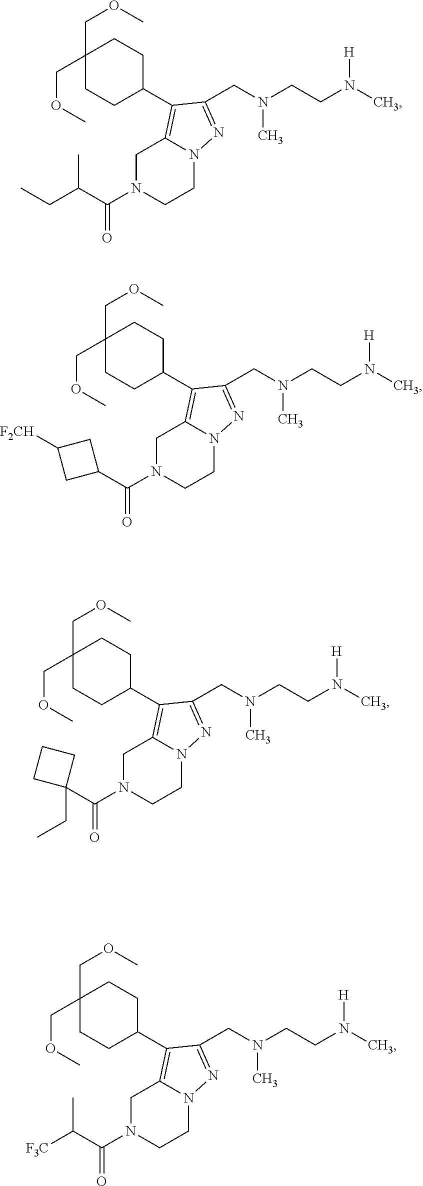

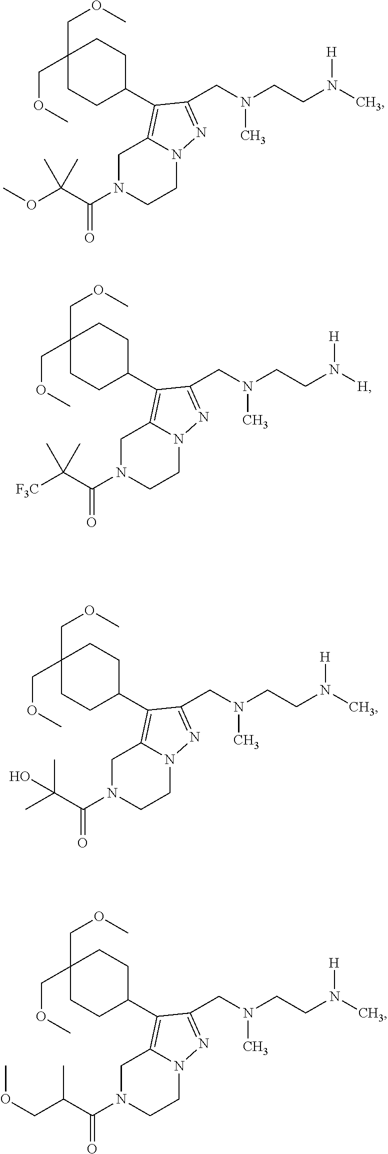

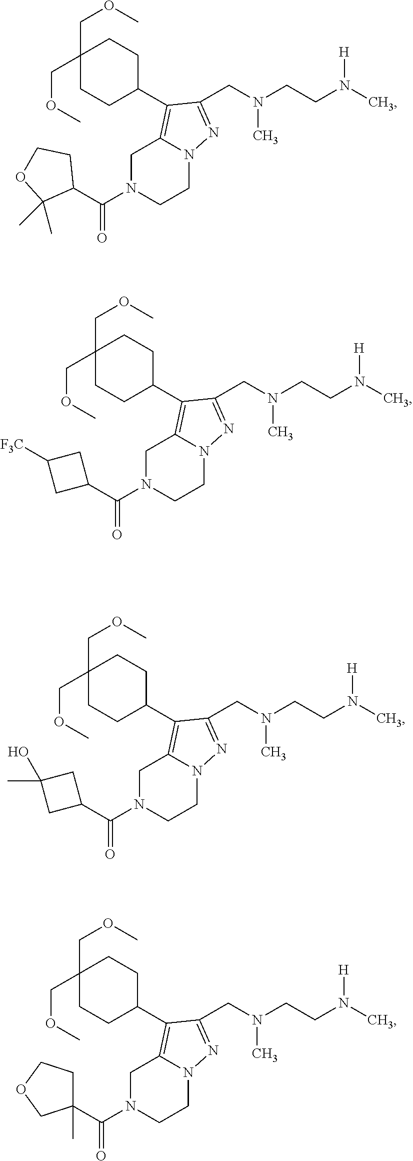 [SMILES: CNCCN(C)Cc1nn2c(c1C1CCC(COC)(COC)CC1)CN(C(=O)C1CCOC1(C)C)CC2 ; CNCCN(C)Cc1nn2c(c1C1CCC(COC)(COC)CC1)CN(C(=O)C1CC(C(F)(F)F)C1)CC2 ; CNCCN(C)Cc1nn2c(c1C1CCC(COC)(COC)CC1)CN(C(=O)C1CC(C)(O)C1)CC2 ; CNCCN(C)Cc1nn2c(c1C1CCC(COC)(COC)CC1)CN(C(=O)C1(C)CCOC1)CC2]

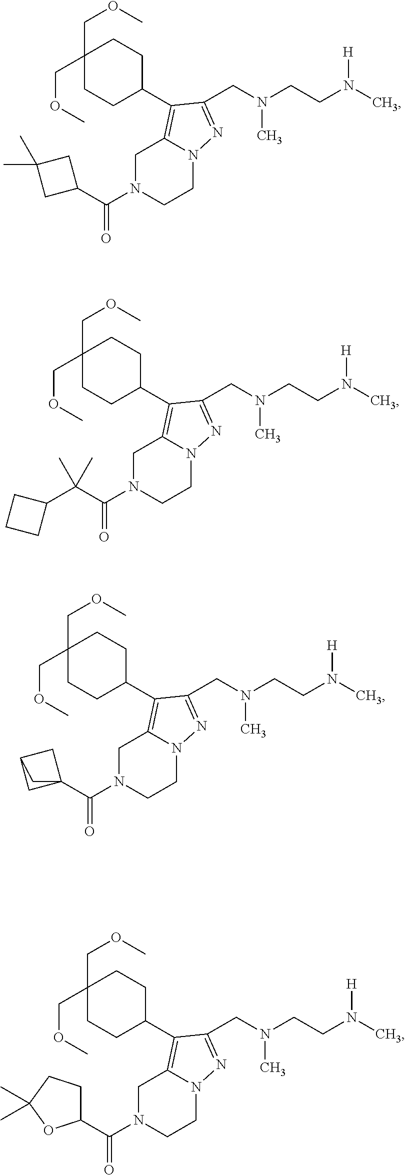 [SMILES: CNCCN(C)Cc1nn2c(c1C1CCC(COC)(COC)CC1)CN(C(=O)C1CC(C)(C)C1)CC2 ; CNCCN(C)Cc1nn2c(c1C1CCC(COC)(COC)CC1)CN(C(=O)C(C)(C)C1CCC1)CC2 ; CNCCN(C)Cc1nn2c(c1C1CCC(COC)(COC)CC1)CN(C(=O)C13CC(C1)C3)CC2 ; CNCCN(C)Cc1nn2c(c1C1CCC(COC)(COC)CC1)CN(C(=O)C1CCC(C)(C)O1)CC2]

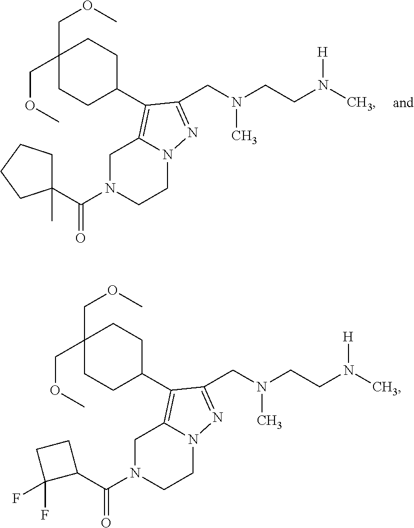 [SMILES: CNCCN(C)Cc1nn2c(c1C1CCC(COC)(COC)CC1)CN(C(=O)C1(C)CCCC1)CC2 ; CNCCN(C)Cc1nn2c(c1C1CCC(COC)(COC)CC1)CN(C(=O)C1CCC1(F)F)CC2]

or a salt thereof.

Also provided is a compound of structural Formula I, or a salt thereof, for use as a medicament.

Also provided is a compound of structural Formula I, or a salt thereof, for use in the treatment of cancer.

Also provided is a compound of structural Formula I, or a salt thereof, for use in the manufacture of a medicament for the prevention or treatment of a disease or condition ameliorated by the inhibition of PRMT.

Also provided is a pharmaceutical composition comprising a compound of structural Formula I, or a salt thereof, with a pharmaceutically acceptable carrier.

Also provided is a method of inhibition of a PRMT comprising contacting PRMT with a compound of structural Formula I, or a salt thereof. In some embodiments, the PRMT is PRMT1.

Also provided is a method of modulating gene expression comprising contacting a cell with an effective dose of the compound of structural Formula I or a salt thereof.

Also provided is a method of treatment of a PRMT-mediated disease comprising the administration of a therapeutically effective amount of a compound of structural Formula I, or a salt thereof, to a patient in need thereof. In certain embodiments, the PRMT-mediated disease is an autoimmune disease. In certain embodiments, the PRMT-mediated disease is amyotrophic lateral sclerosis. In certain embodiments, the PRMT-mediated disease is a muscular dystrophy. In certain embodiments, the PRMT-mediated disease is a vascular disease. In certain embodiments, the PRMT-mediated disease is a metabolic disorder. In certain embodiments, the PRMT-mediated disease is diabetes. In certain embodiments, the PRMT-mediated disease is a skeletal muscle metabolic disorder. In some embodiments, the disease is a proliferative disease. In some further embodiments, the proliferative disease is cancer.

Also provided is a method of treatment of cancer comprising the administration of a therapeutically effective amount of a compound of structural Formula I, or a salt thereof, to a patient in need thereof, wherein the cancer is chosen from acoustic neuroma, acute leukemia, acute lymphocytic leukemia, acute myelocytic leukemia, acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

Also provided is a method of treatment of cancer comprising the administration of a therapeutically effective amount of a compound of structural Formula I, or a salt thereof, to a patient in need thereof, and further comprising the administration of a non-chemical method of cancer treatment. In certain embodiments, the non-chemical method of cancer treatment is chosen from surgery, radiation therapy, thermoablation, focused ultrasound therapy, and cryotherapy.

Also provided is a method of treatment of cancer comprising the administration of a therapeutically effective amount of:
a. a compound of structural Formula I, or a salt thereof; and
b. another therapeutic agent,
   to a patient in need thereof.

Also provided is a method of treatment of cancer comprising the administration of a therapeutically effective amount of:
a. a compound of structural Formula I, or a salt thereof; and
b. a cytotoxic agent,
   to a patient in need thereof.

Also provided is a method of treatment of cancer comprising the administration of a therapeutically effective amount of:
a. a compound of structural Formula I, or a salt thereof; and
b. a cytotoxic agent chosen from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism,
   to a patient in need thereof.

Also provided are embodiments, wherein any embodiment above may be combined with any one or more of these embodiments, provided the combination is not mutually exclusive.

As used herein, two embodiments are “mutually exclusive” when one is defined to be something which is different than the other. For example, an embodiment, wherein two groups combine to form a cycloalkyl is mutually exclusive with an embodiment in which one group is ethyl the other group is hydrogen. Similarly, an embodiment, wherein one group is —CH₂— is mutually exclusive with an embodiment, wherein the same group is —NH—.

Also provided is a compound chosen from the Examples disclosed herein, or a salt thereof.

Also provided a method of inhibiting at least one PRMT function comprising the step of contacting PRMT with a compound as disclosed herein, or a salt thereof. The cell phenotype, cell proliferation, activity of PRMT, change in biochemical output produced by active PRMT, expression of PRMT, or binding of PRMT with a natural binding partner may be monitored. Such methods may be modes of treatment of disease, biological assays, cellular assays, biochemical assays, or the like.

Also provided herein is a method of treatment of a PRMT-mediated disease comprising the administration of a therapeutically effective amount of a compound as disclosed herein, or a salt thereof, to a patient in need thereof.

In certain embodiments, the disease is chosen from a vascular disease, a metabolic disease, an autoimmune disease, and a proliferative disease.

Also provided herein is a compound as disclosed herein, or a salt thereof, for use as a medicament.

Also provided herein is a compound as disclosed herein, or a salt thereof, for use as a medicament for the treatment of a PRMT-mediated disease.

Also provided is the use of a compound as disclosed herein, or a salt thereof, as a medicament.

Also provided is the use of a compound as disclosed herein, or a salt thereof, as a medicament for the treatment of a PRMT-mediated disease.

Also provided is a compound as disclosed herein, or a salt thereof, for use in the manufacture of a medicament for the treatment of a PRMT-mediated disease.

Also provided is the use of a compound as disclosed herein, or a salt thereof, for the treatment of a PRMT-mediated disease.

Also provided herein is a method of inhibition of type I PRMTs comprising contacting PRMT with a compound as disclosed herein, or a salt thereof.

Also provided herein is a method for achieving an effect in a patient comprising the administration of a therapeutically effective amount of a compound as disclosed herein, or a salt thereof, to a patient, wherein the effect is chosen from cognition enhancement.

Compounds disclosed herein, or salts thereof, may be selective amongst the PRMT isoforms, e.g. PRMT1, PRMT3, CARM1, PRMT6, and PRMT8 in various ways. For example, compounds disclosed herein, or salts thereof, may be selective for PRMT1 and/or PRMT6 over the other isoforms, be a pan-inhibitor of all the isoforms, or be selective for only one isoform. In certain embodiments, compounds, or salts thereof, are selective for PRMT1 over other isoforms.

In certain embodiments, the PRMT-mediated disease is chosen from vascular disease, a metabolic disease, an autoimmune disease, and a proliferative disease.

Also provided is a method of modulation of a PRMT-mediated function in a subject comprising the administration of a therapeutically effective amount of a compound as disclosed herein, or a salt thereof.

Also provided is a pharmaceutical composition comprising a compound as disclosed herein, or a salt thereof, together with a pharmaceutically acceptable carrier.

In certain embodiments, the pharmaceutical composition is formulated for oral administration.

In certain embodiments, the pharmaceutical composition is formulated for parenteral administration.

In certain embodiments, the pharmaceutical composition is formulated for intravenous administration.

In certain embodiments, the pharmaceutical composition is formulated for subcutaneous or intramuscular administration.

In certain embodiments, the oral pharmaceutical composition is chosen from a tablet and a capsule.

### Abbreviations and Definitions

As used herein, the terms below have the meanings indicated.

When ranges of values are disclosed, and the notation “from n1 . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings, wherein such polycyclic ring systems are fused together. The term “aryl” embraces aromatic groups such as phenyl, naphthyl, anthracenyl, and phenanthryl.

The terms “benzo” and “benz,” as used herein, alone or in combination, refer to the divalent group C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group, wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1.1.1]pentane, camphor, adamantane, and bicyclo[3.2.1]octane

The term “bicycloalkyl”, as used herein, alone or in combination, refers to a cyclic alkyl system that is characterized by the presence of two atoms, termed “bridgehead atoms” that are connected to each other via three bond pathways. “Bicycloalkyl” thus encompasses, by way of example, bicyclo[2.2.1]heptane, also known as norbornane, bicyclo[2.2.2]octane, bicyclo[2.2.0]hexane and bicyclo[3.3.0]octane.

The term “cycloalkoxy”, as used herein, alone or in combination, refers to a saturated, or partially unsaturated monocyclic, bicyclic, or tricyclic heterocyclic group containing at least one oxygen as a ring member. In some embodiments, said cycloalkoxy comprises 1, 2, or 3 heteroatoms as ring members. In some embodiments, said cycloalkoxy contains 1 or 2 heteroatoms as ring members. In some embodiments, said cycloalkoxy contains 1 oxygen as a ring member. In some embodiments, the heteroatoms in said heterocycloalkyl are independently chosen from nitrogen, oxygen, and sulfur. In some embodiments, the heteroatoms in said heterocycloalkyl are independently chosen from nitrogen and oxygen. In some embodiments, the heteroatoms in heterocycloalkyl are oxygen. In some embodiments, the heterocycloalkyl contains at least one aryl or heteroaryl ring. In some embodiments, the heterocycloalkyl does not contain either an aryl ring or a heteroaryl ring. Examples of cycloalkoxy groups include ethylene oxide, oxetane, tetrahydrofuran, 2,3-dihydrobenzofuran, dioxane, and morpholine.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “fluoroalkyl,” as used herein, alone or in combination, refers to an alkyl group having the meaning as defined above, wherein one or more hydrogens are replaced with a fluorine. Specifically embraced are monofluoroalkyl, difluoroalkyl and polyfluoroalkyl groups. Examples of fluoroalkyl groups include fluoromethyl, difluoromethyl, trifluoromethyl, 2,2,2-trifluoroethyl, pentafluoroethyl, heptafluoropropyl, difluoroethyl, difluoropropyl.

The term “halo,” or “halogen,” as used herein, alone or in combination, refers to fluorine, chlorine, bromine, or iodine.

The term “haloalkyl,” as used herein, alone or in combination, refers to an alkyl group having the meaning as defined above, wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups, wherein heterocyclic rings are fused with aryl rings, wherein heteroaryl rings are fused with other heteroaryl rings, wherein heteroaryl rings are fused with heterocycloalkyl rings, or, wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzoxazolyl, benzoxadiazolyl, benzothiazolyl, benzothiadiazolyl, benzofuryl, benzothienyl, chromonyl, coumarinyl, benzopyranyl, tetrazolopyridazinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. The term “heterocycloalkyl”, as used herein, alone or in combination, is understood to encompass “heterobicycloalkyl”, as defined below. The term “heterocycloalkyl”, as used herein, alone or in combination, is understood to encompass “lactone”, as defined below. The term “heterocycloalkyl”, as used herein, alone or in combination, is understood to encompass “lactam”, as defined below.

The term “hydroxy,” as used herein, alone or in combination, refers to —OH.

The terms “oxy” or “oxa,” as used herein, alone or in combination, refer to —O—.

The term “oxo,” as used herein, alone or in combination, refers to ═O.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group. Parentheses may be used to further clarify connectivity. For example, the terms “arylalkyl” and “(aryl)alkyl” are equivalent, and both refer to an aryl group attached to the parent molecule through an alkyl group. The term “(alkyl)aryl” refers to an alkyl group attached to the parent molecule through an aryl group, and may be described equivalently as an alkyl substituted aryl group. Similarly, the term “((alkyl)aryl)alkyl” refers to an ((alkyl)aryl) group attached to the parent molecule through an alkyl group, or equivalently as an alkyl substituted aryl group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently chosen from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PRMT1 inhibitor” is used herein to refer to a compound, or a salt thereof, that exhibits an IC50 with respect to PRMT1 activity of no more than about 100 μM and more typically not more than about 50 μM, as measured in the PRMT1 enzymatic assay described generally herein. “IC50” is that concentration of inhibitor which reduces the activity of an enzyme (e.g., PRMT1) to half-maximal level. Certain compounds disclosed herein, or salts thereof, have been discovered to exhibit inhibitory activity against PRMT1. In certain embodiments, compounds, or salts thereof, will exhibit an IC50 with respect to PRMT1 of no more than about 10 μM; in further embodiments, compounds, or salts thereof, will exhibit an IC50 with respect to PRMT1 of no more than about 2 μM; in yet further embodiments, compounds, or salts thereof, will exhibit an IC50 with respect to PRMT1 of not more than about 500 nM; in yet further embodiments, compounds, or salts thereof, will exhibit an IC50 with respect to PRMT1 of not more than about 200 nM; in yet further embodiments, compounds, or salts thereof, will exhibit an IC50 with respect to PRMT1 of not more than about 50 nM, as measured in the PRMT1 assay described herein.

The phrase “therapeutically effective” is intended to qualify the amount of active ingredients used in the treatment of a disease or disorder or on the effecting of a clinical endpoint.

The term “pharmaceutically acceptable” refers to those compounds, or salts thereof, which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The compounds disclosed herein can exist as salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, also provided are sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

### Pharmaceutical Compositions

While it may be possible for the compounds disclosed herein, or salts thereof, to be administered as the raw chemical, it is also possible to present them as a pharmaceutical formulation. Accordingly, provided herein are pharmaceutical formulations which comprise one or more of certain compounds disclosed herein, or one or more salts thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### Oral Administration

The compounds disclosed herein, or salts thereof, may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules, solutions or suspensions in an aqueous liquid or a non-aqueous liquid, or as an oil-in-water liquid emulsion or a water-in-oil liquid emulsion. The active ingredient may also be presented as a bolus, electuary or paste.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of active compound doses.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound of Formula (I) or a pharmaceutically acceptable salt thereof, and a pharmaceutically acceptable carrier.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds, or salts thereof, may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

### Parenteral Administration

Compounds disclosed herein, or salts thereof, may be administered directly into the blood stream, muscle, or internal organs by injection, e.g., by bolus injection or continuous infusion. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions. Compositions for parenteral administration may be formulated as immediate or modified release, including delayed or sustained release. Compounds, or salts thereof, may also be formulated as depot preparations. Such long acting formulations may be administered by implantation (for example subcutaneously or intramuscularly) or by intramuscular injection. Thus, for example, the compounds, or salts thereof, may be formulated with suitable polymeric or hydrophobic materials (for example as an emulsion in an acceptable oil) or ion exchange resins, or as sparingly soluble derivatives, for example, as a sparingly soluble salt.

The compounds disclosed herein, or salts thereof, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the compounds disclosed herein, or salts thereof, which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds, or salts thereof, to allow for the preparation of highly concentrated solutions.

In addition to the formulations described previously, the compounds disclosed herein, or salts thereof, may also be formulated as a depot preparation. Such long acting formulations may be administered by implantation (for example subcutaneously or intramuscularly) or by intramuscular injection. Thus, for example, the compounds, or salts thereof, may be formulated with suitable polymeric or hydrophobic materials (for example as an emulsion in an acceptable oil) or ion exchange resins, or as sparingly soluble derivatives, for example, as a sparingly soluble salt.

### Topical Administration

Compounds disclosed herein, or salts thereof, may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

Certain compounds disclosed herein, or salts thereof, may be administered topically, that is by non-systemic administration. This includes the application of a compound disclosed herein, or a salt thereof, externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

### Rectal, Buccal, and Sublingual Administration

Suppositories for rectal administration of the compounds disclosed herein, or salts thereof, can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds disclosed herein, or salts thereof, may also be formulated in rectal compositions such as suppositories or retention enemas, e.g., containing conventional suppository bases such as cocoa butter, polyethylene glycol, or other glycerides.

### Administration by Inhalation

For administration by inhalation, compounds disclosed herein, or salts thereof, may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds disclosed herein, or salts thereof, may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations disclosed herein may include other agents conventional in the art having regard to the type of formulation in question, for example those suitable for oral administration may include flavoring agents.

Compounds disclosed herein, or salts thereof, may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds disclosed herein, or a salt or salts thereof, which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds, or salts thereof, can be administered in various modes, e.g. orally, topically, or by injection. The precise amount of compound, or a salt thereof, administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

It should be understood that in addition to the ingredients particularly mentioned above, the formulations disclosed herein may include other agents conventional in the art having regard to the type of formulation in question, for example those suitable for oral administration may include flavoring agents.

Compounds, or salts thereof, may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds, or a salt or salts thereof, which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds, or salts thereof, can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### Combinations and Combination Therapy

In certain instances, it may be appropriate to administer at least one of the compounds disclosed herein, or a salt or salts thereof, in combination with another therapeutic agent. By way of example only, if one of the side effects experienced by a patient upon receiving one of the compounds disclosed herein, or a salt thereof, is hypertension, then it may be appropriate to administer an anti-hypertensive agent in combination with the initial therapeutic agent. Or, by way of example only, the therapeutic effectiveness of one of the compounds disclosed herein, or a salt thereof, may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds disclosed herein, or a salt thereof, with another therapeutic agent (which also includes a therapeutic regimen) that also has therapeutic benefit. By way of example only, in a treatment for diabetes involving administration of one of the compounds disclosed herein, or a salt thereof, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds disclosed herein, or salts thereof, with anti-cancer (chemotherapeutic) drugs. Classes of anti-cancer drugs include, but are not limited to: alkylating agents, anti-metabolites, antimitotics, checkpoint inhibitors, plant alkaloids and terpenoids, topoisomerase inhibitors, cytotoxic antibiotics, aromatase inhibitors, angiogenesis inhibitors, anti-steroids and anti-androgens, mTOR inhibitors, tyrosine kinase inhibitors, and others.

For use in cancer and neoplastic diseases a PRMT inhibitor may be optimally used together with one or more of the following non-limiting examples of anti-cancer agents:
(1) alkylating agents, including but not limited to carmustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, and cyclophosphamide (ENDOXAN);
(2) anti-metabolites, including but not limited to cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), raltitrexed;
(3) antimitotics, which are often plant alkaloids and terpenoids, or derivatives thereof, including but not limited to taxanes such as docetaxel (TAXITERE) and paclitaxel (ABRAXANE, TAXOL); vinca alkaloids such as vincristine (ONCOVIN), vinblastine, vindesine, and vinorelbine (NAVELBINE);
(4) checkpoint inhibitors, such as anti-PD-1 or PD-L1 antibodies pembrolizumab (KEYTRUDA), nivolumab (OPDIVO), MEDI4736, and MPDL3280A; anti-CTLA-4 antibody ipilimumab (YERVOY); and those that target LAG3 (lymphocyte activation gene 3 protein), KIR (killer cell immunoglobulin-like receptor), 4-1BB (tumour necrosis factor receptor superfamily member 9), TIM3 (T-cell immunoglobulin and mucin-domain containing-3) and OX40 (tumour necrosis factor receptor superfamily member 4);
(5) topoisomerase inhibitors, including but not limited to camptothecin (CTP), irinotecan (CAMPTOSAR), topotecan (HYCAMTIN), teniposide (VUMON), and etoposide (EPOSIN);
(6) cytotoxic antibiotics, including but not limited to actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOVANTRONE), plicamycin;
(7) aromatase inhibitors, including but not limited to aminoglutethimide, anastrozole (ARIMIDEX), letrozole (FEMARA), vorozole (RIVIZOR), exemestane (AROMASIN);
(8) angiogenesis inhibitors, including but not limited to genistein, sunitinib (SUTENT) and bevacizumab (AVASTIN);
(9) anti-steroids and anti-androgens such as aminoglutethimide (CYTADREN), bicalutamide (CASODEX), cyproterone, flutamide (EULEXIN), nilutamide (NILANDRON);
(10) tyrosine kinase inhibitors, including but not limited to imatinib (GLEEVEC), erlotinib (TARCEVA), lapatininb (TYKERB), sorafenib (NEXAVAR), and axitinib (INLYTA);
(11) mTOR inhibitors such as everolimus, temsirolimus (TORISEL), and sirolimus;
(12) monoclonal antibodies such as trastuzumab (HERCEPTIN) and rituximab (RITUXAN);
(13) other agents, such as amsacrine; Bacillus Calmette-Gudrin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

In any case, the multiple therapeutic agents (at least one of which is a compound disclosed herein, or a salt or salts thereof) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating PRMT-mediated disorders in a human or animal subject in need of such treatment comprising administering to said subject an amount of a compound disclosed herein, or a salt thereof, effective to reduce or prevent said disorder in the subject, in combination with at least one additional agent for the treatment of said disorder that is known in the art. In a related aspect, certain embodiments provide therapeutic compositions comprising at least one compound disclosed herein, or a salt or salts thereof, in combination with one or more additional agents for the treatment of PRMT-mediated disorders.

Specific diseases to be treated by the compounds, salts, compositions, and methods disclosed herein include proliferative diseases, neurological diseases, amyotrophic lateral sclerosis, muscular dystrophies, autoimmune disorders, vascular disorders, and metabolic disorders.

Besides being useful for human treatment, certain compounds, salts, and formulations disclosed herein may also be useful for veterinary treatment of companion animals, exotic animals and farm animals, including mammals, rodents, and the like. More preferred animals include horses, dogs, and cats.

### Compound Synthesis

Compounds of the present disclosure, and salts thereof, can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure, and salts thereof, are commercially available or can be prepared using routine methods known in the art.

### LIST OF ABBREVIATIONS

Ac₂O=acetic anhydride; AcCl=acetyl chloride; AcOH=acetic acid; AIBN=azobisisobutyronitrile; aq. =aqueous; BPD=bis(pinacolato)diboron=,4,5,5-tetramethyl-2-(4,4,5,5-tetramethyl-1,3,2-dioxaborolan-2-yl)-1,3,2-dioxaborolane; Bu₃SnH=tributyltin hydride; CD₃OD=deuterated methanol; CDCl₃=deuterated chloroform; CDI=1,1′-Carbonyldiimidazole; DAST=diethylaminosulfur trifluoride; DBU=1,8-diazabicyclo[5.4.0]undec-7-ene; DCE=1,2-dichloroethane; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMP=Dess-Martin periodinane; DMSO-d₆=deuterated dimethyl sulfoxide; DMSO=dimethyl sulfoxide; DPPA=diphenylphosphoryl azide; EDC·HCl=EDCI·HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; IBX=2-iodoxybenzoic acid; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PE=petroleum ether; PG=protecting group; prep-HPLC=preparative high-performance liquid chromatography; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat. =saturated; ss=saturated solution; t-BuOH=tert-butanol; T3P=Propylphosphonic Anhydride; TBS=TBDMS=tert-butyldimethylsilyl; TBSCl=TBDMSCI=tert-butyldimethylchlorosilane; TEA=Et₃N=triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

### General Synthetic Methods for Preparing Compounds

The following schemes can be used to synthesize the compounds of this disclosure.

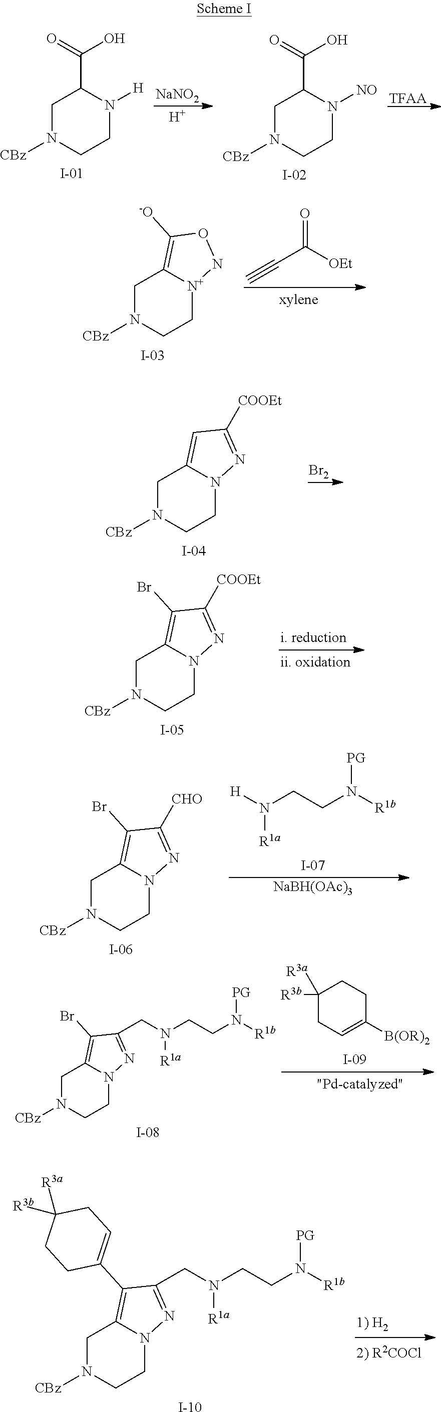

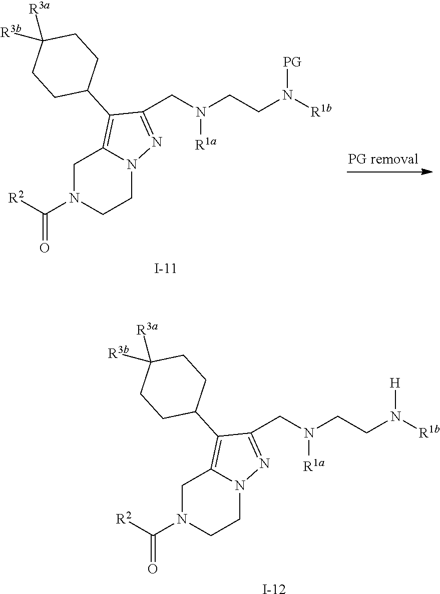

Bicyclic-pyrazole compounds with a functionalized amino side-chain of general formula I-12 can be prepared as shown in Scheme I. Nitration of protected piperidine 2-carboxylic acid I-01 can be performed in the presence of sodium nitrite in aqueous acid to give a corresponding nitroso intermediate I-02. Treatment of the latter with a dehydrating agent, such as TFAA, in an appropriate solvent provides a zwitterionic intermediate of general formula I-03. Dipolar cycloaddition of intermediate I-03 with a suitable propiolate (e.g. ethyl propiolate) at elevated temperatures can yield the corresponding functionalized bicyclic pyrazole ester I-04. Bromination of this bicyclic pyrazole intermediate with a bromination reagent (e.g. elemental bromine) in an appropriate organic solvent gives the bromo-ester of general formula I-05. The corresponding bicyclic pyrazole carboxaldehyde I-06 can be obtained from I-05 by reduction to the hydroxymethyl derivative (e.g. with lithium aluminium hydride) and subsequent reaction with an oxidizing agent (e.g. Des-Martin periodinane) in the appropriate organic solvent. Installation of the amino side chain to the bicyclic pyrazole aldehyde can be accomplished through reductive amination with a suitably protected ethylendiamine derivative I-07 (e.g. PG can be Boc or Fmoc). The amino side chain of interest can be reacted with the aldehyde of the bicyclic pyrazole in the presence of a reducing agent such as sodium triacetoxy borohydride to give the intermediate of general formula I-08. A palladium mediated coupling can be utilized to install a highly functionalized cyclic alkene by reacting I-08 with the boronic ester or acid I-09 in the presence of a suitable catalyst (e.g. PdCl₂(dppf)) and a suitable base (e.g. K₂CO₃) with an appropriate organic solvent (e.g. dioxane) at elevated temperature, yielding a di-substituted bicyclic pyrazole of general structure I-10. Functional bicyclic pyrazole I-12 can then be derived from I-10 by reduction of the olefin with a suitable metal catalyst (e.g. palladium on carbon) under an atmosphere of hydrogen to give I-11, followed by removal of the amino side chain protecting group.

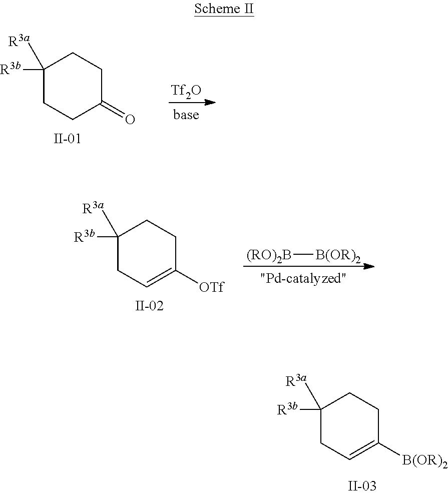

Boronic acids or esters of general structure II-03 can be commercially available or can be prepared in a straightforward manner starting from the corresponding alkenyl bromides using a strong base such as BuLi, and reacting the resulting highly reactive lithium species with trimethyl borate. Alternatively, boronic acids or esters I-e can be prepared by the corresponding ketone II-01, as described in Scheme II. Ketone II-01 can be converted to enol triflate II-02 and further functionalized into boronate II-03 via palladium mediated coupling reactions with suitable boron derivatives.

This disclosure contemplates the use of selective hydrogenation methods to enrich a reduction product mixture in a certain diastereomer. Many methods are available for diastereoselective reduction, including the use of modified transition metal catalysts, and enantiopure ligands for metal catalysts.

In certain embodiments, compounds of Formula (II) or (III) have substituted cyclohexene and cyclohexane moieties that are related to the structures depicted for I-10 and I-11, respectively, in Scheme I. This disclosure contemplates diastereomeric mixtures as well as compounds that have been separated into individual diastereomers.

In certain embodiments, the compounds, or salts thereof, are provided in an approximately 1:1 ratio of diastereomers. In certain embodiments, the compounds, or salts thereof, are provided in a 20% or greater diastereomeric excess of one diastereomer. In certain embodiments, the compounds, or salts thereof, are provided in a 50% or greater diastereomeric excess of one diastereomer. In certain embodiments, the compounds, or salts thereof, are provided in a 80% or greater diastereomeric excess of one diastereomer. In certain embodiments, the compounds, or salts thereof, are provided in a 90% or greater diastereomeric excess of one diastereomer. In certain embodiments, the compounds, or salts thereof, are provided in a 95% or greater diastereomeric excess of one diastereomer. In certain embodiments, the compounds, or salts thereof, are provided in a 99% or greater diastereomeric excess of one diastereomer. In certain embodiments, the compounds, or salts thereof, are provided in essentially diastereomerically pure form.

The following Intermediates are used to synthesize the Example compounds disclosed herein.

### Intermediate a

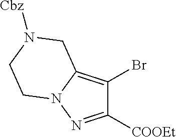 [SMILES: CCOC(=O)c1nn2c(c1Br)CN(C(=O)OCc1ccccc1)CC2]

### 5-Benzyl 2-ethyl 3-bromo-6,7-dihydropyrazolo[1,5-a]pyrazine-2,5(4H)-dicarboxylate

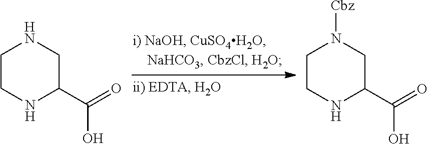 [SMILES: O=C(O)C1CNCCN1 ; O=C(O)C1CN(C(=O)OCc2ccccc2)CCN1]

4-((Benzyloxy)carbonyl)piperazine-2-carboxylic acid: To a solution of piperazine-2-carboxylic acid (290 g, 2.23 mol) in H₂O (1.16 L) at 20° C. were added aq. NaOH (2.5 M, 1.13 L, 2.82 mol), a solution of CuSO₄·5H₂O (188.5 g, 0.755 mol) in H₂O (2.32 L) and aq. NaHCO₃ (224.7 g, 104 mL, 2.68 mol). The resulting mixture was cooled to 0° C., a solution of CbzCl (266.1 g, 221.7 mL, 1.56 mol) in dioxane (1.16 L) was added dropwise over 10 minutes and the mixture was stirred at 20° C. for 16 hours. The suspension was filtered and the filter cake was dried under reduced pressure to give a light blue solid. The solid was added portionwise to a solution of EDTA (651.2 g, 1.36 L, 2.23 mol) in H₂O (2.32 L), and the suspension was stirred at 80° C. for 1 hour, filtered and the filter cake was washed with water to afford the title compound as a solution in water (500 g in 2.50 L) which was used into the next step directly.

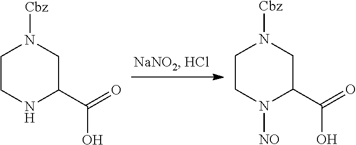

4-((Benzyloxy)carbonyl)-1-nitrosopiperazine-2-carboxylic acid: To a solution of the product from the previous step (500 g) in H₂O (2.5 L) at 0° C. were added aq. HCl (37% weight, 274.18 mL, 2.84 mol) followed by addition of NaNO₂ (195.8 g, 2.84 mol) portionwise, so to maintain the temperature of the reaction mixture in the 0-4° C. range, and the mixture was stirred at 0° C. for an additional 1 hour. The mixture was then extracted with EtOAc (5000 mL×2), the organic layers were combined, washed with brine (500 mL×3), dried over Na₂SO₄, filtered and concentrated under reduced pressure to afford the title compound as an oil (314 g) which was used without further purification.

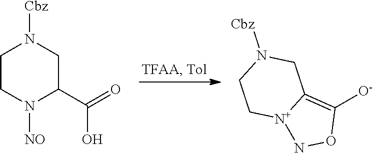

5-((Benzyloxy)carbonyl)-4,5,6,7-tetrahydro-[1,2,3]oxadiazolo[3,4-a]pyrazin-8-ium-3-olate: To a solution of the product from the previous step (314 g, 1.07 mol) in toluene (2.0 L) at 0° C. was added trifluoroacetic anhydride (337.3 g, 223.4 mL, 1.61 mol). The mixture was stirred at 20° C. for 16 hours and then concentrated under reduced pressure. The residue was purified by column chromatography (SiO₂, Petroleum ether/EtOAc=20/1 to 1/1) to afford the title compound as a pale yellow oil (290 g, 956 mmol, 89.6%).

MS(ES⁺) C₁₃H₁₃N₃O₄ requires: 275, found 276 [M+H]⁺.

¹H NMR (400 MHz, CDCl₃) δ 7.44-7.31 (m, 5H), 5.21 (s, 2H), 4.61 (s, 2H), 4.45-4.27 (m, 2H), 4.03 (t, J=5.2 Hz, 2H).

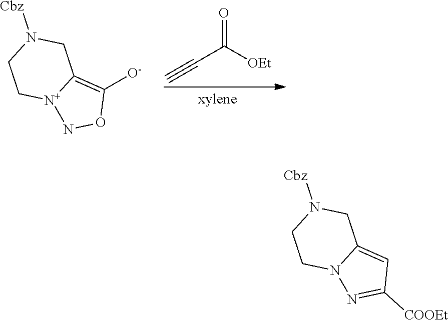

5-Benzyl 2-ethyl 6,7-dihydropyrazolo[1,5-a]pyrazine-2,5(4H)-dicarboxylate: To a solution of the product from the previous step (290 g, 1.05 mol) in xylene (100 mL) was added ethyl prop-2-ynoate (258.38 g, 258.38 mL, 2.63 mol), and the mixture was stirred at 130° C. for 16 hours. The reaction mixture was then cooled to RT and concentrated under reduced pressure. The residue was purified by column chromatography (SiO₂, Petroleum ether/EtOAc=10/1 to 2/1) to afford the title compound as a pale yellow oil (197 g, 598 mmol, 56.7% yield).

MS(ES⁺) C₁₇H₁₉N₃O₄ requires: 329, found 330 [M+H]+.

¹H NMR (400 MHz, CDCl₃) δ 7.38-7.31 (m, 5H), 6.60 (s, 1H), 5.12 (s, 2H), 4.74 (s, 2H), 4.38 (q, J=7.1 Hz, 2H), 4.33-4.19 (m, 2H), 3.97 (t, J=5.3 Hz, 2H), 1.38 (t, J=7.1 Hz, 3H).

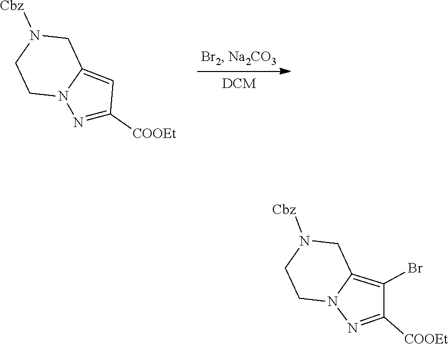

5-Benzyl 2-ethyl 3-bromo-6,7-dihydropyrazolo[1,5-a]pyrazine-2,5(4H)-dicarboxylate (Intermediate A): To a solution of Br₂ (121.1 g, 39.07 mL, 757.9 mmol) in CH₂Cl₂(2000 mL) at 0° C. were added Na₂CO₃ (123.6 g, 1.17 mol) and the product from the previous step (192 g, 582.9 mmol), and the mixture was stirred at 0° C. for 2 hours. The mixture was filtered, the filtrate was diluted with water (2000 mL) and extracted with CH₂Cl₂ (2000 mL×3). The combined organic layers were washed with brine (1000 mL×3), dried over Na₂SO₄, filtered and concentrated under reduced pressure to afford the title compound as an oil (230 g, 507 mmol, 86.9% yield).

MS(ES⁺) C₁₇H₁₈BrN₃O₄ requires: 407/409, found 408/410 [M+H]⁺.

¹H NMR (400 MHz, DMSO-d6) δ 7.47-7.30 (m, 5H), 5.17 (s, 2H), 4.58 (s, 2H), 4.29 (q, J=7.2 Hz, 2H), 4.23 (t, J=5.4 Hz, 2H), 4.02-3.87 (m, 2H), 1.29 (t, J=7.1 Hz, 3H).

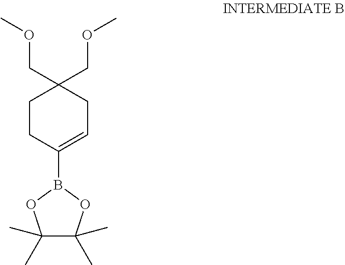

### 2-[4,4-Bis(methoxymethyl)cyclohexen-1-yl]-4,4,5,5-tetramethyl-1,3,2-dioxaborolane

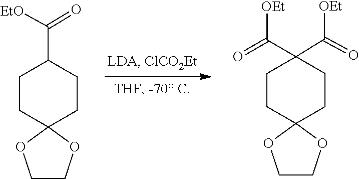 [SMILES: CCOC(=O)C1CCC2(CC1)OCCO2 ; CCOC(=O)C1(C(=O)OCC)CCC2(CC1)OCCO2]

Diethyl 1,4-dioxaspiro[4.5]decane-8,8-dicarboxylate. To a solution of ethyl 1,4-dioxaspiro[4.5]decane-8-carboxylate (10 g, 46.67 mmol) in THF (150 mL) at −70° C. was added dropwise LDA (2 M in THF, 25.67 mL, 1.10 eq), and the mixture was stirred at −70° C. for 1 hr. Ethyl chloroformate (5.32 g, 49.0 mmol, 4.67 mL, 1.05 eq) was then added dropwise, the mixture was slowly warmed to 15° C. and stirred for 11 hr. The reaction mixture was then poured into saturated aq. NH₄Cl (200 mL), the layers were separated, and the aqueous layer was extracted with EtOAc (50 mL×4). The combined organic layers were washed with brine (200 mL), dried over Na₂SO₄ and concentrated. The residue was purified by SiO₂ gel chromatography (PE to PE: EtOAc=20:1) to afford the title compound as a light yellow oil (9.50 g, 33 mmol, 71% yield). ¹H NMR (400 MHz, CDCl₃) δ 4.19 (q, J=7.2 Hz, 4H), 3.96-3.92 (m, 4H), 2.23-2.13 (m, 4H), 1.73-1.65 (m, 4H), 1.25 (t, J=7.2 Hz, 6H).

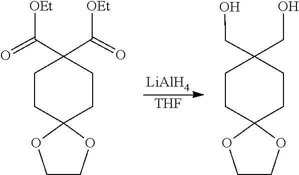

(1,4-Dioxaspiro[4.5]decane-8,8-diyl)dimethanol. To a suspension of LiAlH₄ (3.00 g, 79 mmol, 2.38 eq) in THF (100 mL) at 0° C. was added dropwise a solution of diethyl 1,4-dioxaspiro[4.5]decane-8,8-dicarboxylate (9.50 g, 33.2 mmol, 1.0 eq) in THF (50 mL). The resulting mixture was stirred for 1 hr at 15° C., then diluted with THF (200 mL), followed by dropwise addition of water (3 mL), 15% NaOH aqueous solution (3 mL) and water (9 mL). Anhydrous MgSO₄ was added and the mixture was stirred at RT for 0.5 h. The mixture was filtered through a pad of CELITE® and washed with EtOAc (100 mL). The filtrate was concentrated under reduced pressure to give the title compound as a white solid (6.20 g, 30.66 mmol, 92% yield) as white solid. ¹H NMR (400 MHz, CDCl₃) δ 4.30 (t, J=4.4 Hz, 2H), 3.82 (s, 4H), 3.27-3.21 (m, 4H), 1.49-1.46 (m, 4H), 1.42-1.36 (m, 4H).

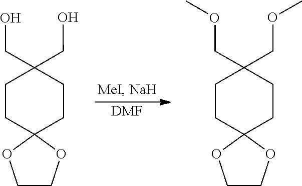

8,8-Bis(methoxymethyl)-1,4-dioxaspiro[4.5]decane. To a solution of [8-(hydroxymethyl)-1,4-dioxaspiro[4.5]decan-8-yl]methanol (6.20 g, 30.66 mmol) in DMF (70 mL) at 0° C. was added portionwise NaH (3.07 g, 76.65 mmol, 60% weight, 2.50 eq). The mixture was stirred at 0° C. for 1 h, Mel (11.4 g, 80.3 mmol, 5.0 mL, 2.62 eq) was added dropwise and the mixture was allowed to warm slowly to 15° C. while stirring for 15 hr. Saturated aq. NH₄Cl (100 mL) was added followed by water (200 mL). The mixture was extracted with EtOAc (150 mL×3), the combined organic layers were washed with brine (150 mL), dried over Na₂SO₄ and concentrated under reduced pressure. The residue was purified by SiO₂ gel chromatography (PE: EtOAc=100:1 to 30:1) to afford the title compound (5.0 g, 21.71 mmol, 71% yield) as colorless oil. ¹H NMR (400 MHz, CDCl₃) δ 3.93 (s, 4H), 3.30 (s, 6H), 3.25 (s, 4H), 1.63-1.60 (m, 4H), 1.55-1.52 (m, 4H).

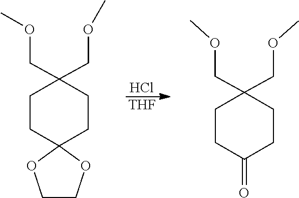

4,4-Bis(methoxymethyl)cyclohexanone. To a solution of 8,8-bis(methoxymethyl)-1,4-dioxaspiro[4.5]decane (5.0 g, 21.7 mmol) in THF (20 mL) at 15° C. was added HCl (4 M in dioxane, 20 mL, 3.68 eq). The resulting solution was stirred in a pressure safe closed reaction vessel at 40° C. for 12 hr, then cooled to RT and extracted with EtOAc (100 mL×3). The combined organic layers were washed with saturated aq NaHCO₃ (100 mL), brine (100 mL), dried (Na₂SO₄) and concentrated under reduced pressure to give 4,4-bis(methoxymethyl)cyclohexanone (3.50 g, 18.79 mmol, 86% yield) as pale yellow oil.

¹H NMR (400 MHz, CDCl₃) δ 3.35 (s, 6H), 3.33 (s, 4H), 2.36-2.32 (m, 4H), 1.79-1.75 (m, 4H).

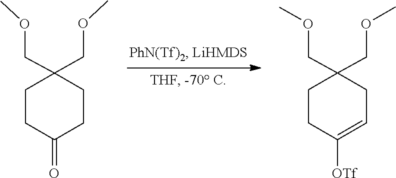

[4,4-Bis(methoxymethyl)cyclohexen-1-yl] trifluoromethanesulfonate. To a solution of 4,4-bis(methoxymethyl)cyclohexanone (1.50 g, 8.05 mmol) in THF (20 mL) at −70° C. was added dropwise LiHMDS (1 M in hexanes, 9.66 mL, 1.20 eq) and the mixture was stirred at −70° C. for 1 h. A solution of 1,1,1-trifluoro-N-phenyl-N-(trifluoromethylsulfonyl)methanesulfonamide (3.02 g, 8.45 mmol, 1.05 eq) in THF (10 mL) was added dropwise and the reaction mixture was slowly warmed to 15° C. while stirring for 15 hr. Saturated aq. NH₄Cl (80 mL) was added, the layers were separated and the aqueous layer was extracted with EtOAc (50 mL×4). The combined organic layers were washed with brine (100 mL), dried (Na₂SO₄) and concentrated under reduced pressure. The residue was purified by SiO₂ gel chromatography (PE to PE: EtOAc=100:1) to give the title compound (1.60 g, 5.03 mmol, 62% yield) as light yellow oil. ¹H NMR (400 MHz, CDCl₃) δ 5.69-5.67 (m, 1H), 3.35 (s, 6H), 3.25-3.19 (m, 4H), 2.31-2.29 (m, 2H), 2.09-2.06 (m, 2H), 1.74-1.69 (m, 2H).

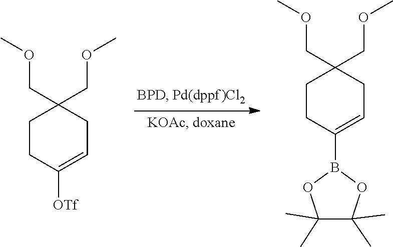

2-[4,4-Bis(methoxymethyl)cyclohexen-1-yl]-4,4,5,5-tetramethyl-1,3,2-dioxaborolane. A mixture of the product from the previous step (1.60 g, 5.03 mmol), BPD (1.53 g, 6.04 mmol, 1.20 eq), KOAc (987.29 mg, 10.06 mmol, 2.00 eq) and Pd(dppf)Cl₂ (184.03 mg, 251.50 μmol, 0.05 eq) in dioxane (30 mL) was degassed and purged with N₂ for three times, and heated at 90° C. for 16 hr. The reaction mixture was diluted with EtOAc (50 mL), filtered through a pad of CELITE® and the filtrate was concentrated under reduced pressure. The residue was purified by SiO₂ gel chromatography (PE to PE: EtOAc=100:1) to give the title compound (1.10 g, 3.71 mmol, 74% yield) as a pale yellow oil. ¹H NMR (400 MHz, CDCl₃) δ 6.49-6.48 (m, 1H), 3.34 (s, 6H), 3.25-3.17 (m, 4H), 2.11-2.09 (m, 2H), 1.97-1.95 (m, 2H), 1.50-1.47 (m, 2H), 1.26 (s, 12H).

### EXAMPLE 1

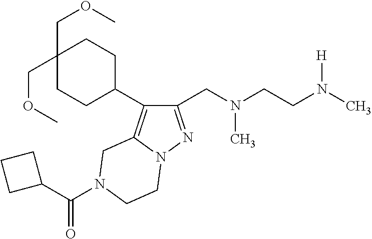 [SMILES: CNCCN(C)Cc1nn2c(c1C1CCC(COC)(COC)CC1)CN(C(=O)C1CCC1)CC2]

### (3-(4,4-Bis(methoxymethyl)cyclohexyl)-2-((methyl(2-(methylamino)ethyl)amino)methyl)-6,7-dihydropyrazolo[1,5-a]pyrazin-5(4H)-yl)(cyclobutyl)methanone

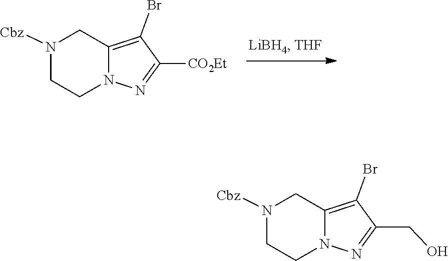 [SMILES: CCOC(=O)c1nn2c(c1Br)CN(C(=O)OCc1ccccc1)CC2 ; O=C(OCc1ccccc1)N1CCn2nc(CO)c(Br)c2C1]

Step 1: Benzyl 3-bromo-2-(hydroxymethyl)-6,7-dihydropyrazolo[1,5-a]pyrazine-5(4H)-carboxylate: A solution of Intermediate A (120 g, 293.9 mmol) in THF (600 mL) was added dropwise to a mixture of LiBH₄ (12.8 g, 587.9 mmol) in THF (800 mL) at 25° C. The resulting mixture was stirred at 45° C. for 2 hours, cooled to RT and poured into saturated aq. NH₄Cl (1500 mL) at 0° C. The mixture was extracted with EtOAc (400 mL×5). The combined organic layers were washed with brine (300 mL×3), dried over Na₂SO₄ and concentrated under reduced pressure to give the title compound as a pale yellow oil (109 g), which was used without further purification in the next step. MS (ES⁺) C₁₅H₁₆BrN₃O₃ requires: 365/367, found: 366/368. [M+H]⁺.

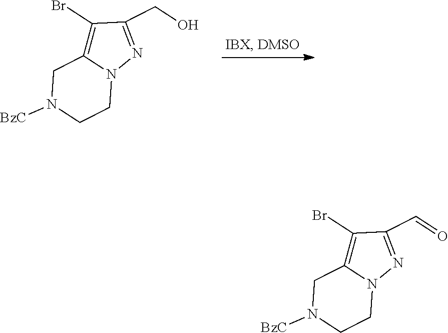

Step 2: Benzyl 3-bromo-2-formyl-6,7-dihydropyrazolo[1,5-a]pyrazine-5(4H)-carboxylate: To a solution of the product from the previous step (109 g, 297.6 mmol) in DMSO (1000 mL) at 30° C. was slowly added IBX (166.7 g, 595.3 mmol), so to keep the temperature of the reaction mixture within the 30-40° C. range. The resulting mixture was stirred at 30° C. for 2 hours, cooled to RT, poured into a mixture of water (1000 mL) and EtOAc (500 mL) at 0° C. and stirred for 30 minutes. The mixture was filtered, and the filtrate was extracted with EtOAc (400 mL×5). The combined organic layers were washed with brine (200 mL×3), dried over Na₂SO₄, filtered and concentrated under reduced pressure. The residue was purified by column chromatography (SiO₂, Petroleum ether/EtOAc=30/1 to 5/1) to give the title compound as yellow solid (107 g, 270.3 mmol, 90.8%).

MS (ES⁺) C₁₅H₁₄BrN₃O₃ requires: 363/365, found: 364/366. [M+H]⁺.

¹H NMR (400 MHz, CDCl3) δ 9.93 (s, 1H), 7.39-7.27 (m, 5H), 5.22 (s, 2H), 4.67 (s, 2H), 4.26 (s, 2H), 4.03-4.00 (m, 2H).

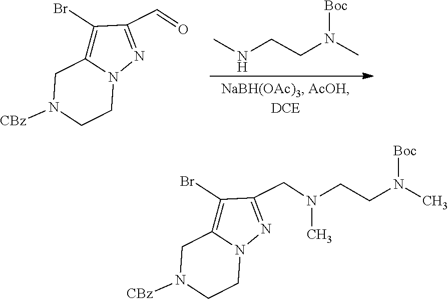

Step 3: Benzyl 3-bromo-2-(((2-((tert-butoxycarbonyl)(methyl)amino)ethyl)-(methyl)amino)methyl)-6,7-dihydropyrazolo[1,5-a]pyrazine-5(4H)-carboxylate: A mixture of the product from the previous step (107 g, 293.8 mmol) and tert-butyl N-methyl-N-[2-(methylamino)ethyl]carbamate (66.4 g, 352.6 mmol) in DCE (1000 mL) was stirred at RT for 20 minutes. HOAc (1.7 g, 1.68 mL, 29.4 mmol) was then added, followed by portionwise addition of NaBH(OAc)₃ (124.5 g, 587.6 mmol). The resulting mixture was stirred at for 12 hours, diluted with CH₂Cl₂ (500 mL), and a solution of saturated aq. NaHCO₃ (200 mL) was slowly added. The mixture was stirred for further 30 minutes, the layers were separated, and the organic phase was washed with brine (200 mL×2), dried over Na₂SO₄ and concentrated under reduced pressure. The residue was purified by column chromatography (SiO₂, Petroleum ether/EtOAc=30/1 to 5/1) to give the title compound as a pale yellow oil (137 g). MS (ES⁺) C₂₄H₃₄BrN₅O₄ requires: 535/537, found: 536/538 [M+H]⁺.

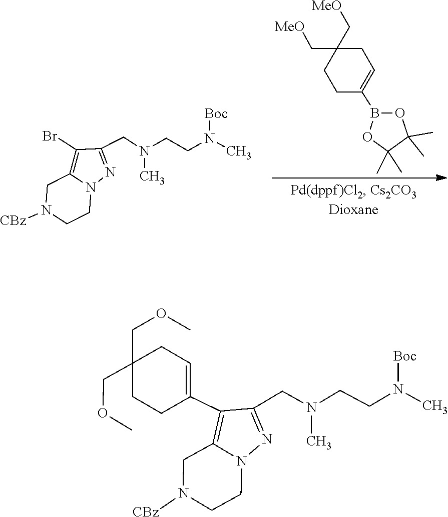

Step 4: Benzyl 3-(4,4-bis(methoxymethyl)cyclohex-1-en-1-yl)-2-(((2-((tert-butoxycarbonyl)(methyl)amino)ethyl)(methyl)amino)methyl)-6,7-dihydropyrazolo-[1,5-a]pyrazine-5(4H)-carboxylate: To a solution of the product from the previous step (89 g, 165.9 mmol) in a H₂O/dioxane mixture (1/10 v/v, 1100 mL) were added Intermediate B (68.8 g, 232.3 mmol) and Cs₂CO₃ (162.2 g, 497.7 mmol). The reaction vessel was purged with N₂ (3×), Pd(dppf)Cl₂ (6.07 g, 8.3 mmol) was added, and the mixture was stirred at 90° C. for 12 hours. The reaction mixture was then cooled to RT, diluted with EtOAc (500 mL), dried over Na₂SO₄ and filtered through a pad of CELITE®. The filtrate was concentrated under reduced pressure and the residue was purified column chromatography (SiO₂; MeOH/0.1% NH4_{O}H in CH₂Cl₂; gradient 0% to 10% MeOH) to give the title compound as a pale yellow oil (74.6 g, 119.4 mmol, 85.2% yield).

MS (ES⁺) C₃₄H₅₁N₅O₆ requires: 625, found: 626. [M+H]⁺.

¹H NMR (400 MHz, CDCl3) δ 7.37-7.27 (m, 5H), 5.56 (s, 1H), 5.18 (s, 2H), 4.63 (s, 2H), 4.14-4.09 (m, 2H), 3.94-3.92 (m, 2H), 3.46 (s, 2H), 3.35-3.32 (m, 8H), 3.30-3.25 (m, 2H), 2.81 (s, 3H), 2.51-2.50 (m, 2H), 2.25 (s, 3H), 2.18 (s, 2H), 2.02 (m, 2H), 1.80 (bs, 2H), 1.64-1.60 (m, 2H), 1.41 (s, 9H).

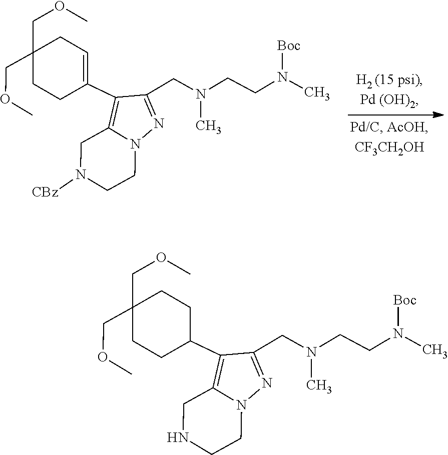

Step 5: tert-Butyl (2-(((3-(4,4-bis(methoxymethyl)cyclohexyl)-4,5,6,7-tetrahydropyrazolo[1,5-a]pyrazin-2-yl)methyl)(methyl)amino)ethyl)(methyl)carbamate: A Parr vessel was charged with the product from the previous step (89 g, 142.2 mmol), 2, 2, 2-trifluoroethanol (1000 mL) and AcOH (18.7 g, 17.8 mL, 311.2 mmol). The flask was purged with N₂, and Pd/C (8 g, 10% w/w) and Pd(OH)₂/C (8 g, 20% w/w) were added under a N₂ atmosphere. The vessel was set up on the Parr shaker, purged again with N₂ (3×), then purged with H₂ (5×) and shaken under an atmosphere of H₂ at 15 psi at 25° C. for 12 hours. The H₂ atmosphere was evacuated, the vessel was back-filled with N₂, additionally purged with N₂ (3×) and the mixture was filtered through a pad of CELITE®. The filtrate was placed again in a Parr vessel under N₂ atmosphere, and another portion of Pd/C (6 g, 10% w/w) and Pd (OH)₂/C (6 g, 20% w/w) were added to the solution. The vessel was set up on the Parr shaker, purged with N₂ (3×), purged with H₂ (5×) and shaken under an atmosphere of H₂ at 15 psi at 25° C. for an additional 6 hours. As described above, the H₂ atmosphere was removed and back-filled with N₂ (3×), and the mixture was filtered through a pad of CELITE®. The filtrate was concentrated under reduced pressure, the residue was diluted with dichloromethane (500 mL) and washed with saturated NaHCO₃ (100 mL). The organic layer was dried over Na₂SO₄ and concentrated under reduced pressure to give the title compound as an oil (69 g) which was used without further purification.

MS (ES⁺) C₂₆H₄₇N₅O₄ requires:493, found: 494. [M+H]⁺.

¹H NMR (400 MHz, CDCl3) δ 4.08-4.03 (m, 4H), 3.45 (s, 2H), 3.39 (s, 2H), 3.35 (s, 3H), 3.32 (s, 3H), 3.27-3.22 (m, 3H), 3.14 (s, 2H), 2.81 (s, 3H), 2.52-2.49 (m, 3H), 2.23 (s, 3H), 2.15-2.02 (m, 1H), 1.71-1.56 (m, 6H), 1.41 (s, 9H), 1.28-1.23 (m, 2H).

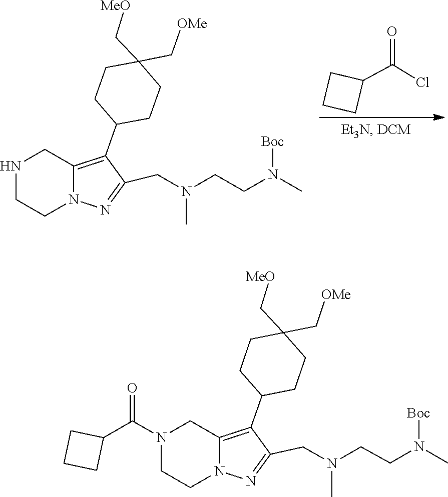 [SMILES: COCC1(COC)CCC(c2c(CN(C)CCN(C)C(=O)OC(C)(C)C)nn3c2CNCC3)CC1 ; O=C(Cl)C1CCC1 ; COCC1(COC)CCC(c2c(CN(C)CCN(C)C(=O)OC(C)(C)C)nn3c2CN(C(=O)C2CCC2)CC3)CC1]

Step 6: tert-Butyl (2-(((3-(4,4-bis(methoxymethyl)cyclohexyl)-5-(cyclobutanecarbonyl)-4,5,6,7-tetrahydropyrazolo[1,5-a]pyrazin-2-yl)methyl)(methyl)-amino)ethyl)(methyl)carbamate: To a solution of the product from the previous step (21.8 g, 44.2 mmol) in CH₂Cl₂(200 mL) at 0° C. was added Et₃N (13.4 g, 18.44 mL, 132.5 mmol), followed by dropwise addition of cyclobutanecarbonyl chloride (6.28 g, 6.04 mL, 53.0 mmol). The mixture was stirred at 20° C. for 1 hour, then concentrated under reduced pressure. The residue was triturated with EtOAc (100 mL), and the resulting suspension was filtered and the filtrate was concentrated under reduced pressure. The residue was purified column chromatography (SiO₂; MeOH/0.1% NH₄OH in CH₂Cl₂, gradient 0% to 8% MeOH) to give the title compound as a colorless oil (18.5 g, 32.3 mmol, 73% yield).

MS (ES⁺) C₃₁H₅₃N₅O₅ requires: 575, found: 576. [M+H]⁺.

¹H NMR (400 MHz, CDCl3) δ 4.76 (s, 1H), 4.53 (s, 1H), 4.10 (m, 1H), 4.07 (m, 1H), 3.73 (m, 1H), 3.49-3.39 (m, 4H), 3.36 (s, 3H), 3.34-3.31 (m, 4H), 3.28-3.27 (m, 2H), 3.18-3.10 (m, 2H), 2.80-2.79 (m, 3H), 2.49 (m, 3H), 2.40-2.31 (m, 2H), 2.27-2.09 (m, 6H), 1.97 (m, 1H), 1.85 (m, 1H), 1.77-1.49 (m, 6H), 1.40 (s, 9H), 1.30-1.15 (m, 2H).

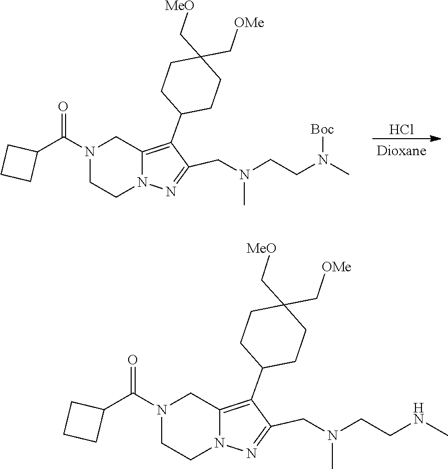 [SMILES: COCC1(COC)CCC(c2c(CN(C)CCN(C)C(=O)OC(C)(C)C)nn3c2CN(C(=O)C2CCC2)CC3)CC1 ; Cl ; CNCCN(C)Cc1nn2c(c1C1CCC(COC)(COC)CC1)CN(C(=O)C1CCC1)CC2]

Step 7: (3-(4,4-Bis(methoxymethyl)cyclohexyl)-2-((methyl(2-(methylamino)-ethyl)amino)methyl)-6,7-dihydropyrazolo[1,5-a]pyrazin-5(4H)-yl)(cyclobutyl)methanone: To a solution of the product from the previous step (20 g, 34.74 mmol) in dioxane (180 mL) at 0° C. was added freshly prepared HCl in dioxane (4M, 30 mL, 120 mmol). The mixture was stirred at 20° C. for 1 hour, then concentrated under reduced pressure without heating, and the residue was lyophilized from deionized water to give the title compound as white solid (bis HCl salt; 18.86 g, 33.7 mmol, 96.9% yield). The title compound was also obtained as a free base upon partitioning between CH₂Cl₂ and saturated aq. NaHCO₃, drying the organic phase over Na₂SO₄, and concentrating it under reduced pressure; MS (ES⁺) C₂₆H₄₅N₅O₃ requires: 475, found: 476. [M+H]⁺.

Hydrochloride salt: ¹H NMR (400 MHz, D₂O) δ 4.84 (s, 2H), 4.40 (s, 2H), 4.22-4.10 (m, 2H), 3.99-3.98 (m, 1H), 3.89-3.88 (m, 1H), 3.75 (s, 2H), 3.64-3.46 (m, 5H), 3.40-3.34 (m, 3H), 3.31 (s, 3H), 3.26-3.16 (m, 2H), 2.87 (s, 3H), 2.77 (s, 3H), 2.56-2.44 (m, 1H), 2.27-2.10 (m, 4H), 2.04-1.90 (m, 1H), 1.85-1.74 (m, 1H), 1.70-1.55 (m, 6H), 1.45-1.36 (m, 2H).

Hydrochloride salt: ¹H NMR (400 MHz, DMSO-d₆) δ 10.85 (bs, 1H), 9.32 (bs, 2H), 4.78-4.65 (m, 2H), 4.33 (bs, 2H), 4.16-4.07 (m, 2H), 3.93 (m, 1H), 3.81 (m, 1H), 3.57 (s, 3H), 3.50 (m, 1H), 3.41-3.30 (m, 6H), 3.24 (s, 3H), 3.10 (bs, 2H), 2.80 (s, 3H), 2.61-2.54 (m, 4H), 2.25-2.19 (m, 2H), 2.18-2.11 (m, 2H), 1.94 (m, 1H), 1.77 (m, 1H), 1.62-1.55 (m, 6H), 1.40-1.36 (m, 2H).

Free base: ¹H NMR (400 MHz, DMSO-d₆) δ 4.68-4.57 (m, 2H), 4.03-3.78 (m, 3H), 3.75 (m, 1H), 3.48 (m, 1H), 3.40-3.34 (m, 6H), 3.24-3.15 (m, 5H), 3.17 (s, 1H), 3.08 (s, 2H), 2.57-2.49 (m, 2H), 2.41-2.37 (m, 2H), 2.27 (s, 3H), 2.24-2.12 (m, 4H), 2.08 (s, 3H), 1.92 (m, 1H), 1.78 (m, 1H), 1.67-1.58 (m, 4H), 1.50-1.45 (m, 2H), 1.30-1.20 (m, 2H).

### EXAMPLE 2

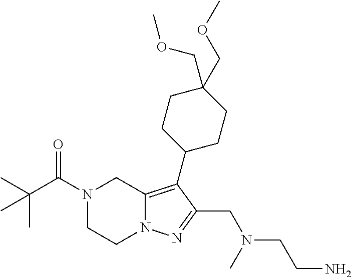 [SMILES: COCC1(COC)CCC(c2c(CN(C)CCN)nn3c2CN(C(=O)C(C)(C)C)CC3)CC1]

### 1-(2-(((2-aminoethyl)(methyl)amino)methyl)-3-(4,4-bis(methoxymethyl)cyclohexyl)-6,7-dihydropyrazolo[1,5-a]pyrazin-5(4H)-yl)-2,2-dimethylpropan-1-one

The title compound was prepared as described for Example 1, using tert-butyl (2-(methylamino)ethyl)carbamate instead of tert-butyl N-methyl-N-[2-(methylamino)ethyl]-carbamate in Step 3, and 2,2-dimethylpropanoyl chloride instead of cyclobutanecarbonyl chloride in Step 6. After Step 7, the compound was purified by prep-HPLC (Mobile phase: A=H₂O/0.05% HCl; B=MeCN; Gradient: B 1%-30% in 20 minutes; Column: Phenomenex Luna C18 10 μm, 250 mm×50 mm).

MS(ES⁺) C₂₅H₄₅N₅O₃ requires: 463, found 464 [M+H]⁺.

¹H NMR (400 MHz, D₂O) δ 4.95 (s, 2H), 4.39 (s, 2H), 4.25-4.06 (m, 4H), 3.60-3.41 (m, 6H), 3.38-3.27 (m, 6H), 3.22 (s, 2H), 2.87 (s, 3H), 2.51 (m, 1H), 1.69-1.52 (m, 6H), 1.29-1.26 (in, 11H).

The following compounds were prepared by the methods as set forth above.

TABLE 1
Example compounds 3-81.

| | | | MW/ |
|---|---|---|---|
| Ex | Structure | IUPAC Name | M + H |
| 3 | 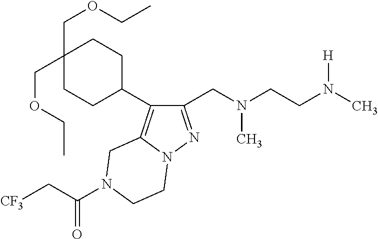 [SMILES: CCOCC1(COCC)CCC(c2c(CN(C)CCNC)nn3c2CN(C(=O)CC(F)(F)F)CC3)CC1] | 1-(3-(4,4-bis(ethoxymethyl)- cyclohexyl)-2-((methyl(2- (methylamino)ethyl)amino)- methyl)-6,7-dihydropyrazolo- [1,5-a]pyrazin-5(4H)-yl)- 3,3,3-trifluoropropan-1-one | 531/ 532 |
| 4 | 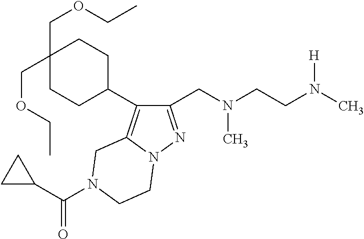 [SMILES: CCOCC1(COCC)CCC(c2c(CN(C)CCNC)nn3c2CN(C(=O)C2CC2)CC3)CC1] | (3-(4,4-bis(ethoxymethyl)- cyclohexyl)-2-((methyl(2- (methylamino)ethyl)amino)- methyl)-6,7-dihydropyrazolo- [1,5-a]pyrazin-5(4H)-yl)- (cyclopropyl)methanone | 489/ 490 |
| 5 | 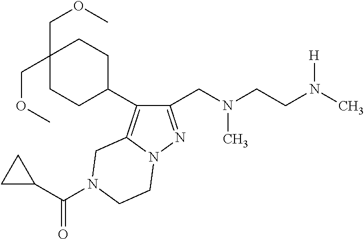 [SMILES: CNCCN(C)Cc1nn2c(c1C1CCC(COC)(COC)CC1)CN(C(=O)C1CC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (cyclopropyl)methanone | 461/ 462 |
| 6 | 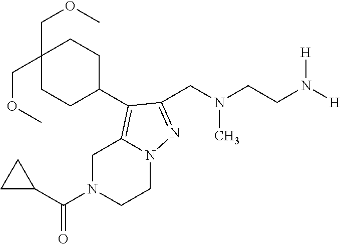 [SMILES: COCC1(COC)CCC(c2c(CN(C)CCN)nn3c2CN(C(=O)C2CC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)- (cyclopropyl)methanone | 447/ 448 |
| 7 | 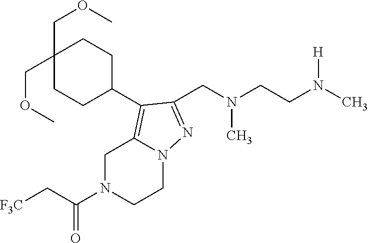 [SMILES: CNCCN(C)Cc1nn2c(c1C1CCC(COC)(COC)CC1)CN(C(=O)CC(F)(F)F)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-3,3,3- trifluoropropan-1-one | 503/ 504 |
| 8 | 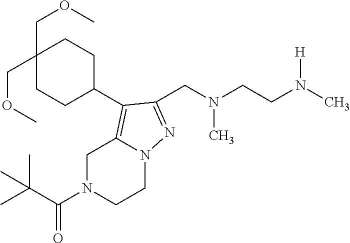 [SMILES: CNCCN(C)Cc1nn2c(c1C1CCC(COC)(COC)CC1)CN(C(=O)C(C)(C)C)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2,2- dimethylpropan-1-one | 477/ 478 |
| 9 | 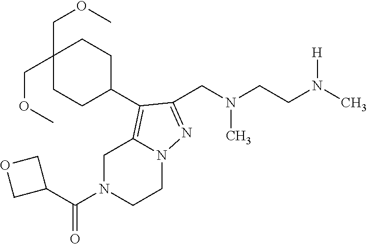 [SMILES: CNCCN(C)Cc1nn2c(c1C1CCC(COC)(COC)CC1)CN(C(=O)C1COC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (oxetan-3-yl)methanone | 477/ 478 |
| 10 | 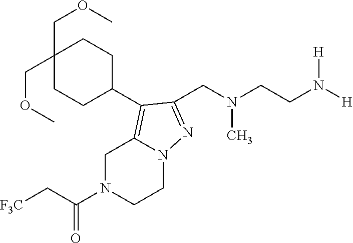 [SMILES: COCC1(COC)CCC(c2c(CN(C)CCN)nn3c2CN(C(=O)CC(F)(F)F)CC3)CC1] | 1-(2-(((2-aminoethyl)- (methyl)amino)methyl)-3- (4,4-bis(methoxymethyl)- cyclohexyl)-6,7-dihydro- pyrazolo[1,5-a]pyrazin- 5(4H)-yl)-3,3,3- trifluoropropan-1-one | 489/ 490 |
| 11 | 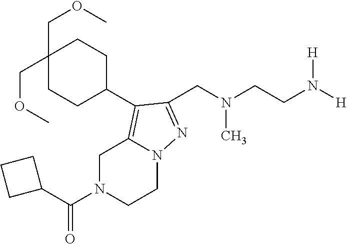 [SMILES: COCC1(COC)CCC(c2c(CN(C)CCN)nn3c2CN(C(=O)C2CCC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)- (cyclobutyl)methanone | 461/ 462 |
| 12 | 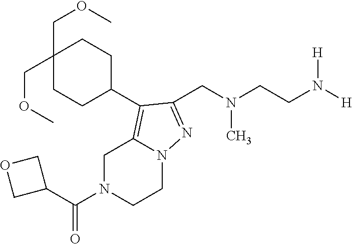 [SMILES: COCC1(COC)CCC(c2c(CN(C)CCN)nn3c2CN(C(=O)C2COC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(oxetan-3- yl)methanone | 463/ 464 |
| 13 | 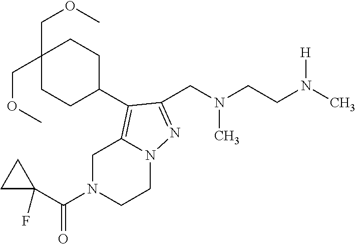 [SMILES: CNCCN(C)Cc1nn2c(c1C1CCC(COC)(COC)CC1)CN(C(=O)C1(F)CC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- fluorocyclopropyl)methanone | 479/ 480 |
| 14 | 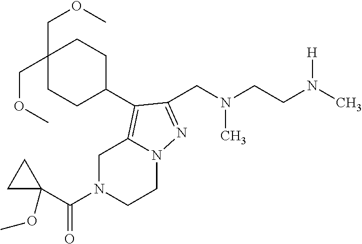 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- methoxycyclopropyl)- methanone | 491/ 492 |
| 15 | 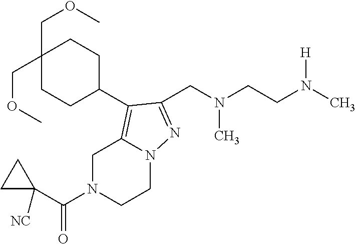 [SMILES: CNCCN(C)Cc1nn2c(c1C1CCC(COC)(COC)CC1)CN(C(=O)C1(C#N)CC1)CC2] | 1-(3-(4,4-bis(methoxy- methyl)cyclohexyl)-2- ((methyl(2-(methylamino)- ethyl)amino)methyl)-4,5,6,7- tetrahydropyrazolo[1,5-a]- pyrazine-5- carbonyl)cyclopropane-1- carbonitrile | 486/ 487 |
| 16 | 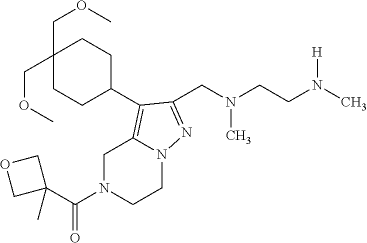 [SMILES: CNCCN(C)Cc1nn2c(c1C1CCC(COC)(COC)CC1)CN(C(=O)C1(C)COC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- methyloxetan-3-yl)methanone | 491/ 492 |
| 17 | 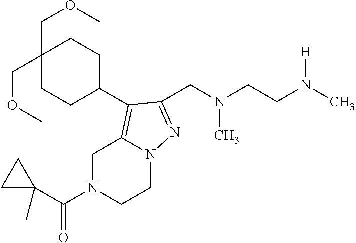 [SMILES: CNCCN(C)Cc1nn2c(c1C1CCC(COC)(COC)CC1)CN(C(=O)C1(C)CC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- methylcyclopropyl)- methanone | 475/ 476 |
| 18 | 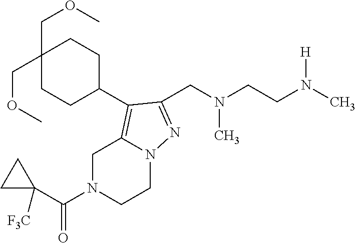 [SMILES: CNCCN(C)Cc1nn2c(c1C1CCC(COC)(COC)CC1)CN(C(=O)C1(C(F)(F)F)CC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- (trifluoro- methyl)cyclopropyl)- methanone | 529/ 530 |
| 19 | 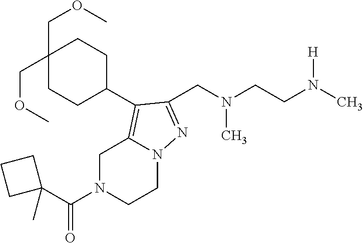 [SMILES: CNCCN(C)Cc1nn2c(c1C1CCC(COC)(COC)CC1)CN(C(=O)C1(C)CCC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- methylcyclobutyl)methanone | 489/ 490 |
| 20 | 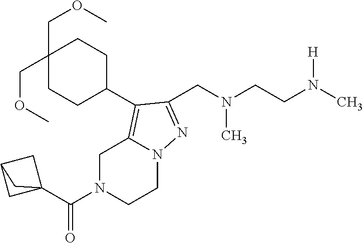 [SMILES: CNCCN(C)Cc1nn2c(c1C1CCC(COC)(COC)CC1)CN(C(=O)C13CC(C1)C3)CC2] | bicyclo[1.1.1]pentan-1-yl(3- (4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- methanone | 487/ 488 |
| 21 | 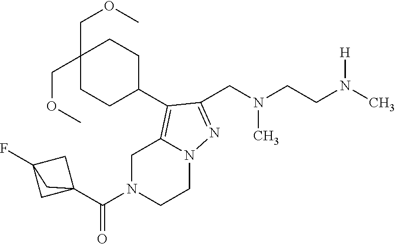 [SMILES: CNCCN(C)Cc1nn2c(c1C1CCC(COC)(COC)CC1)CN(C(=O)C13CC(F)(C1)C3)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- fluorobicyclo[1.1.1]pentan-1- yl)methanone | 505/ 506 |
| 22 | 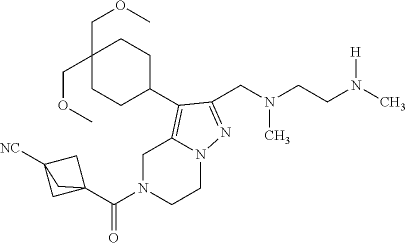 [SMILES: CNCCN(C)Cc1nn2c(c1C1CCC(COC)(COC)CC1)CN(C(=O)C13CC(C#N)(C1)C3)CC2] | 3-(3-(4,4-bis(methoxy- methyl)cyclohexyl)-2- ((methyl(2-(methylamino)- ethyl)amino)methyl)-4,5,6,7- tetrahydropyrazolo[1,5-a]- pyrazine-5- carbonyl)bicyclo[1.1.1] pentane-1-carbonitrile | 512/ 513 |
| 23 | 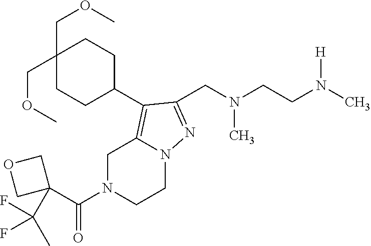 [SMILES: CNCCN(C)Cc1nn2c(c1C1CCC(COC)(COC)CC1)CN(C(=O)C1(C(C)(F)F)COC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- (1,1-difluoroethyl)oxetan-3- yl)methanone | 541/ 542 |
| 24 | 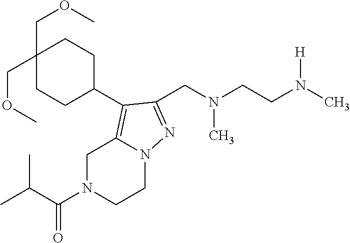 [SMILES: CNCCN(C)Cc1nn2c(c1C1CCC(COC)(COC)CC1)CN(C(=O)C(C)C)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- methylpropan-1-one | 463/ 464 |
| 25 | 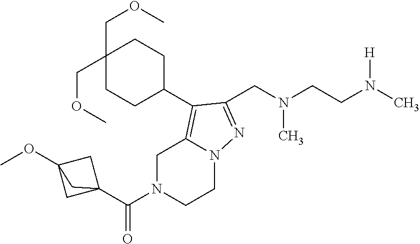 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- methoxybicyclo[1.1.1]pentan- 1-yl)methanone | 517/ 518 |
| 26 | 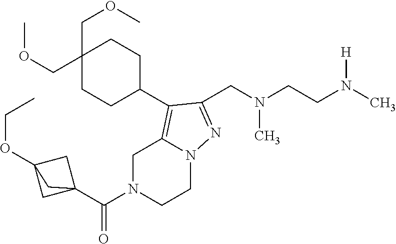 [SMILES: CCOC12CC(C(=O)N3CCn4nc(CN(C)CCNC)c(C5CCC(COC)(COC)CC5)c4C3)(C1)C2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- ethoxybicyclo[1.1.1]pentan- 1-yl)methanone | 505/ 506 |
| 27 | 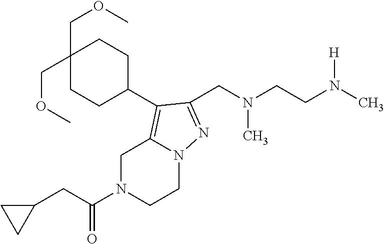 [SMILES: CNCCN(C)Cc1nn2c(c1C1CCC(COC)(COC)CC1)CN(C(=O)CC1CC1)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- cyclopropylethan-1-one | 475/ 476 |
| 28 | 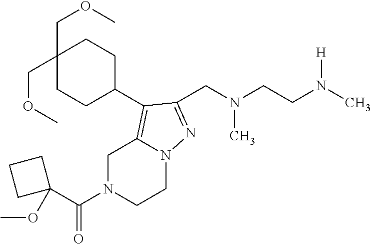 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- methoxycyclobutyl)- methanone | 505/ 506 |
| 29 | 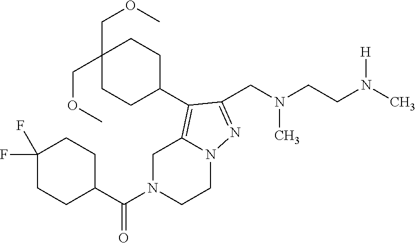 [SMILES: CNCCN(C)Cc1nn2c(c1C1CCC(COC)(COC)CC1)CN(C(=O)C1CCC(F)(F)CC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(4,4- difluorocyclohexyl)- methanone | 539/ 540 |
| 30 | 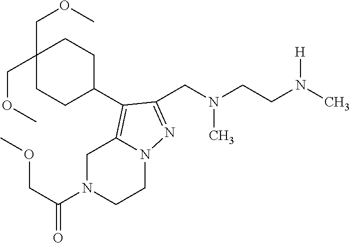 | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- methoxyethan-1-one | 465/ 466 |
| 31 | | | |
| | 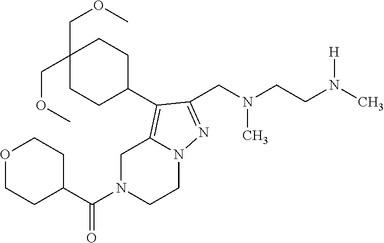 [SMILES: CNCCN(C)Cc1nn2c(c1C1CCC(COC)(COC)CC1)CN(C(=O)C1CCOCC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (tetrahydro-2H-pyran-4-yl)- methanone | 505/ 506 |
| 32 | 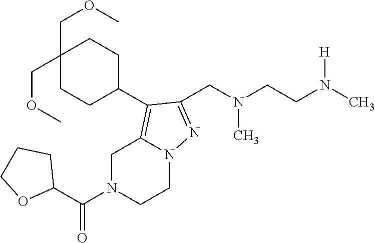 [SMILES: CNCCN(C)Cc1nn2c(c1C1CCC(COC)(COC)CC1)CN(C(=O)C1CCCO1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (tetrahydrofuran-2-yl)- methanone | 491/ 492 |
| 33 | 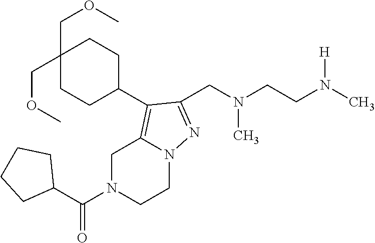 [SMILES: CNCCN(C)Cc1nn2c(c1C1CCC(COC)(COC)CC1)CN(C(=O)C1CCCC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (cyclopentyl)methanone | 489/ 490 |
| 34 | 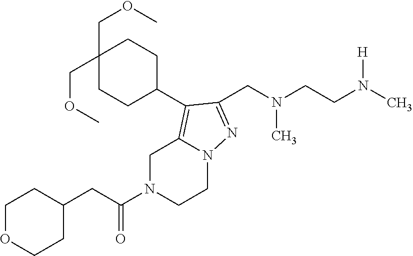 [SMILES: CNCCN(C)Cc1nn2c(c1C1CCC(COC)(COC)CC1)CN(C(=O)CC1CCOCC1)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- (tetrahydro-2H-pyran-4- yl)ethan-1-one | 519/ 520 |
| 35 | 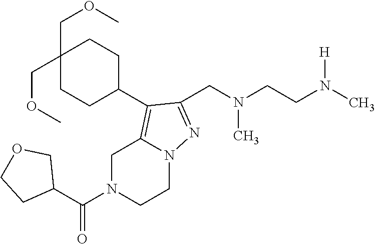 [SMILES: CNCCN(C)Cc1nn2c(c1C1CCC(COC)(COC)CC1)CN(C(=O)C1CCOC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (tetrahydrofuran-3-yl)- methanone | 491/ 492 |
| 36 | 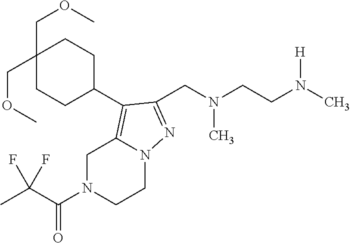 [SMILES: CNCCN(C)Cc1nn2c(c1C1CCC(COC)(COC)CC1)CN(C(=O)C(C)(F)F)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2,2- difluoropropan-1-one | 485/ 486 |
| 37 | 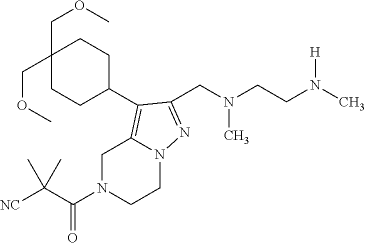 [SMILES: CNCCN(C)Cc1nn2c(c1C1CCC(COC)(COC)CC1)CN(C(=O)C(C)(C)C#N)CC2] | 3-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2,2- dimethyl-3-oxopropanenitrile | 488/ 489 |
| 38 | 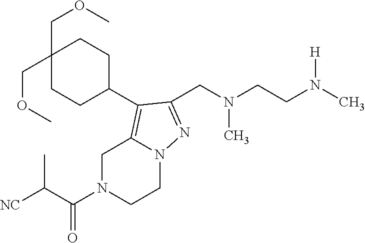 [SMILES: CNCCN(C)Cc1nn2c(c1C1CCC(COC)(COC)CC1)CN(C(=O)C(C)C#N)CC2] | 3-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2-methyl- 3-oxopropanenitrile | 474/ 475 |
| 39 | 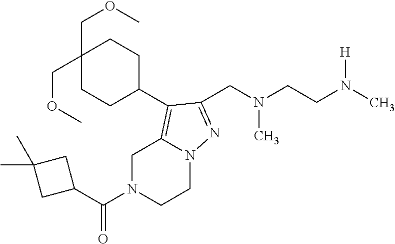 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3,3- dimethylcyclobutyl)- methanone | 503/ 504 |
| 40 | 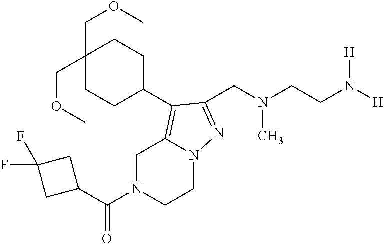 [SMILES: COCC1(COC)CCC(c2c(CN(C)CCN)nn3c2CN(C(=O)C2CC(F)(F)C2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(3,3- difluorocyclobutyl)- methanone | 497/ 498 |
| 41 | 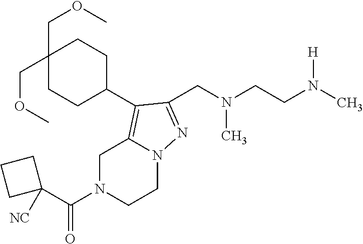 [SMILES: CNCCN(C)Cc1nn2c(c1C1CCC(COC)(COC)CC1)CN(C(=O)C1(C#N)CCC1)CC2] | 1-(3-(4,4-bis(methoxy- methyl)cyclohexyl)-2- ((methyl(2-(methylamino)- ethyl)amino)methyl)-4,5,6,7- tetrahydropyrazolo[1,5-a]- pyrazine-5- carbonyl)cyclobutane-1- carbonitrile | 500/ 501 |
| 42 | 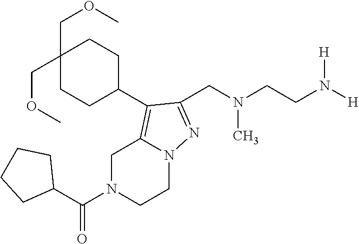 [SMILES: COCC1(COC)CCC(c2c(CN(C)CCN)nn3c2CN(C(=O)C2CCCC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)- (cyclopentyl)methanone | 475/ 476 |
| 43 | 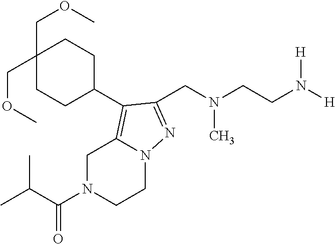 [SMILES: COCC1(COC)CCC(c2c(CN(C)CCN)nn3c2CN(C(=O)C(C)C)CC3)CC1] | 1-(2-(((2-aminoethyl)- (methyl)amino)methyl)-3- (4,4-bis(methoxymethyl)- cyclohexyl)-6,7-dihydro- pyrazolo[1,5-a]pyrazin- 5(4H)-yl)-2-methylpropan-1- one | 449/ 450 |
| 44 | 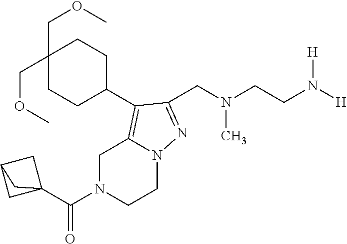 [SMILES: COCC1(COC)CCC(c2c(CN(C)CCN)nn3c2CN(C(=O)C24CC(C2)C4)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(bicyclo- [1.1.1]pentan-1-yl)methanone | 473/ 474 |
| 45 | 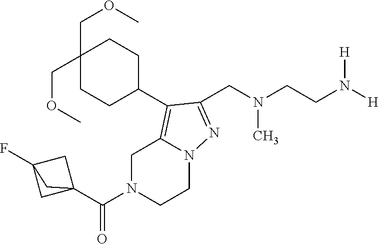 [SMILES: COCC1(COC)CCC(c2c(CN(C)CCN)nn3c2CN(C(=O)C24CC(F)(C2)C4)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(3- fluorobicyclo[1.1.1]pentan-1- yl)methanone | 491/ 492 |
| 46 | 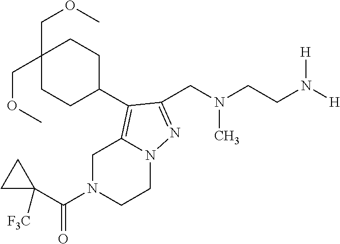 [SMILES: COCC1(COC)CCC(c2c(CN(C)CCN)nn3c2CN(C(=O)C2(C(F)(F)F)CC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(1- (trifluoro- methyl)cyclopropyl)- methanone | 515/ 516 |
| 47 | 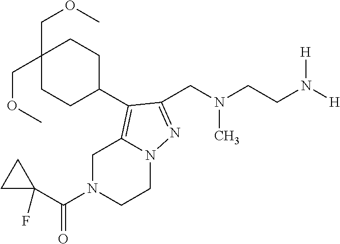 [SMILES: COCC1(COC)CCC(c2c(CN(C)CCN)nn3c2CN(C(=O)C2(F)CC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(1- fluorocyclopropyl)methanone | 465/ 466 |
| 48 | 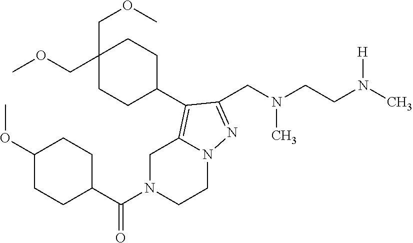 [SMILES: CNCCN(C)Cc1nn2c(c1C1CCC(COC)(COC)CC1)CN(C(=O)C1CCC(OC)CC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(4- methoxycyclohexyl)- methanone | 533/ 534 |
| 49 | 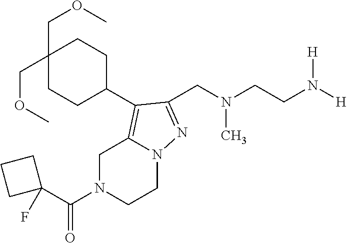 [SMILES: COCC1(COC)CCC(c2c(CN(C)CCN)nn3c2CN(C(=O)C2(F)CCC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(1-fluoro- cyclobutyl)methanone | 479/ 480 |
| 50 | 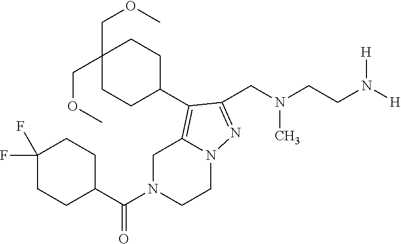 [SMILES: COCC1(COC)CCC(c2c(CN(C)CCN)nn3c2CN(C(=O)C2CCC(F)(F)CC2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(4,4- difluorocyclohexyl)- methanone | 525/ 526 |
| 51 | 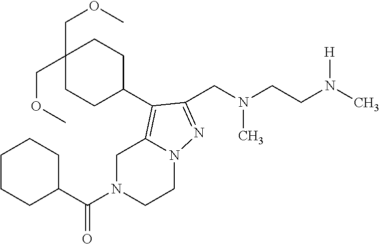 [SMILES: CNCCN(C)Cc1nn2c(c1C1CCC(COC)(COC)CC1)CN(C(=O)C1CCCCC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)- (cyclohexyl)methanone | 503/ 504 |
| 52 | 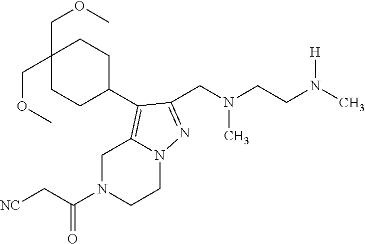 [SMILES: CNCCN(C)Cc1nn2c(c1C1CCC(COC)(COC)CC1)CN(C(=O)CC#N)CC2] | 3-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-3- oxopropanenitrile | 460/ 461 |
| 53 | 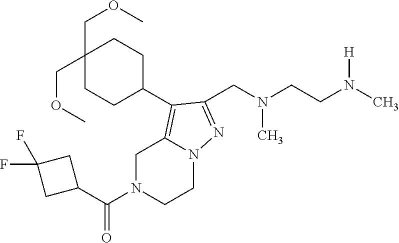 [SMILES: CNCCN(C)Cc1nn2c(c1C1CCC(COC)(COC)CC1)CN(C(=O)C1CC(F)(F)C1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3,3- difluorocyclobutyl)- methanone | 511/ 512 |
| 54 | 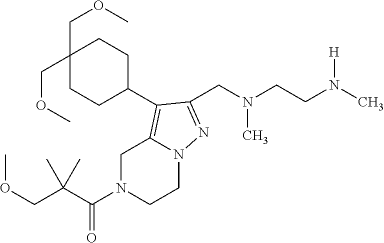 [SMILES: CNCCN(C)Cc1nn2c(c1C1CCC(COC)(COC)CC1)CN(C(=O)C(C)(C)COC)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-3- methoxy-2,2-dimethylpropan- 1-one | 507/ 508 |
| 55 | 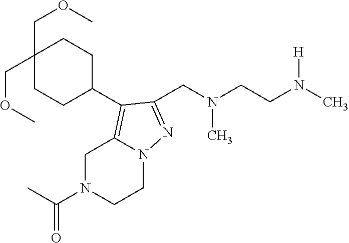 [SMILES: CNCCN(C)Cc1nn2c(c1C1CCC(COC)(COC)CC1)CN(C(C)=O)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)ethan-1-one | 435/ 436 |
| 56 | 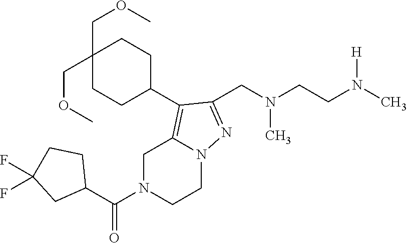 [SMILES: CNCCN(C)Cc1nn2c(c1C1CCC(COC)(COC)CC1)CN(C(=O)C1CCC(F)(F)C1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3,3- difluorocyclopentyl)- methanone | 525/ 526 |
| 57 | 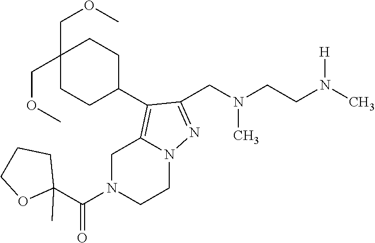 [SMILES: CNCCN(C)Cc1nn2c(c1C1CCC(COC)(COC)CC1)CN(C(=O)C1(C)CCCO1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(2- methyltetrahydrofuran-2-yl)- methanone | 505/ 506 |
| 58 | 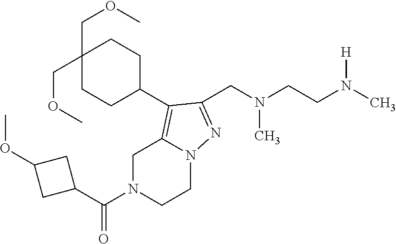 [SMILES: CNCCN(C)Cc1nn2c(c1C1CCC(COC)(COC)CC1)CN(C(=O)C1CC(OC)C1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- methoxycyclobutyl)- methanone | 505/ 506 |
| 59 | 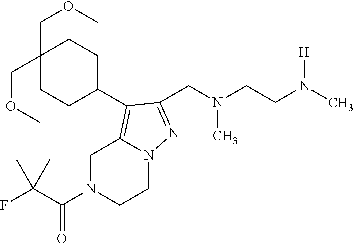 [SMILES: CNCCN(C)Cc1nn2c(c1C1CCC(COC)(COC)CC1)CN(C(=O)C(C)(C)F)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2-fluoro-2- methylpropan-1-one | 481/ 482 |
| 60 | 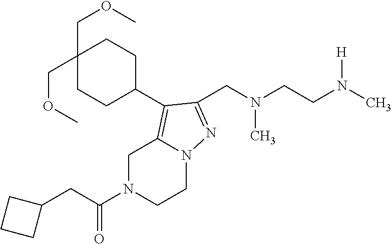 [SMILES: CNCCN(C)Cc1nn2c(c1C1CCC(COC)(COC)CC1)CN(C(=O)CC1CCC1)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- cyclobutylethan-1-one | 489/ 490 |
| 61 | 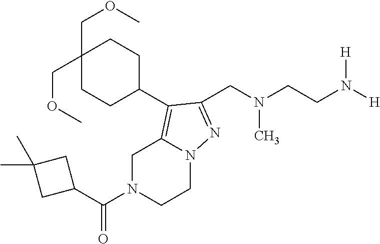 [SMILES: COCC1(COC)CCC(c2c(CN(C)CCN)nn3c2CN(C(=O)C2CC(C)(C)C2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(3,3- dimethylcyclobutyl)- methanone | 489/ 490 |
| 62 | 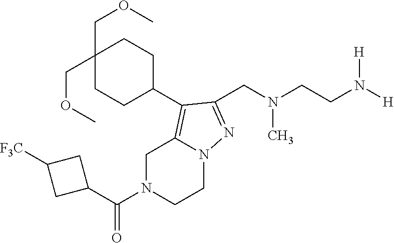 [SMILES: COCC1(COC)CCC(c2c(CN(C)CCN)nn3c2CN(C(=O)C2CC(C(F)(F)F)C2)CC3)CC1] | (2-(((2-aminoethyl)(methyl)- amino)methyl)-3-(4,4-bis- (methoxymethyl)cyclohexyl)- 6,7-dihydropyrazolo[1,5-a]- pyrazin-5(4H)-yl)(3- (trifluoromethyl)cyclobutyl)- methanone | 529/ 530 |
| 63 | 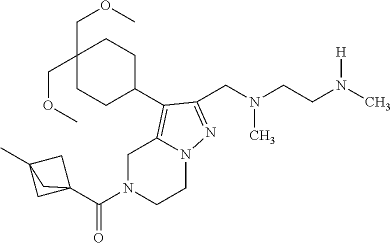 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- methylbicyclo[1.1.1]pentan- 1-yl)methanone | 501/ 502 |
| 64 | 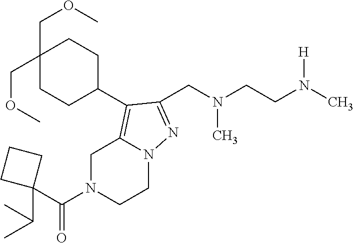 [SMILES: CNCCN(C)Cc1nn2c(c1C1CCC(COC)(COC)CC1)CN(C(=O)C1(C(C)C)CCC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- isopropylcyclobutyl)- methanone | 517/ 518 |
| 65 | 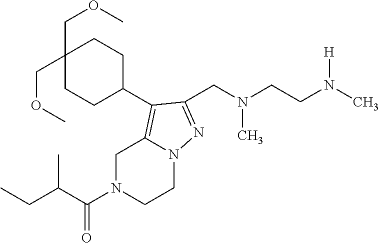 [SMILES: CCC(C)C(=O)N1CCn2nc(CN(C)CCNC)c(C3CCC(COC)(COC)CC3)c2C1] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- methylbutan-1-one | 477/ 478 |
| 66 | 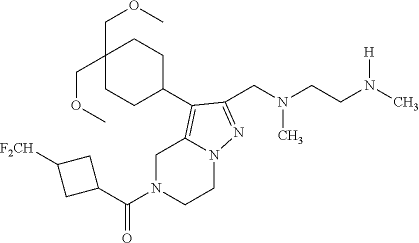 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- (difluoromethyl)cyclobutyl)- methanone | 541/ 542 |
| 67 | 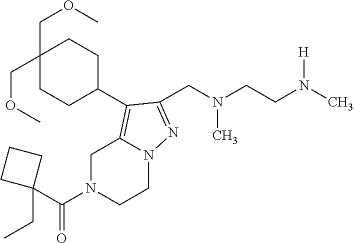 [SMILES: CCC1(C(=O)N2CCn3nc(CN(C)CCNC)c(C4CCC(COC)(COC)CC4)c3C2)CCC1] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- ethylcyclobutyl)methanone | 503/ 504 |
| 68 | 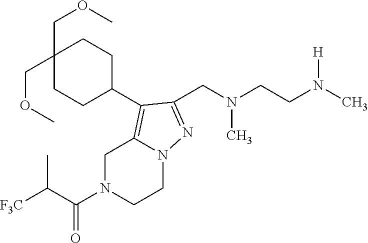 [SMILES: CNCCN(C)Cc1nn2c(c1C1CCC(COC)(COC)CC1)CN(C(=O)C(C)C(F)(F)F)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-3,3,3- trifluoro-2-methylpropan-1- one | 517/ 518 |
| 69 | 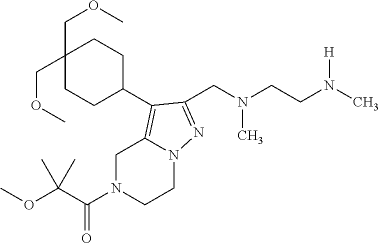 | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- methoxy-2-methylpropan-1- one | 493/ 494 |
| 70 | 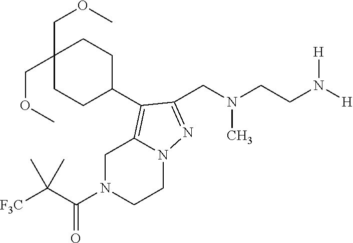 [SMILES: COCC1(COC)CCC(c2c(CN(C)CCN)nn3c2CN(C(=O)C(C)(C)C(F)(F)F)CC3)CC1] | 1-(2-(((2-aminoethyl)- (methyl)amino)methyl)-3- (4,4-bis(methoxymethyl)- cyclohexyl)-6,7-dihydro- pyrazolo[1,5-a]pyrazin- 5(4H)-yl)-3,3,3-trifluoro-2,2- dimethylpropan-1-one | 517/ 518 |
| 71 | 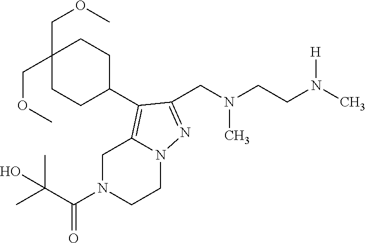 [SMILES: CNCCN(C)Cc1nn2c(c1C1CCC(COC)(COC)CC1)CN(C(=O)C(C)(C)O)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2-hydroxy- 2-methylpropan-1-one | 479/ 480 |
| 72 | 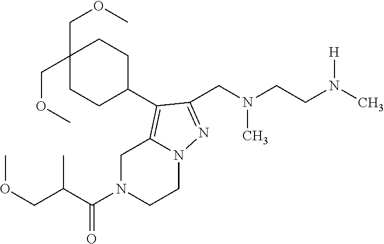 [SMILES: CNCCN(C)Cc1nn2c(c1C1CCC(COC)(COC)CC1)CN(C(=O)C(C)COC)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-3- methoxy-2-methylpropan-1- one | 493/ 494 |
| 73 | 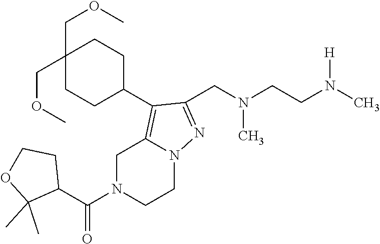 [SMILES: CNCCN(C)Cc1nn2c(c1C1CCC(COC)(COC)CC1)CN(C(=O)C1CCOC1(C)C)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(2,2- dimethyltetrahydrofuran-3- yl)methanone | 519/ 520 |
| 74 | 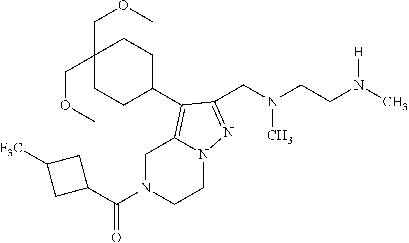 [SMILES: CNCCN(C)Cc1nn2c(c1C1CCC(COC)(COC)CC1)CN(C(=O)C1CC(C(F)(F)F)C1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- (trifluoromethyl)cyclobutyl)- methanone | 543/ 544 |
| 75 | 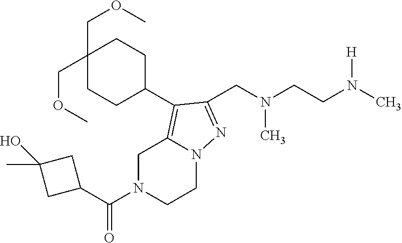 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- hydroxy-3- methylcyclobutyl)methanone | 505/ 506 |
| 76 | 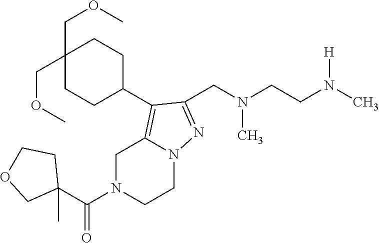 [SMILES: CNCCN(C)Cc1nn2c(c1C1CCC(COC)(COC)CC1)CN(C(=O)C1(C)CCOC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3- methyltetrahydrofuran-3-yl)- methanone | 505/ 506 |
| 77 | 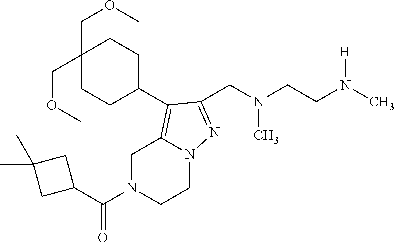 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(3,3- dimethylcyclobutyl)- methanone | 503/ 504 |
| 78 | 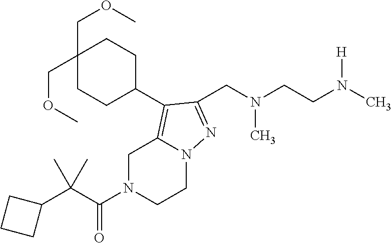 [SMILES: CNCCN(C)Cc1nn2c(c1C1CCC(COC)(COC)CC1)CN(C(=O)C(C)(C)C1CCC1)CC2] | 1-(3-(4,4-bis(methoxy- methyl)cyclohexyl)-2- ((methyl(2-(methylamino)- ethyl)amino)methyl)-6,7- dihydropyrazolo[1,5-a]- pyrazin-5(4H)-yl)-2- cyclobutyl-2-methylpropan-1- one | 517/ 518 |
| 79 | 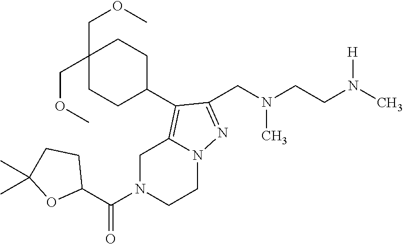 | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(5,5- dimethyltetrahydrofuran-2- yl)methanone | 519/ 520 |
| 80 | 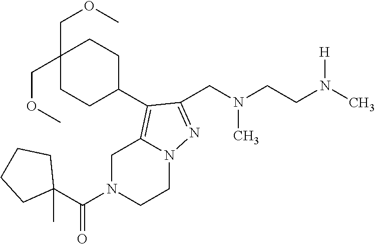 [SMILES: CNCCN(C)Cc1nn2c(c1C1CCC(COC)(COC)CC1)CN(C(=O)C1(C)CCCC1)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(1- methylcyclopentyl)- methanone | 503/ 504 |
| 81 | 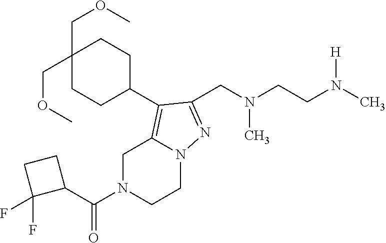 [SMILES: CNCCN(C)Cc1nn2c(c1C1CCC(COC)(COC)CC1)CN(C(=O)C1CCC1(F)F)CC2] | (3-(4,4-bis(methoxymethyl)- cyclohexyl)-2-((methyl(2- (methylamino)ethyl)amino)- methyl)-6,7-dihydropyrazolo- [1,5-a]pyrazin-5(4H)-yl)(2,2- difluorocyclobutyl)- methanone | 511/ 512 |

Certain compounds disclosed herein exist as diastereomers. This disclosure contemplates the use of individual purified diastereomers as well as mixtures. The mixtures can contain an essentially equal fractions of each possible diastereomer, or the mixtures can contain nonequal fractions of diastereomers, either as afforded directly from the reaction that creates the stereocenter, or from a later purification or separation step. Both mixtures of diastereomers and purified diastereomers can be assayed for biological activity. The absolute stereochemistry of certain individual diastereomers can be assigned based on spectroscopic and crystallographic techniques known in the art. Certain individual diastereomers can be assigned based on physical properties, for example, retention time on a chromatographic column.

### Biological Activity Assay

### PRMT1 Enzymatic Assay

In order to measure PRMT1 enzymatic activity the LANCE TR-FRET assay from PerkinElmer was used to follow the methylation of histone H4 at Arg3 using S-adenosyl-L-methionine (SAM) as the methyl group donor.

This enzymatic assay was performed in a 384 well, white, low volume plate (PerkinElmer, Catalog 6008289) with assay buffer consisting of 50 mM Hepes (pH 8) (Teknova, Catalog #H1090), 1 mM TCEP (Sigma, Catalog #C4706), and 0.003% Tween-20 (Thermo, Catalog #85114). Stock solutions of the test compounds were prepared in 100% DMSO (Sigma, Catalog #D2650) and serially diluted 1:3 using 100% DMSO. Compounds were additionally diluted 1:40 in assay buffer, and 2 uL/well were transferred to the assay plate. 4 uL/well (final concentration 1.5 nM) of PRMT1 protein (SignalChem, Catalog #P365-380G) diluted in assay buffer was added to the assay plate followed by a 15 min preincubation at room temperature. 4 uL/well of SAM (Sigma, Catalog #A7007) and biotinylated histone H4 (1-21) (AnaSpec, Catalog #62555) (final concentrations 1 μM and 25 nM, respectively) diluted in assay buffer were then added to the assay plate followed by a 1 hour reaction time. Final concentrations of PRMT1, SAM, and histone H4 (1-21) refer to a 10 L volume.

Detection of methylated histone H4 (H4R³me) was achieved by combining LANCE Ultra Europium-anti-H4R³me antibody (PerkinElmer, Catalog #TRF04-14), LANCE Ultra ULight-anti-streptavidin antibody (PerkinElmer, Catalog #TRF0102), and sinefungin (Sigma, Catalog #S8559) (final concentrations 2 nM, 50 nM, and 100 μM respectively) in 1× LANCE detection buffer (PerkinElmer, Catalog #CR97-100) and adding L/well of the detection solution to the assay plate. Detection reagents were allowed to react for 1 hour at room temperature. Final concentrations in the detection solution refer to a L volume. The europium fluorescence signal and the ULight TR-FRET signal were measured using a BioTek Synergy Neo plate reader: excitation at 330 nm, emission at 620 nm and 665 nm respectively, and the ratio of the two signals (665 nm/620 nm) was used for curve fitting. IC₅₀ values were calculated using a four-parameter logistic curve fit using Genedata Screener software.

### PRMT1 RKO Cellular Target Engagement Assay

RKO cells were routinely maintained in EMEM media (ATCC, Catalog #30-2003) supplemented with 10% fetal bovine serum (Sigma, Catalog #F2442) using a humidified incubator (37° C., 5% CO₂, and ambient O₂).

In preparation for the In-Cell Western assay, cells were harvested and resuspended in EMEM media supplemented with 10% fetal bovine serum. Cells were seeded onto a 384 well, black, clear bottom, Poly-D-Lysine coated tissue culture plate (Greiner, Catalog #781946) at a density of 1,000 cells/well in a volume of 40 μL. The culture plate was incubated for 24 hr at 37° C. with 5% CO₂ and ambient O₂. Stock solutions of the test compounds were prepared in 100% DMSO (Sigma, Catalog #D2650) and serially diluted 1:3 using 100% DMSO. Compounds were additionally diluted 1:40 in culture medium, and 10 μL/well was transferred to the tissue culture plate. Following the compound addition the microplate was incubated at 37° C. for 48 hr.

The media was removed, the plate was washed with 1× PBS (Fisher Bioreagents, Catalog #BP399-20), and cells were fixed for 10 min using 30 μL/well of 4% paraformaldehyde (Electron Microscopy Sciences, Catalog #15710). The paraformaldehyde was removed, the plate was again washed with 1× PBS, and cells were permeabilized for 15 min using 30 μL/well of 1× PBS containing 0.5% Triton-X 100 (Sigma, Catalog #1001748095). The permeabilization buffer was removed, the plate was washed with 1× PBST (Boston BioProducts, Catalog #IBB-171X), and 50 μL/well of blocking buffer (LI-COR, Catalog #927-40000) was added followed by a 1 hour incubation at room temperature. The blocking buffer was removed, and 20 μL/well of anti-asymmetric di-methyl arginine antibody (Cell Signaling, Catalog #13522S) diluted 1:1000 in LI-COR blocking buffer was added to the plate and incubated overnight, in the dark at 4° C.

The primary antibody was then removed, and the plate was washed three times with 1× PBST. 20 μL/well of CellTag (LI-COR, Catalog #926-41090) and IRDye 800CW goat anti-rabbit IgG antibody (LI-COR, Catalog #926-32211), each diluted 1:500 in LI-COR blocking buffer supplemented with 0.1% Tween-20 (Thermo Scientific, Catalog #85114), were added to the plate. The plate was then incubated in the dark, at room temperature for 1 hour followed by three washes with 1× PBST and one wash with H₂O. The IRDye secondary antibody signal (800 channel) and the CellTag signal (700 channel) were measured using a Licor Odyssey Imager, and the 800 channel signal was then normalized to the 700 channel signal. IC₅₀ values were calculated using a four-parameter logistic curve fit using Genedata Screener software.

### PATC53 Long Term Phenotypic Assay

PATC53 cells were derived from the PATC53 PDX model and kindly provided by Jason Fleming (MDACC). The cells were routinely maintained in DMEM/F12 Medium (Corning, Catalog #10-090-CV) supplemented with 10% fetal bovine serum (Sigma, Catalog #F2442), 100 units/mL penicillin and 100 μg/mL streptomycin (Gibco, Catalog #15140-122) in a humidified incubator (37° C., 5% CO₂). Prior to the assay, cells were seeded onto a 12-well, TC treated plate (Corning, catalog #353043) at a density of 250 cells per well in a volume of 1 mL. The plate was incubated for 24 hours at 37° C. and 5% CO₂. Stock solutions of the test compounds were prepared in 100% DMSO (Sigma, Catalog #D2650) and serially diluted 1:3 using 100% DMSO. Compounds were additionally diluted 1:100 in culture medium, and 110 μL/well was transferred to the tissue culture plate. Following the compound addition, the plate was incubated at 37° C. and 5% CO₂ for 14 days. Viability was then assessed by crystal violet staining (Fisher Scientific, Catalog #C58125) as follows: 450 μL of crystal violet (1 mg/mL) was added to the plate for a 10 minute incubation at room temperature. After incubation, the plate was washed 3× with distilled water to remove excess dye and allowed to dry overnight. The crystal violet was solubilized using 10% HOAc (500 μL) for 10 minutes at room temperature, while shaking. A volume of 100 μL was then transferred to a 96-well SpectraPlate (Perkin Elmer, Catalog #6005649). Absorbance at 590 nm was measured with a BioTek Synergy Neo. IC₅₀ values were calculated using a four-parameter logistic curve fit using Genedata Screener software.

### DOHH2 Long Term Phenotypic Assay

DOHH2 cells were kindly provided by Michael Green (MDACC). The cells were routinely maintained in RPMI-1640 Medium (ATCC, Catalog #30-2001) supplemented with 10% fetal bovine serum (Sigma, Catalog #F2442), 100 units/mL penicillin and 100 μg/mL streptomycin (Gibco, Catalog #15140-122) in a humidified incubator (37° C., 5% CO₂). Cells were seeded onto a white, CulturPlate-384 (Perkin Elmer, catalog #6007680) at a density of 250 cells per well in a volume of 50 μL. Stock solutions of the test compounds were prepared in 100% DMSO (Sigma, Catalog #D2650) and serially diluted 1:3 using 100% DMSO. Compounds were additionally diluted 1:33 in culture medium, and 10 μL/well was transferred to the tissue culture plate. Following the compound addition, the plate was incubated at 37° C. and 5% CO₂ for 7 days. Cells were then mixed and a 30 μL volume was removed. Viability was assessed by a 30 μL addition of Cell Titer-Glo 2.0 (Promega, catalog #G9243). The plate was then shaken on an orbital shaker at 300 RPM for 10 minutes at room temperature, in the dark. Luminescence was measured using a PerkinElmer Envision™ plate reader. IC₅₀ values were calculated using a four-parameter logistic curve fit using Genedata Screener software.

TABLE 2
PRMT-1 Enzymatic
Assay IC₅₀ values

| Ex. | PRMT-1 |
|---|---|
| No | IC₅₀ (nM) |
| 1 | 35 |
| 2 | 16 |
| 3 | 57 |
| 4 | 48 |
| 5 | 34 |
| 6 | 14 |
| 7 | 32 |
| 8 | 36 |
| 9 | 65 |
| 10 | 20 |
| 11 | 16 |
| 12 | 23 |
| 13 | 56 |
| 14 | 94 |
| 15 | 71 |
| 16 | 64 |
| 17 | 56 |
| 18 | 60 |
| 19 | 52 |
| 20 | 62 |
| 21 | 53 |
| 22 | 72 |
| 23 | 137 |
| 24 | 76 |
| 25 | 82 |
| 26 | 439 |
| 27 | 59 |
| 28 | 139 |
| 29 | 65 |
| 30 | 81 |
| 31 | 108 |
| 32 | 46 |
| 33 | 55 |
| 34 | 124 |
| 35 | 62 |
| 36 | 50 |
| 37 | 90 |
| 38 | 103 |
| 39 | 79 |
| 40 | 54 |
| 41 | 72 |
| 42 | 29 |
| 43 | 27 |
| 44 | 17 |
| 45 | 18 |
| 46 | 29 |
| 47 | 15 |
| 48 | 156 |
| 49 | 13 |
| 50 | 17 |
| 51 | 58 |
| 52 | 71 |
| 53 | 68 |
| 54 | 103 |
| 55 | 70 |
| 56 | 54 |
| 57 | 80 |
| 58 | 65 |
| 59 | 48 |
| 60 | 54 |
| 61 | 23 |
| 62 | 23 |
| 63 | 44 |
| 64 | 112 |
| 65 | 70 |
| 66 | 52 |
| 67 | 75 |
| 68 | 121 |
| 69 | 320 |
| 70 | 44 |
| 71 | 266 |
| 72 | 112 |
| 73 | 118 |
| 74 | 57 |
| 75 | 70 |
| 76 | 86 |
| 77 | 84 |
| 78 | 68 |
| 79 | 217 |
| 80 | 91 |
| 81 | 133 |

TABLE 3
PRMT-1 Target
Engagement IC₅₀ values

| Ex. | RKO IC₅₀ |
|---|---|
| No | (nM) |
| 1 | 138 |
| 2 | 311 |
| 3 | 153 |
| 4 | 110 |
| 5 | 121 |
| 6 | 361 |
| 7 | 1624 |
| 8 | 913 |
| 9 | 1111 |
| 10 | 695 |
| 11 | 1214 |
| 12 | ND |
| 13 | 225 |
| 14 | 124 |
| 15 | 370 |
| 16 | 370 |
| 17 | 370 |
| 18 | 137 |
| 19 | 214 |
| 20 | 239 |
| 21 | 185 |
| 22 | 1403 |
| 23 | 382 |
| 24 | 254 |
| 25 | 419 |
| 26 | 2401 |
| 27 | 318 |
| 28 | 907 |
| 29 | 271 |
| 30 | 795 |
| 31 | ND |
| 32 | 293 |
| 33 | 120 |
| 34 | ND |
| 35 | 818 |
| 36 | 269 |
| 37 | 237 |
| 38 | 690 |
| 39 | 177 |
| 40 | 1515 |
| 41 | 180 |
| 42 | 431 |
| 43 | 460 |
| 44 | 210 |
| 45 | 830 |
| 46 | 486 |
| 47 | 671 |
| 48 | 1414 |
| 49 | 329 |
| 50 | 1237 |
| 51 | 10000 |
| 52 | 163 |
| 53 | 1111 |
| 54 | 203 |
| 55 | 1111 |
| 56 | 392 |
| 57 | 267 |
| 58 | 372 |
| 59 | 177 |
| 60 | 345 |
| 61 | 1948 |
| 62 | 1384 |
| 63 | 610 |
| 64 | 2326 |
| 65 | 534 |
| 66 | 958 |
| 67 | 273 |
| 68 | 128 |
| 69 | 430 |
| 70 | 260 |
| 71 | 3481 |
| 72 | 315 |
| 73 | 470 |
| 74 | 142 |
| 75 | 5650 |
| 76 | 363 |
| 77 | 254 |
| 78 | 192 |
| 79 | 688 |
| 80 | 116 |
| 81 | 367 |

TABLE 4
PATC53 IC50 values

| Ex. | PATC53 |
|---|---|
| No. | IC₅₀ (nM) |
| 1 | 45 |
| 2 | 152 |
| 3 | 428 |
| 4 | 1258 |
| 5 | 58 |
| 6 | 147 |
| 7 | 210 |
| 8 | 100 |
| 9 | 1305 |
| 10 | 649 |
| 11 | 70 |
| 12 | 3856 |
| 13 | 38 |
| 14 | 478 |
| 15 | 732 |
| 16 | 209 |
| 17 | 133 |
| 18 | 48 |
| 19 | 37 |
| 20 | 50 |
| 21 | 68 |
| 22 | 447 |
| 23 | 264 |
| 24 | 69 |
| 25 | 203 |
| 26 | ND |
| 27 | 115 |
| 28 | 435 |
| 29 | 84 |
| 30 | 795 |
| 31 | 595 |
| 32 | 163 |
| 33 | 55 |
| 34 | 642 |
| 35 | 655 |
| 36 | 246 |
| 37 | 171 |
| 38 | 580 |
| 39 | 49 |
| 40 | 304 |
| 41 | 175 |
| 42 | 136 |
| 43 | 149 |
| 44 | 123 |
| 45 | 178 |
| 46 | 294 |
| 47 | 45 |
| 48 | 247 |
| 49 | 118 |
| 50 | 154 |
| 51 | ND |
| 52 | ND |
| 53 | 42 |
| 54 | ND |
| 55 | ND |
| 56 | ND |
| 57 | ND |
| 58 | 131 |
| 59 | ND |
| 60 | 33 |
| 61 | 68 |
| 62 | ND |
| 63 | 16 |
| 64 | ND |
| 65 | ND |
| 66 | ND |
| 67 | ND |
| 68 | 41 |
| 69 | ND |
| 70 | ND |
| 71 | ND |
| 72 | ND |
| 73 | ND |
| 74 | 54 |
| 75 | ND |
| 76 | ND |
| 77 | ND |
| 78 | ND |
| 79 | ND |
| 80 | 70 |
| 81 | ND |

TABLE 5
DOHH2 IC₅₀ values

| Ex. | DOHH2 |
|---|---|
| No. | IC₅₀ (nM) |
| 1 | 66 |
| 2 | 158 |
| 3 | ND |
| 4 | 47 |
| 5 | 81 |
| 6 | 138 |
| 7 | ND |
| 8 | 67 |
| 9 | ND |
| 10 | ND |
| 11 | 111 |
| 12 | ND |
| 13 | 150 |
| 14 | 211 |
| 15 | 211 |
| 16 | 160 |
| 17 | ND |
| 18 | 142 |
| 19 | 87 |
| 20 | 103 |
| 21 | 96 |
| 22 | ND |
| 23 | 147 |
| 24 | 103 |
| 25 | 150 |
| 26 | 844 |
| 27 | 79 |
| 28 | 207 |
| 29 | 99 |
| 30 | 159 |
| 31 | 206 |
| 32 | 160 |
| 33 | 83 |
| 34 | 198 |
| 35 | 212 |
| 36 | 190 |
| 37 | 142 |
| 38 | 83 |
| 39 | 108 |
| 40 | 249 |
| 41 | 91 |
| 42 | 180 |
| 43 | 185 |
| 44 | 150 |
| 45 | 175 |
| 46 | 288 |
| 47 | 187 |
| 48 | 284 |
| 49 | 166 |
| 50 | 171 |
| 51 | 75 |
| 52 | 514 |
| 53 | 87 |
| 54 | 293 |
| 55 | 296 |
| 56 | 83 |
| 57 | 170 |
| 58 | 131 |
| 59 | 113 |
| 60 | 82 |
| 61 | 224 |
| 62 | 76 |
| 63 | 67 |
| 64 | 230 |
| 65 | 134 |
| 66 | 150 |
| 67 | 156 |
| 68 | 71 |
| 69 | 474 |
| 70 | 195 |
| 71 | 962 |
| 72 | 310 |
| 73 | 575 |
| 74 | 93 |
| 75 | 1444 |
| 76 | 94 |
| 77 | 202 |
| 78 | 154 |
| 79 | 303 |
| 80 | 104 |
| 81 | 296 |

### Anti-tumor Effects in Mouse Xenografts

The effect of the compounds of Examples 1 and 2 on tumor growth was assessed in a PANC-1 (pancreatic ductal adenocarcinoma) mouse xenograft model. Female C.B -Igh-I^{b}/IcrTac-Prkdc^{scid} (C.B-17 SCID) mice were injected subcutaneously in the right flank with a suspension of PANCG1 cells (5 million cells with 50% Matrigel (Corning #356231) in sterile Dulbecco's phosphate buffered saline (DPBS, Life Technologies #14190-144). After implantation, tumor volume (TV) was measured weekly and mice bearing tumors with volumes ˜200 mm³ were randomized into treatment groups of 8 mice each. Mice were dosed by oral gavage, twice daily for 28 days with either vehicle or 1 and 2 at the doses reported in Table 6 and Table 7 respectively. The doses were scaled to the body weights (BW) of individual animals at a dosing volume of 5 mL/Kg. Throughout the duration of study tumor growth was assessed by caliper measurement and treatment response was determined by percent tumor growth inhibition (% TGI; calculated as TGI %=(Ci−Ti)/(Ci−C₀)*100%; Ci and Ti are the mean tumor volumes of the vehicle and treatment groups on the measurement day, respectively; C₀ is the mean tumor volume of the vehicle group on day 0 (i.e. the day before treatment started). Mouse body weight was measured bi-weekly and reported as percentage of BW change from day 0 (calculated as Body Weight Change %=(BWi−BW₀)/BW₀*100%; BWi and BW₀ are body weight of an individual mouse on measurement day i and on day 0, respectively). Statistical significance was calculated by repeated-measures two-way ANOVA with multiple comparisons and Tukey correction using GraphPad Prism 8.0.

Significant tumor growth inhibition was observed for both the compounds at 225 mpk BID, as shown in Table 6 and Table 7 respectively. Both the compounds were well tolerated with <20% body weight change.

TABLE 6
Anti-tumor effect of Compound of Example 1 in
PANC-1 xenograft model in C.B-17 SCID mice

| Dose (mg/Kg) | Tumor Growth | |
|---|---|---|
| PO, | Inhibition % | |
| BID | (TGI %; day 28) | p-value |
| 150 | 49% | 0.0098 |
| 225 | 68% | 0.0010 |

TABLE 7
Anti-tumor effect of Compound of Example 2 in
PANC-1 xenograft model in C.B-17 SCID mice

| Dose (mg/Kg) | Tumor Growth | |
|---|---|---|
| PO, | Inhibition % | |
| BID | (TGI %; day 28) | p-value |
| 150 | 48% | 0.1326 |
| 225 | 70% | 0.0146 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I 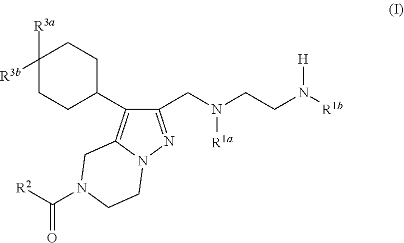
or a salt thereof, wherein:
R^{1a} and R^{1b} are independently chosen from H and CH₃;
R² is chosen from C₁₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.
R^{3a} and R^{3b} are independently chosen from (C₁₋₆alkoxy)C₁₋₆alkyl and (haloC₁₋₆alkoxy)C₁₋₆alkyl;
each R⁴ is independently chosen from halo, cyano, hydroxy, C₁₋₆haloalkyl, C₁₋₆alkoxy, and C₁₋₆haloalkoxy, and if R² is C₃₋₇cycloalkyl, 4- to 7-membered cycloalkoxy, or (C₃₋₇cycloalkyl)C₁₋₆alkyl, then R⁴ also may be C₁₋₆alkyl; and
provided the compound is not

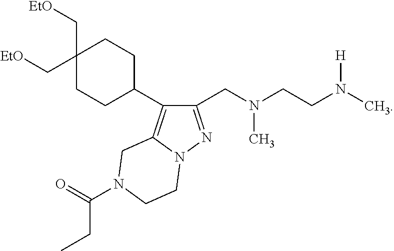 [SMILES: CCOCC1(COCC)CCC(c2c(CN(C)CCNC)nn3c2CN(C(=O)CC)CC3)CC1]

2. The compound as recited in claim 1, or a salt thereof, wherein R² is chosen from methyl, C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.

3. The compound as recited in either one of claims 1 and 2, or a salt thereof, wherein R^{3a} and R^{3b} are independently chosen from (C₁₋₆alkoxy)methyl and (haloC₁₋₆alkoxy)methyl.

4. The compound as recited in claim 3, or a salt thereof, wherein R^{3a} and R^{3b} are independently chosen from (methoxy)methyl and (ethoxy)methyl.

5. The compound as recited in claim 4, or a salt thereof, wherein R^{3a} and R^{3b} are (methoxy)methyl.

6. The compound as recited in any one of claims 1-5, or a salt thereof, wherein each R⁴ is independently chosen from halo, cyano, hydroxy, C₁₋₄fluoroalkyl, C₁₋₄alkoxy, and C₁₋₄fluoroalkoxy, and if R² is C₃₋₇cycloalkyl, 4- to 7-membered cycloalkoxy, or (C₃₋₇cycloalkyl)C₁₋₆alkyl, then R⁴ also may be C₁₋₄alkyl.

7. The compound as recited in any one of claims 1-6, or a salt thereof, wherein R² is chosen from C₃₋₆alkyl, C₃₋₇cycloalkyl, 4- to 7-membered cycloalkoxy, and (C₃₋₇cycloalkyl)C₁₋₆alkyl, any of which is optionally substituted with one or more R⁴.

8. The compound as recited in any one of claims 1-6, or a salt thereof, wherein R² is chosen from:

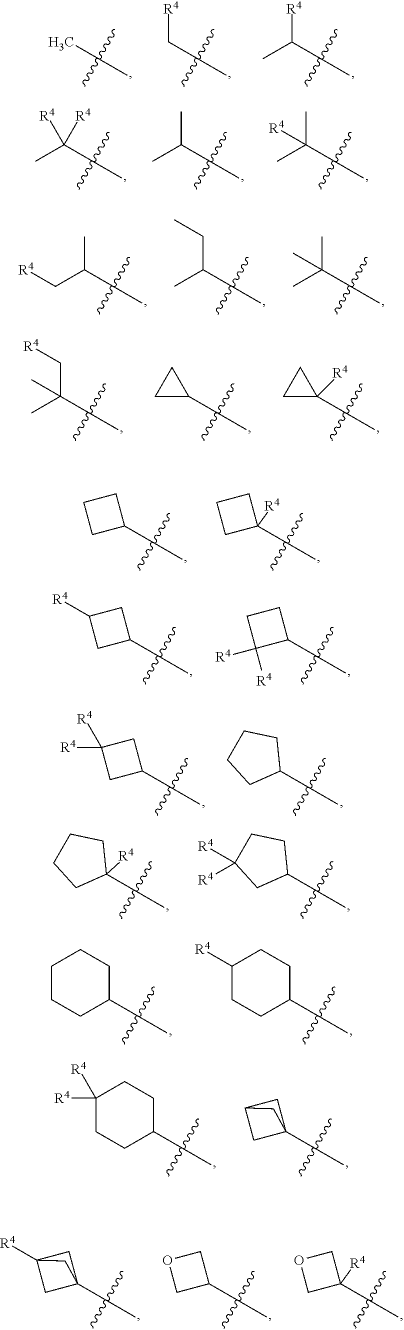

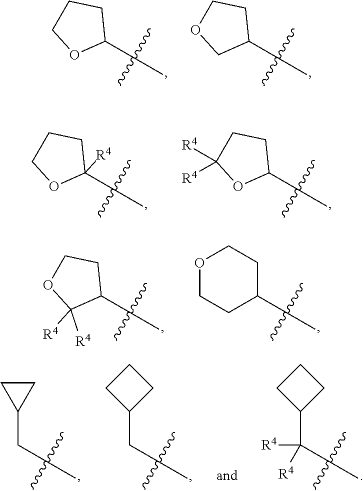

9. The compound as recited in any one of claims 1-6, or a salt thereof, wherein:
R² is chosen from 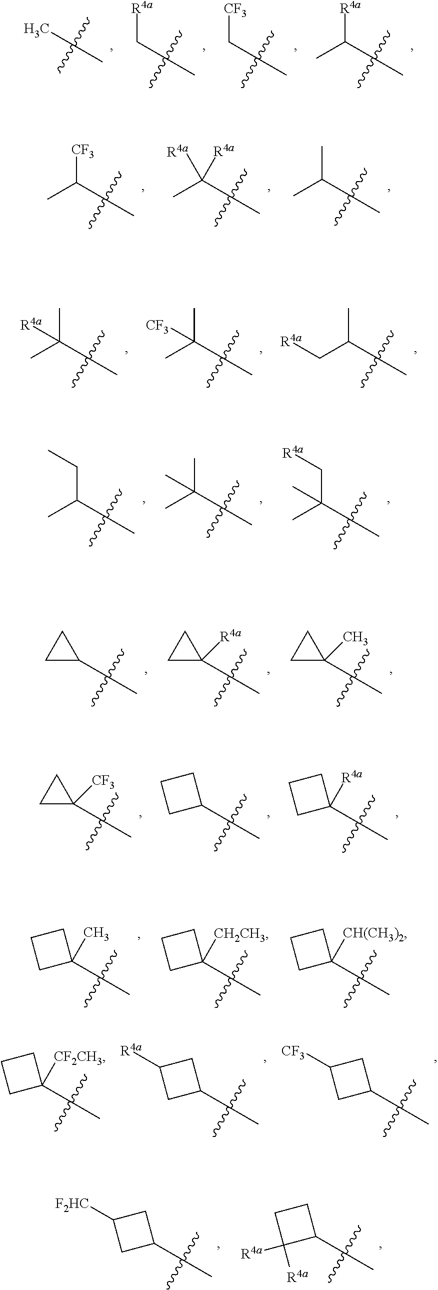 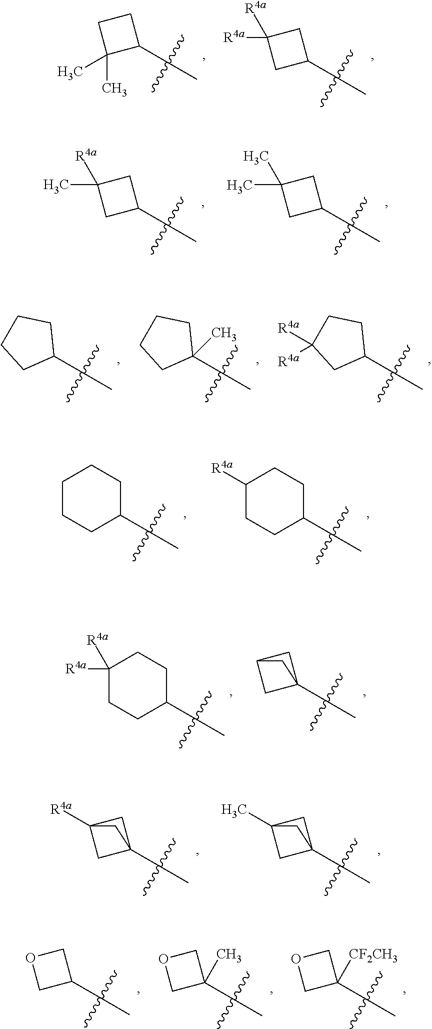 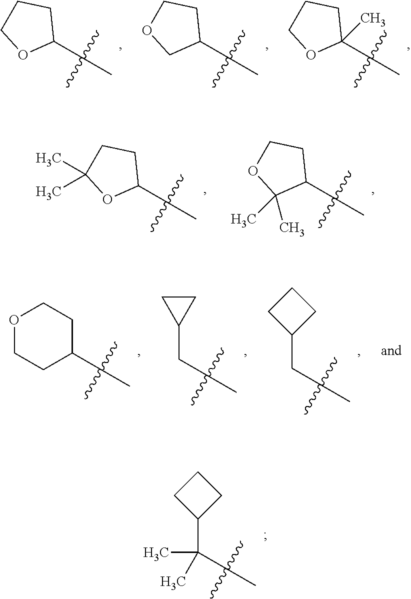
and
each R^{4a} is independently chosen from halo, cyano, hydroxy, C₁₋₄fluoroalkyl, C₁₋₄alkoxy, and C₁₋₄fluoroalkoxy.

10. The compound as recited in claim 9, or a salt thereof, wherein each R^{4a} is independently chosen from halo, cyano, hydroxy, C₁₋₄alkoxy, and C₁₋₄fluoroalkoxy.

11. The compound as recited in any one of claims 1-6, or a salt thereof, wherein:
R² is chosen from 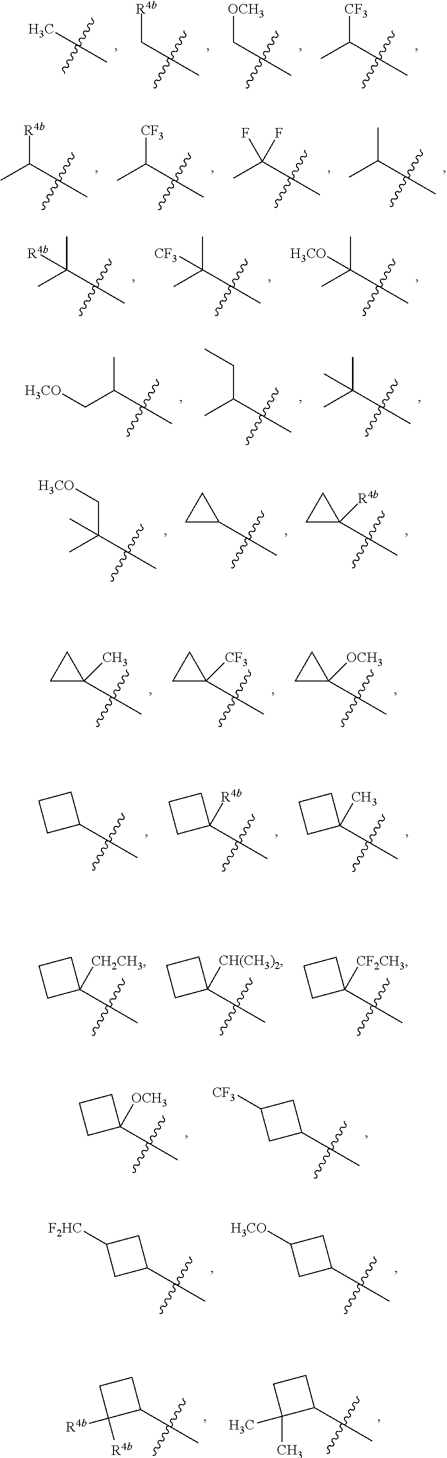 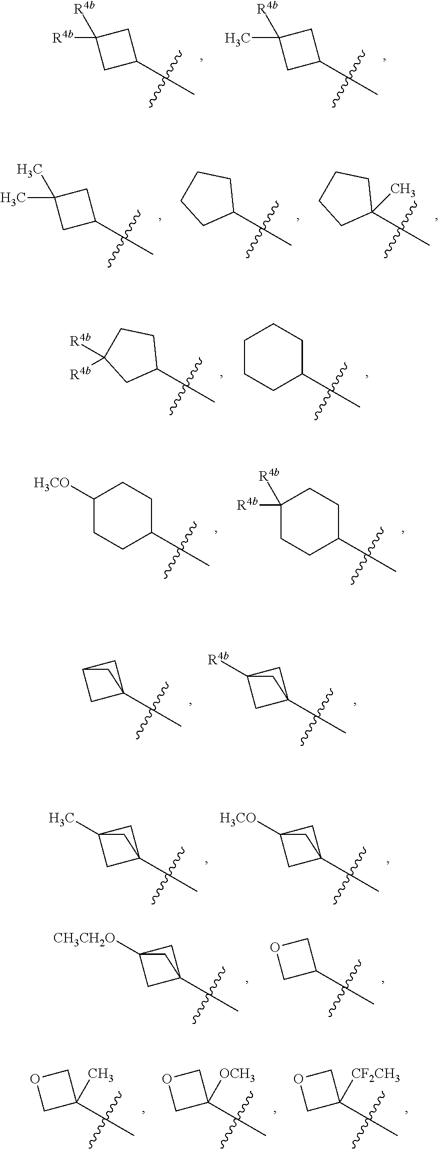 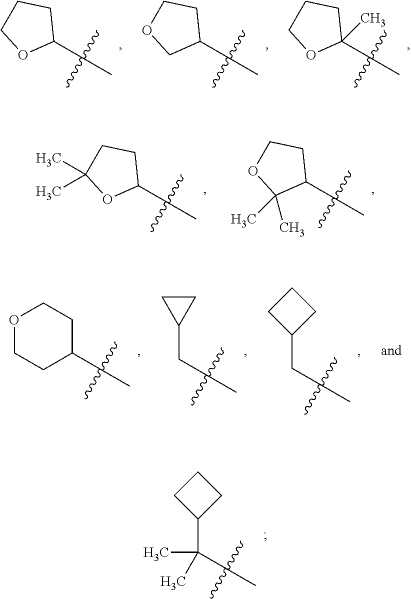
each R^{4b} is independently chosen from halo, cyano, hydroxy, C₁₋₄fluoroalkyl, C₁₋₄alkoxy, and C₁₋₄fluoroalkoxy.

12. The compound as recited in claim 11, or a salt thereof, wherein each R^{4b} is independently chosen from halo, cyano, and hydroxy.

13. The compound as recited in claim 12, or a salt thereof, wherein each R^{4b} is independently chosen from fluoro, chloro, cyano, and hydroxy.

14. The compound as recited in any one of claims 1-6, or a salt thereof, wherein:
R² is chosen from

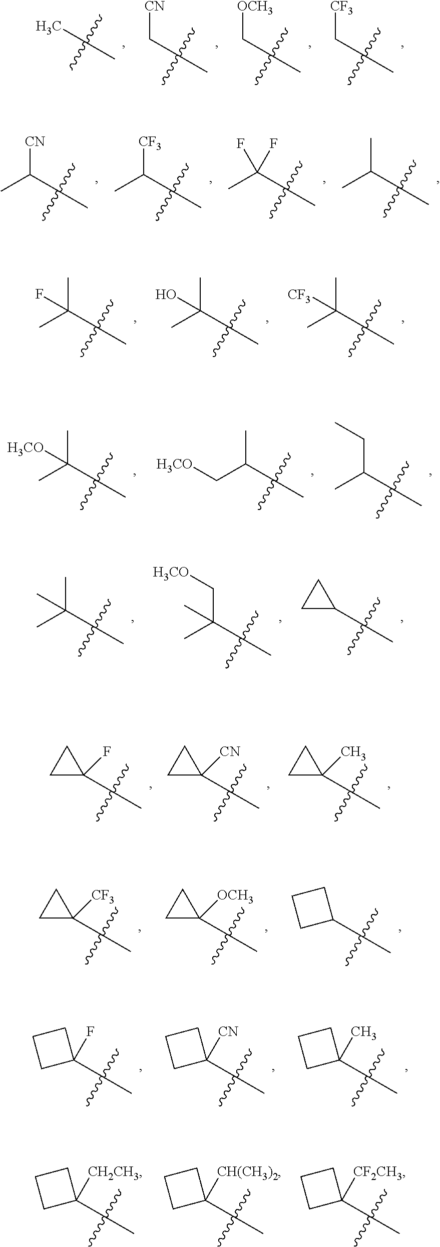

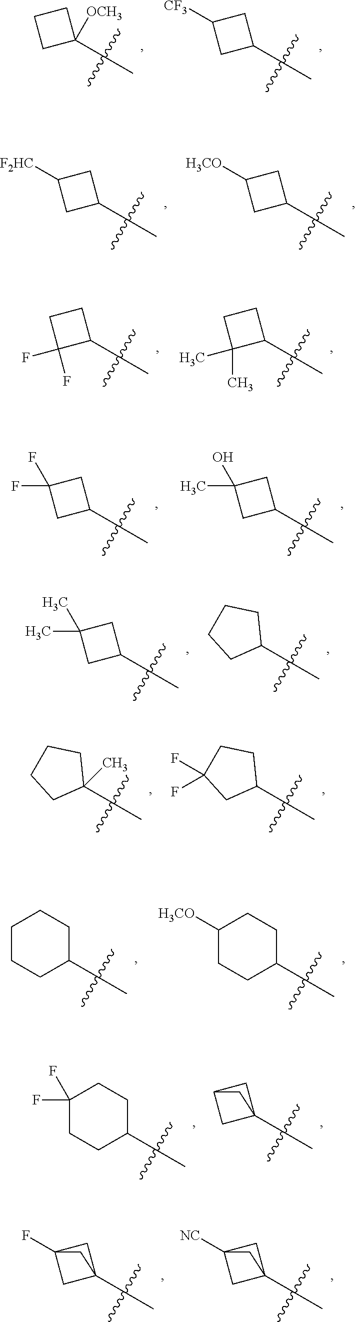

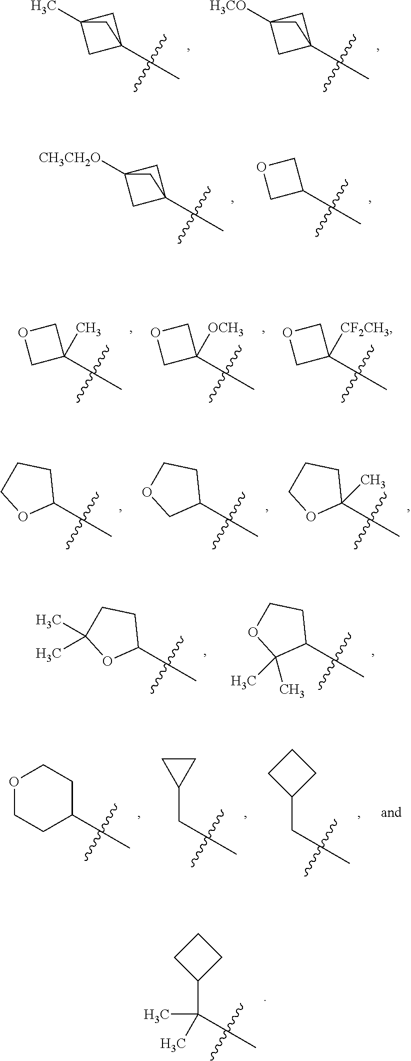

15. The compound as recited in any one of claims 1-6, or a salt thereof, wherein R is chosen from C₄alkyl, cyclobutyl, and oxetan-3-yl, any of which is optionally substituted with one or two R⁴.

16. The compound as recited in claim 15, or a salt thereof, wherein R² is chosen from C₄alkyl and cyclobutyl, either of which is optionally substituted with one or two R⁴.

17. The compound as recited in either one of claims 15 and 16, or a salt thereof, wherein each R⁴ is independently chosen from halo, cyano, and hydroxy.

18. The compound as recited in claim 17, or a salt thereof, wherein R⁴ is fluoro.

19. The compound as recited in any one of claims 1-6, or a salt thereof, wherein R² is chosen from cylopropyl, cyclobutyl, cyclopentyl, 2-propyl, 2-butyl, and 2-methyl-2-propyl.

20. The compound as recited in claim 19, or a salt thereof, wherein R² is chosen from cyclobutyl and 2-methyl-2-propyl.

21. The compound as recited in claim 1, chosen from: 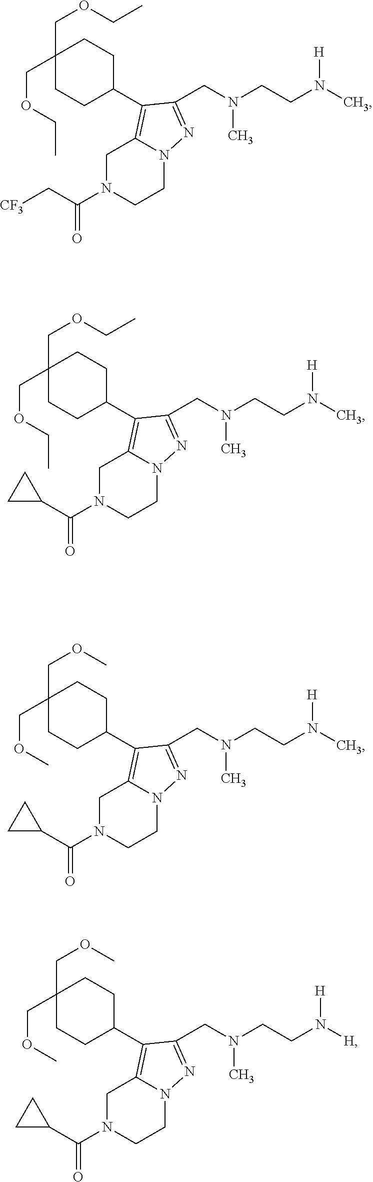 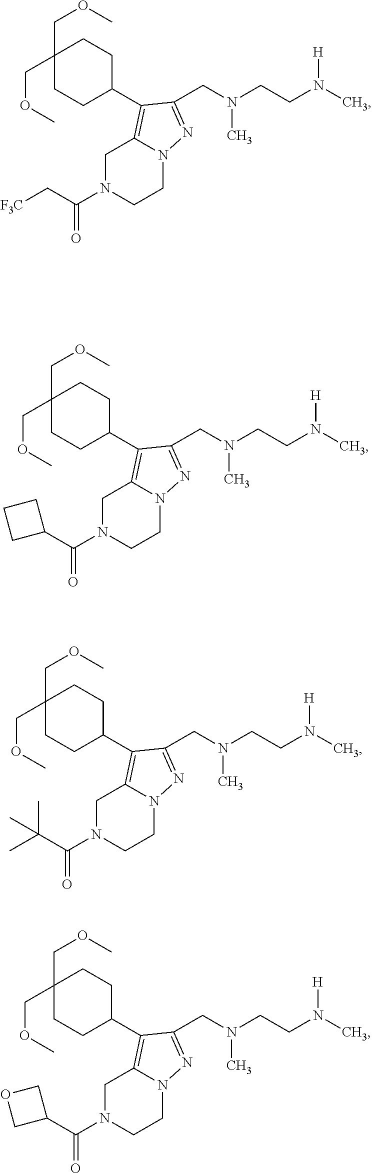 [SMILES: CNCCN(C)Cc1nn2c(c1C1CCC(COC)(COC)CC1)CN(C(=O)CC(F)(F)F)CC2 ; CNCCN(C)Cc1nn2c(c1C1CCC(COC)(COC)CC1)CN(C(=O)C1CCC1)CC2 ; CNCCN(C)Cc1nn2c(c1C1CCC(COC)(COC)CC1)CN(C(=O)C(C)(C)C)CC2 ; CNCCN(C)Cc1nn2c(c1C1CCC(COC)(COC)CC1)CN(C(=O)C1COC1)CC2] 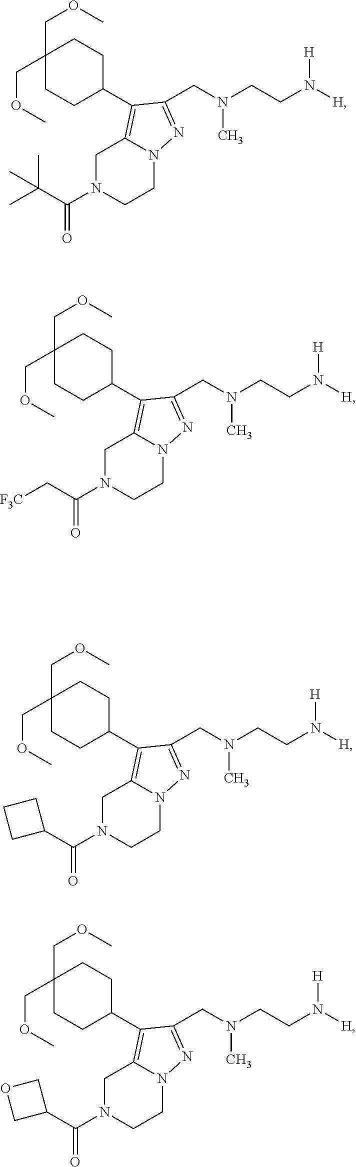 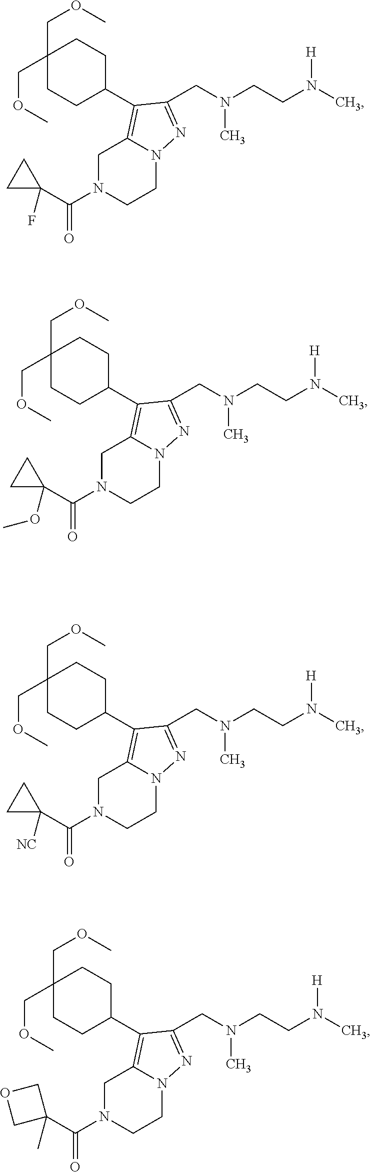 [SMILES: CNCCN(C)Cc1nn2c(c1C1CCC(COC)(COC)CC1)CN(C(=O)C1(F)CC1)CC2 ; CNCCN(C)Cc1nn2c(c1C1CCC(COC)(COC)CC1)CN(C(=O)C1(OC)CC1)CC2 ; CNCCN(C)Cc1nn2c(c1C1CCC(COC)(COC)CC1)CN(C(=O)C1(C#N)CC1)CC2 ; CNCCN(C)Cc1nn2c(c1C1CCC(COC)(COC)CC1)CN(C(=O)C1(C)COC1)CC2] 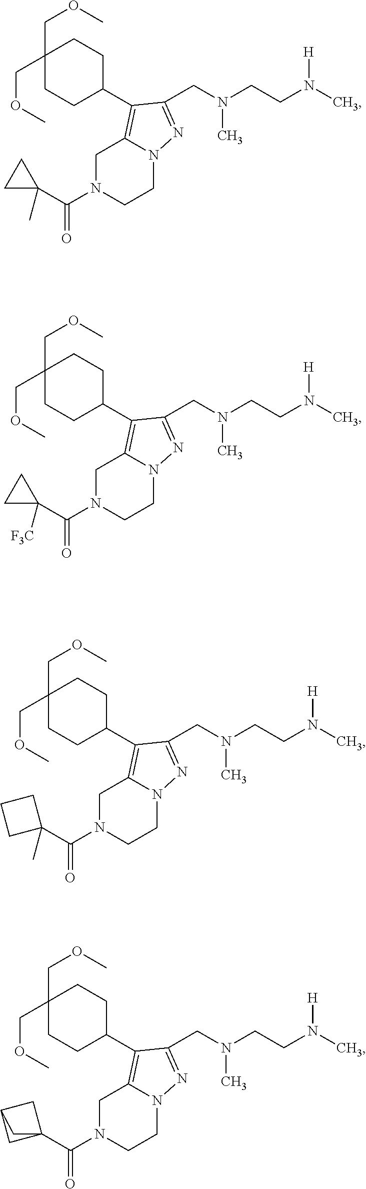 [SMILES: CNCCN(C)Cc1nn2c(c1C1CCC(COC)(COC)CC1)CN(C(=O)C1(C)CC1)CC2 ; CNCCN(C)Cc1nn2c(c1C1CCC(COC)(COC)CC1)CN(C(=O)C1(C(F)(F)F)CC1)CC2 ; CNCCN(C)Cc1nn2c(c1C1CCC(COC)(COC)CC1)CN(C(=O)C1(C)CCC1)CC2 ; CNCCN(C)Cc1nn2c(c1C1CCC(COC)(COC)CC1)CN(C(=O)C13CC(C1)C3)CC2] 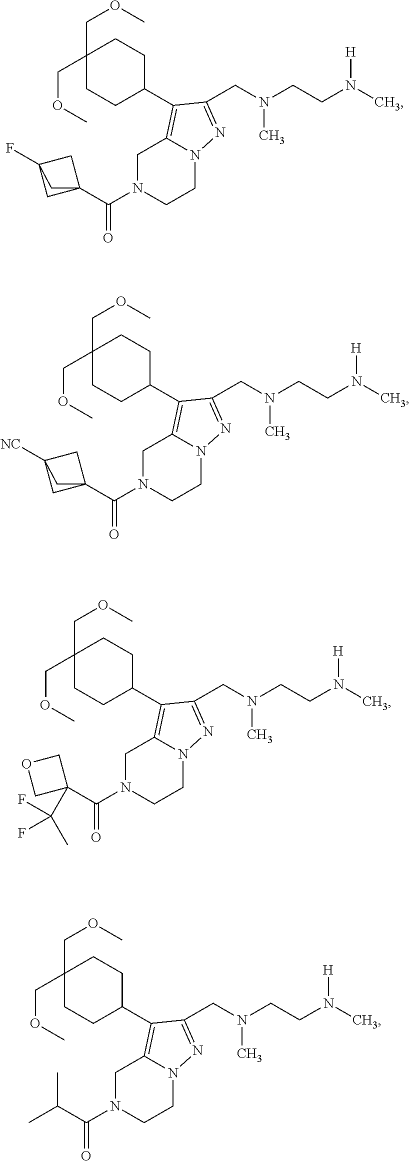 [SMILES: CNCCN(C)Cc1nn2c(c1C1CCC(COC)(COC)CC1)CN(C(=O)C13CC(F)(C1)C3)CC2 ; CNCCN(C)Cc1nn2c(c1C1CCC(COC)(COC)CC1)CN(C(=O)C13CC(C#N)(C1)C3)CC2 ; CNCCN(C)Cc1nn2c(c1C1CCC(COC)(COC)CC1)CN(C(=O)C1(C(C)(F)F)COC1)CC2 ; CNCCN(C)Cc1nn2c(c1C1CCC(COC)(COC)CC1)CN(C(=O)C(C)C)CC2] 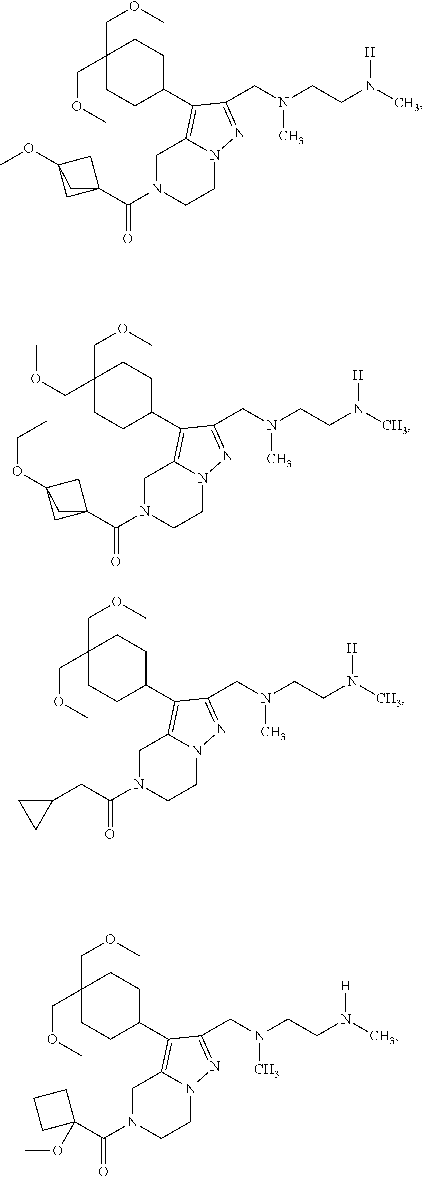 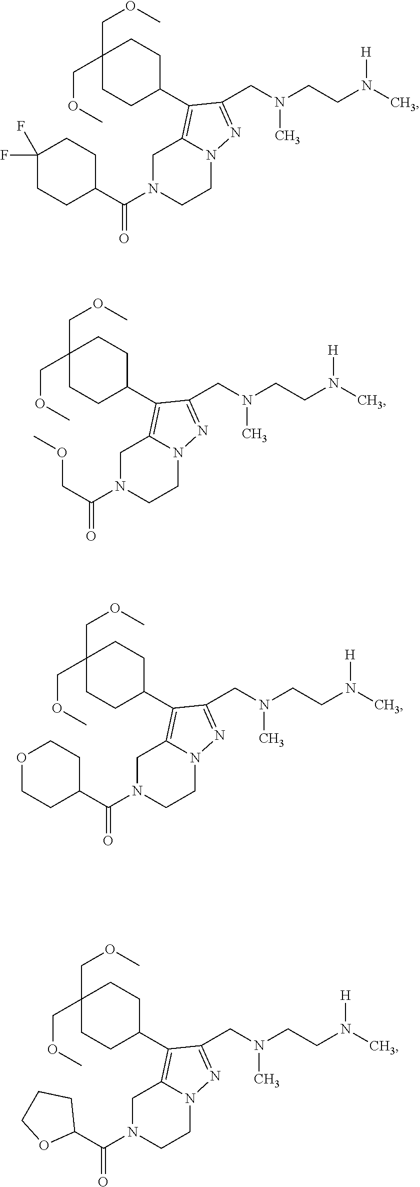 [SMILES: CNCCN(C)Cc1nn2c(c1C1CCC(COC)(COC)CC1)CN(C(=O)C1CCC(F)(F)CC1)CC2 ; CNCCN(C)Cc1nn2c(c1C1CCC(COC)(COC)CC1)CN(C(=O)COC)CC2 ; CNCCN(C)Cc1nn2c(c1C1CCC(COC)(COC)CC1)CN(C(=O)C1CCOCC1)CC2 ; CNCCN(C)Cc1nn2c(c1C1CCC(COC)(COC)CC1)CN(C(=O)C1CCCO1)CC2] 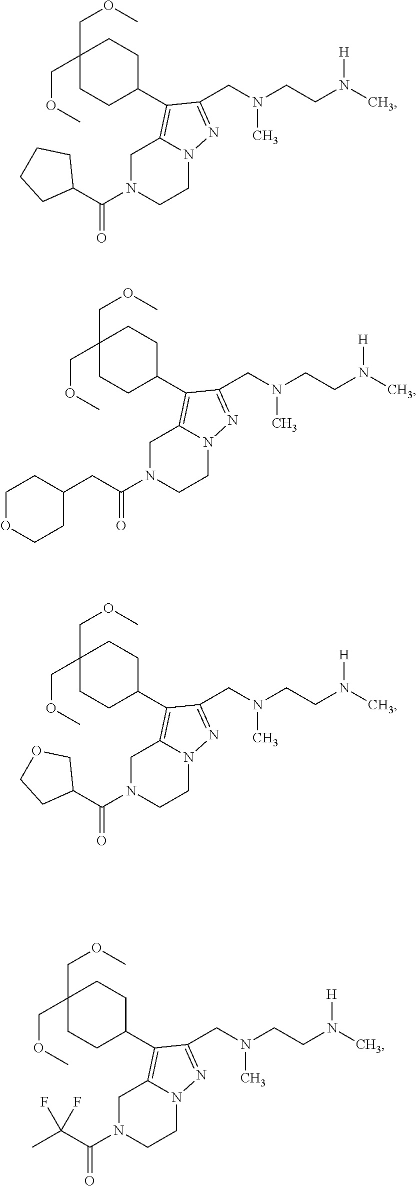 [SMILES: CNCCN(C)Cc1nn2c(c1C1CCC(COC)(COC)CC1)CN(C(=O)C1CCCC1)CC2 ; CNCCN(C)Cc1nn2c(c1C1CCC(COC)(COC)CC1)CN(C(=O)CC1CCOCC1)CC2 ; CNCCN(C)Cc1nn2c(c1C1CCC(COC)(COC)CC1)CN(C(=O)C1CCOC1)CC2 ; CNCCN(C)Cc1nn2c(c1C1CCC(COC)(COC)CC1)CN(C(=O)C(C)(F)F)CC2] 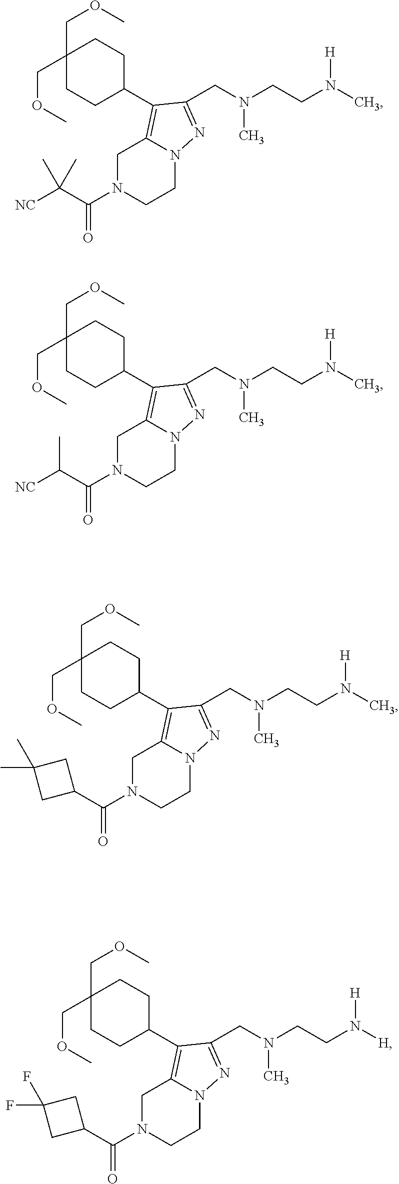 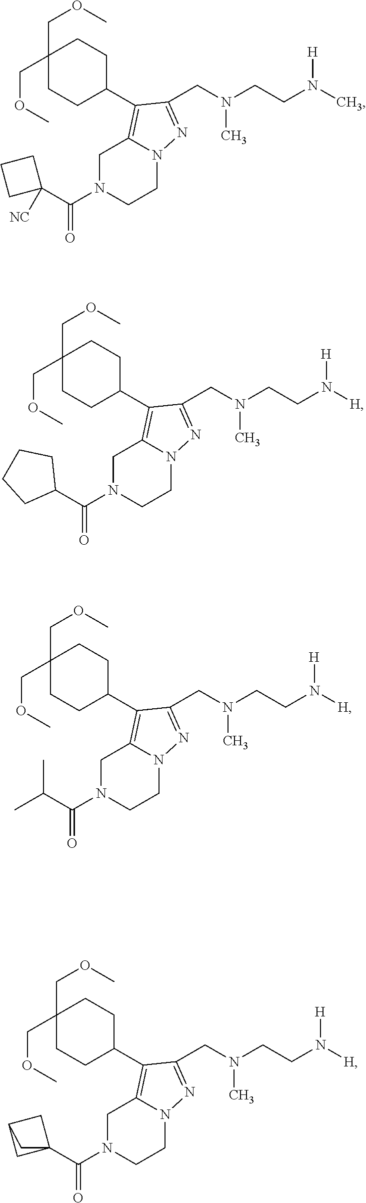 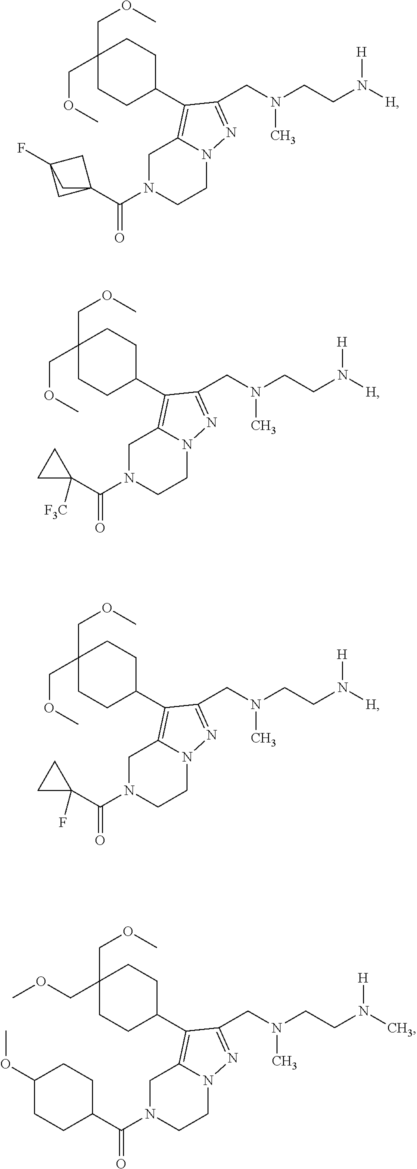 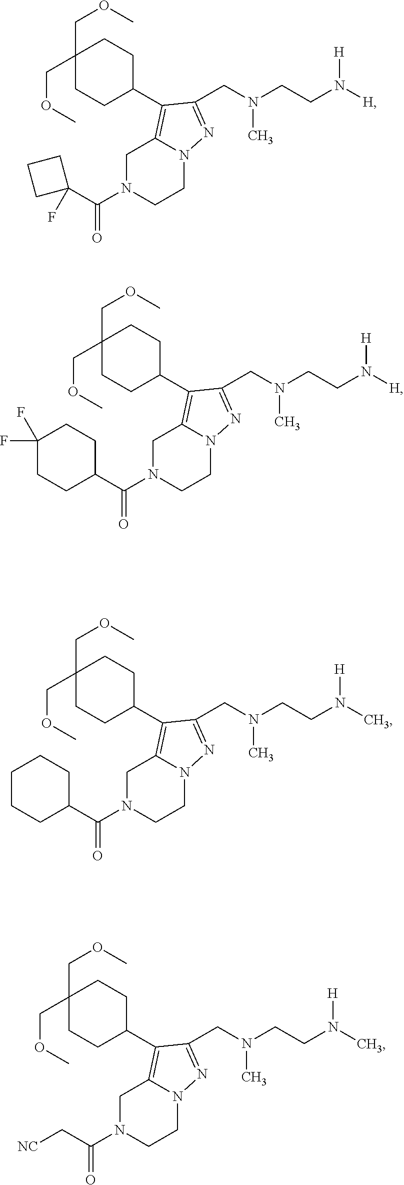 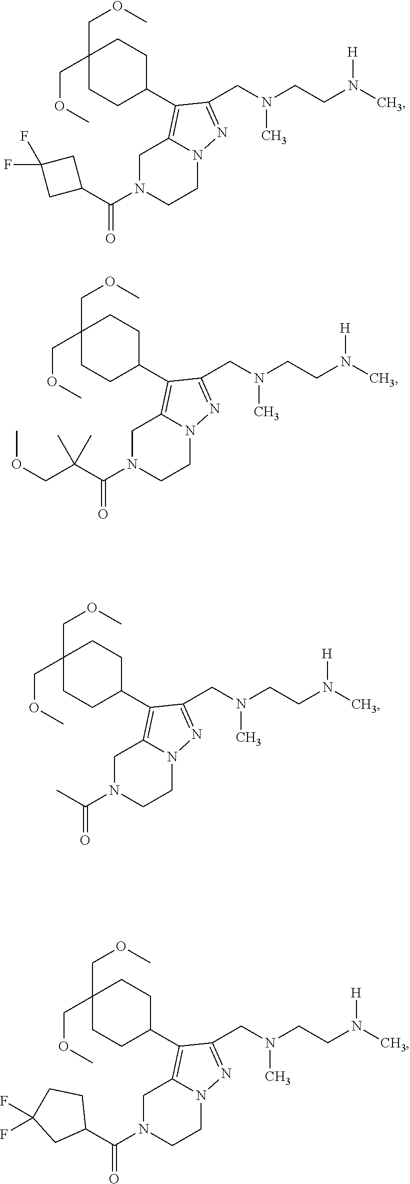 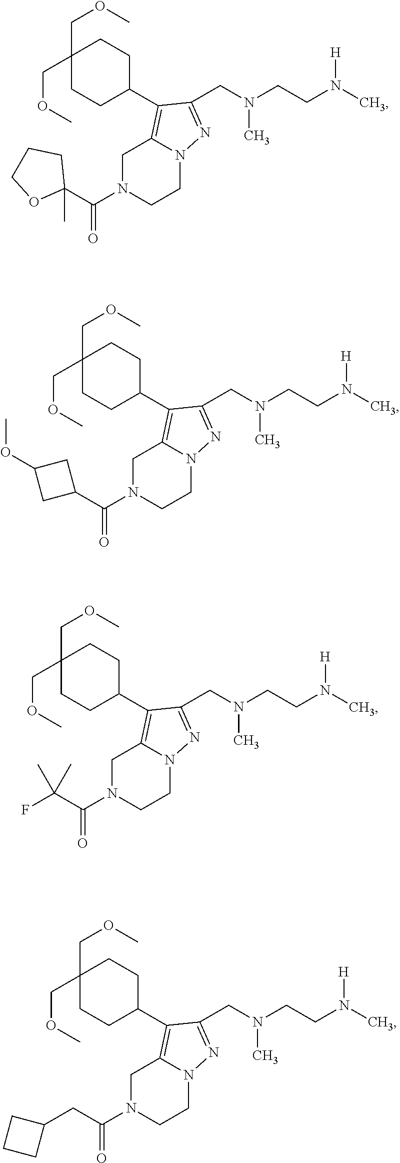 [SMILES: CNCCN(C)Cc1nn2c(c1C1CCC(COC)(COC)CC1)CN(C(=O)C1(C)CCCO1)CC2 ; CNCCN(C)Cc1nn2c(c1C1CCC(COC)(COC)CC1)CN(C(=O)C1CC(OC)C1)CC2 ; CNCCN(C)Cc1nn2c(c1C1CCC(COC)(COC)CC1)CN(C(=O)C(C)(C)F)CC2 ; CNCCN(C)Cc1nn2c(c1C1CCC(COC)(COC)CC1)CN(C(=O)CC1CCC1)CC2] 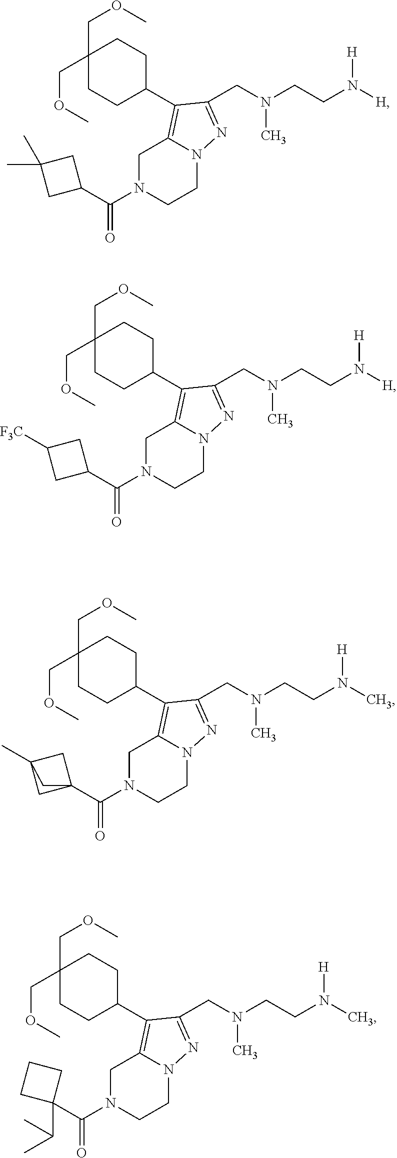 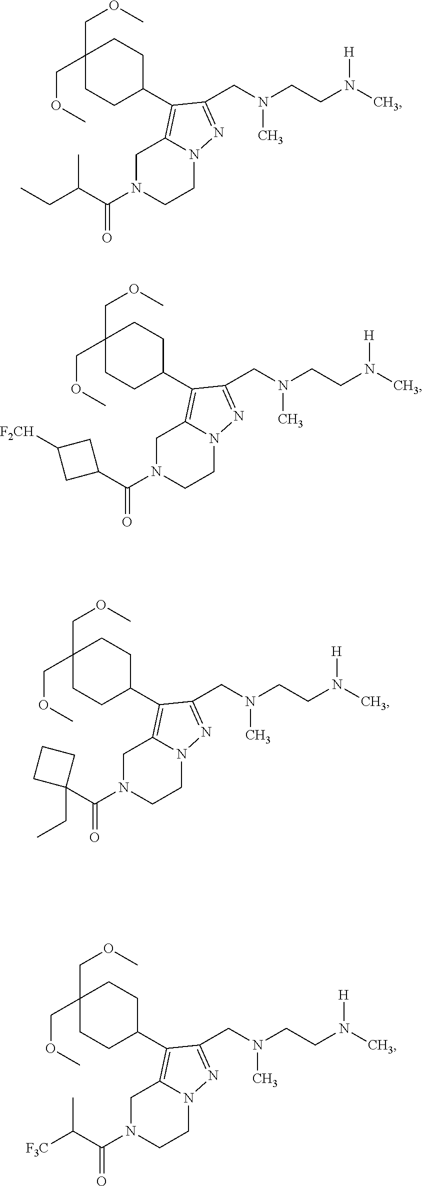 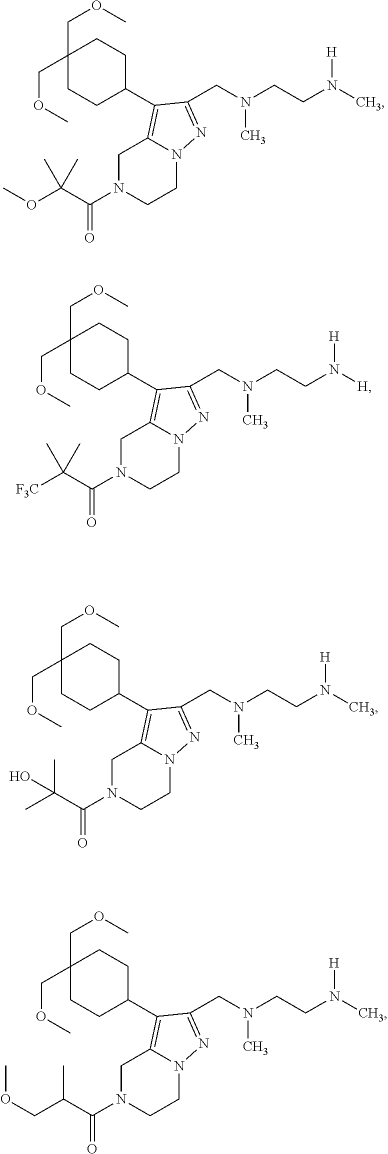 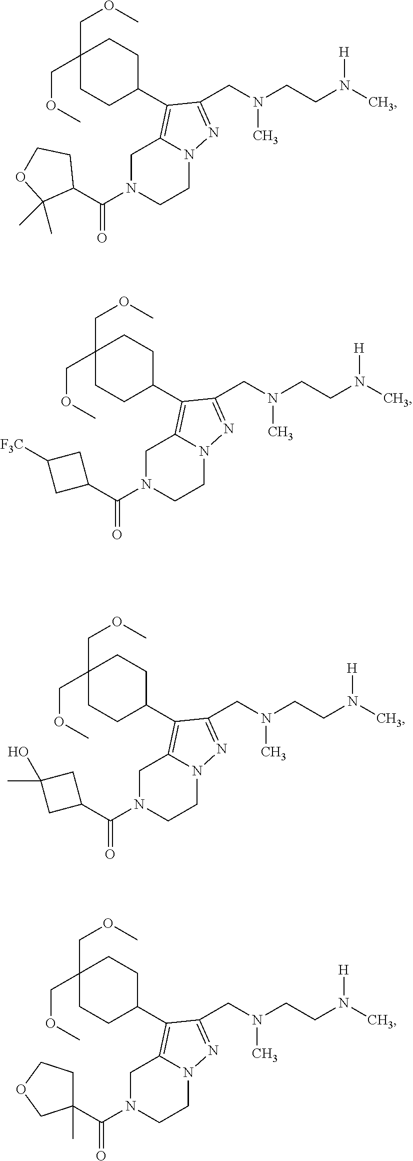 [SMILES: CNCCN(C)Cc1nn2c(c1C1CCC(COC)(COC)CC1)CN(C(=O)C1CCOC1(C)C)CC2 ; CNCCN(C)Cc1nn2c(c1C1CCC(COC)(COC)CC1)CN(C(=O)C1CC(C(F)(F)F)C1)CC2 ; CNCCN(C)Cc1nn2c(c1C1CCC(COC)(COC)CC1)CN(C(=O)C1CC(C)(O)C1)CC2 ; CNCCN(C)Cc1nn2c(c1C1CCC(COC)(COC)CC1)CN(C(=O)C1(C)CCOC1)CC2] 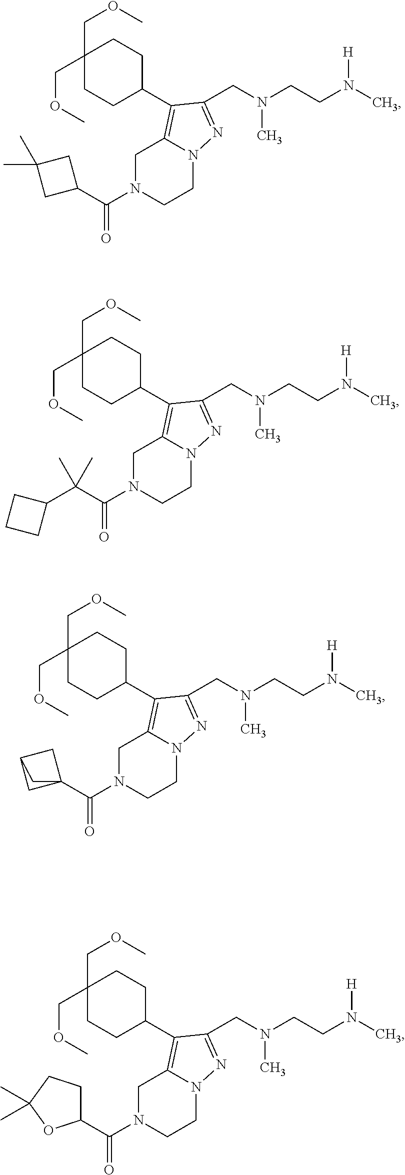 [SMILES: CNCCN(C)Cc1nn2c(c1C1CCC(COC)(COC)CC1)CN(C(=O)C1CC(C)(C)C1)CC2 ; CNCCN(C)Cc1nn2c(c1C1CCC(COC)(COC)CC1)CN(C(=O)C(C)(C)C1CCC1)CC2 ; CNCCN(C)Cc1nn2c(c1C1CCC(COC)(COC)CC1)CN(C(=O)C13CC(C1)C3)CC2 ; CNCCN(C)Cc1nn2c(c1C1CCC(COC)(COC)CC1)CN(C(=O)C1CCC(C)(C)O1)CC2] 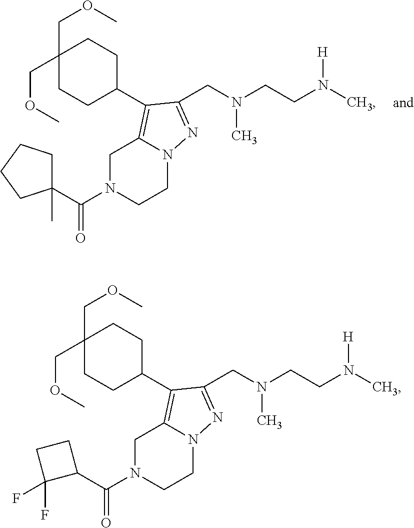 [SMILES: CNCCN(C)Cc1nn2c(c1C1CCC(COC)(COC)CC1)CN(C(=O)C1(C)CCCC1)CC2 ; CNCCN(C)Cc1nn2c(c1C1CCC(COC)(COC)CC1)CN(C(=O)C1CCC1(F)F)CC2]
or a salt thereof.

22. A compound as recited in any one of claims 1-21, or a salt thereof, for use as a medicament.

23. A compound as recited in any one of claims 1-21, or a salt thereof, for use in the treatment of cancer.

24. A compound as recited in any one of claims 1-21, or a salt thereof, for use in the manufacture of a medicament for the prevention or treatment of a disease or condition ameliorated by the inhibition of PRMT.

25. A pharmaceutical composition comprising a compound as recited in any one of claims 1-21, or a salt thereof, together with a pharmaceutically acceptable carrier.

26. A method of inhibition of a PRMT comprising contacting PRMT with a compound as recited in any one of claims 1-21, or a salt thereof.

27. The method as recited in claim 26, wherein the PRMT is PRMT1.

28. A method of modulating gene expression comprising contacting a cell with an effective dose of the compound as recited in any one of claims 1-21, or a salt thereof.

29. A method of treatment of a PRMT-mediated disease comprising the administration of a therapeutically effective amount of a compound as recited in any one of claims 1-21, or a salt thereof, to a patient in need thereof.

30. The method of claim 29, wherein the disease is a proliferative disease.

31. The method of claim 30, wherein the proliferative disease is cancer.

32. The method of claim 31, wherein the cancer is chosen from acoustic neuroma, acute leukemia, acute lymphocytic leukemia, acute myelocytic leukemia, acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

33. The method as recited in claim 31, further comprising the administration of a non-chemical method of cancer treatment.

34. The method as recited in claim 33, wherein said non-chemical method of cancer treatment is chosen from surgery, radiation therapy, thermoablation, focused ultrasound therapy, and cryotherapy.

35. A method of treatment of a PRMT-mediated disease comprising the administration of:
a. a therapeutically effective amount of a compound as recited in any one of claims 1-21, or a salt thereof; and
b. another therapeutic agent.

36. The method as recited in claim 35, wherein said other agent is a cytotoxic agent.

37. The method as recited in claim 36, wherein said cytotoxic agent is chosen from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism.

38. The method as recited in claim 29, wherein the disease is an autoimmune disease.

39. The method as recited in claim 29, wherein the disease is amyotrophic lateral sclerosis.

40. The method as recited in claim 29, wherein the disease is a muscular dystrophy.

41. The method as recited in claim 29, wherein the disease is a vascular disease.

42. The method as recited in claim 29, wherein the disease is a metabolic disorder.

43. The method as recited in claim 42, wherein the metabolic disorder is diabetes.

44. The method as recited in claim 42, wherein the metabolic disorder is a skeletal muscle metabolic disorder.
